(19) United States
(12) Patent Application Publication
(10) Pub. No.: US 2017/0121322 A1
(43) Pub. Date: May 4, 2017

(54) BET-PROTEIN INHIBITING 3,4-DIHYDROPYRIDO[2,3-B]PYRAZINONES WITH META-SUBSTITUTED AROMATIC AMINO- OR ETHER GROUPS

(71) Applicant: Bayer Pharma Aktiengesellschaft, Berlin (DE)

(72) Inventors: Norbert SCHMEES, Berlin (DE); Bernard HAENDLER, Berlin (DE); Detlef STÖCKIGT, Potsdam (DE); Richard Alexander BISSELL, Marple Bridge, Stockport (GB); Richard Alexander BOUGLAS, Buxton (GB); Ian STEFANUTI, Chapel-en-le-Frith, High Peak (GB); Stephan SIEGEL, Berlin (DE)

(73) Assignee: Bayer Pharma Aktiengesellschaft, Berlin (DE)

(21) Appl. No.: 15/317,925

(22) PCT Filed: Jun. 15, 2015
(86) PCT No.: PCT/EP2015/063278

§ 371 Date: Dec. 9, 2016

(30) Foreign Application Priority Data
Jun. 18, 2014 (EP) 14173045.7

(51) Int. Cl. C07D 471/04

(52) U.S. Cl. CPC C07D 471/04

(57) ABSTRACT

The present invention relates to BET protein-inhibitory, especially BRD4-inhibitory 3,4-dihydropyrido[2,3-b]pyrazinones with a meta-substituted aromatic amino or ether group of the general formula (I)

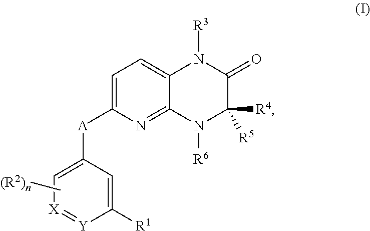

in which A, X, Y, R¹, R², R³, R⁴, R⁵, R⁶ and n are each as defined in the description, to pharmaceutical compositions comprising the compounds according to the invention, and to the prophylactic and therapeutic use thereof in the case of hyperproliferative disorders, especially in the case of tumour disorders.

Furthermore, this invention relates to the use of BET protein inhibitors in viral infections, in neurodegenerative disorders, in inflammatory diseases, in atherosclerotic disorders and in male fertility control.

The present invention relates to BET protein-inhibitory, especially BRD4-inhibitory 3,4-dihydropyrido[2,3-b]pyrazinones with a meta-substituted aromatic amino or ether group, to pharmaceutical compositions comprising the inventive compounds, and to the prophylactic and therapeutic use thereof in the case of hyperproliferative disorders, especially in the case of neoplastic disorders. Furthermore, this invention relates to the use of BET protein inhibitors in viral infections, in neurodegenerative disorders, in inflammatory diseases, in atherosclerotic disorders and in male fertility control.

The human BET family (bromodomain and extra C-terminal domain family) has four members (BRD2, BRD3, BRD4 and BRDT) containing two related bromodomains and one extraterminal domain (Wu and Chiang, J. Biol. Chem., 2007, 282:13141-13145). The bromodomains are protein regions which recognize acetylated lysine residues. Such acetylated lysines are often found at the N-terminal end of histones (e.g. histone H3 or histone H4) and are features of an open chromatin structure and active gene transcription (Kuo and Allis, Bioessays, 1998, 20:615-626). In addition, bromodomains can recognize further acetylated proteins. For example, BRD4 binds to RelA, which leads to stimulation of NF-κB and transcriptional activity of inflammatory genes (Huang et al., Mol. Cell. Biol., 2009, 29:1375-1387). BRD4 also binds to cyclin T1 and forms an active complex which is important for transcription elongation (Schrider et al., J. Biol. Chem., 2012, 287:1090-1099). The extraterminal domain of BRD2, BRD3 and BRD4 interacts with several proteins involved in chromatin modulation and the regulation of gene expression (Rahman et al., Mol. Cell. Biol., 2011, 31:2641-2652).

In mechanistic terms, BET proteins play an important role in cell growth and in the cell cycle. They are associated with mitotic chromosomes, which suggests a role in epigenetic memory (Dey et al., Mol. Biol. Cell, 2009, 20:4899-4909; Yang et al., Mol. Cell. Biol., 2008, 28:967-976). Involvement of BRD4 in the post-mitotic reactivation of gene transcription has been demonstrated (Zhao et al., Nat. Cell. Biol., 2011, 13:1295-1304). BRD4 is essential for transcription elongation and recruits the elongation complex P-TEFb consisting of CDK9 and cyclin T1, which leads to activation of RNA polymerase II (Yang et al., Mol. Cell, 2005, 19:535-545; Schröder et al., J. Biol. Chem., 2012, 287:1090-1099). Consequently, the expression of genes involved in cell proliferation is stimulated, for example of c-Myc, cyclin D1 and aurora B (You et al., Mol. Cell. Biol., 2009, 29:5094-5103; Zuber et al., Nature, 2011, doi: 10.1038). BRD2 is involved in the regulation of target genes of the androgen receptor (Draker et al., PLOS Genetics, 2012, 8, e1003047). BRD2 and BRD3 bind to transcribed genes in hyperacetylated chromatin regions and promote transcription by RNA polymerase II (LeRoy et al., Mol. Cell, 2008, 30:51-60).

The knockdown of BRD4 or the inhibition of the interaction with acetylated histones in various cell lines leads to a G1 arrest (Mochizuki et al., J. Biol. Chem., 2008, 283:9040-9048; Mertz et al., Proc. Natl. Acad. Sci. USA, 2011, 108:16669-16674). It has also been shown that BRD4 binds to promoter regions of several genes which are activated in the G1 phase, for example cyclin D1 and D2 (Mochizuki et al., J. Biol. Chem., 2008, 283:9040-9048). In addition, inhibition of the expression of c-Myc, an essential factor in cell proliferation, after BRD4 inhibition has been demonstrated (Dawson et al., Nature, 2011, 478:529-533; Delmore et al., Cell, 2011, 146:1-14; Mertz et al., Proc. Natl. Acad. Sci. USA, 2011, 108:16669-16674). Inhibition of the expression of androgen-regulated genes and binding of BRD2 to corresponding regulatory regions has also been demonstrated (Draker et al., PLOS Genetics, 2012, 8, e1003047).

BRD2 and BRD4 knockout mice die early in embryogenesis (Gyuris et al., Biochim. Biophys. Acta, 2009, 1789:413-421; Houzelstein et al., Mol. Cell. Biol., 2002, 22:3794-3802). Heterozygotic BRD4 mice have various growth defects attributable to reduced cell proliferation (Houzelstein et al., Mol. Cell. Biol., 2002, 22:3794-3802).

BET proteins play an important role in various tumour types. Fusion between the BET proteins BRD3 or BRD4 and NUT, a protein which is normally expressed only in the testes, leads to an aggressive form of squamous cell carcinoma, called NUT midline carcinoma (French, Cancer Genet. Cytogenet., 2010, 203:16-20). The fusion protein prevents cell differentiation and promotes proliferation (Yan et al., J. Biol. Chem., 2011, 286:27663-27675). The growth of in vivo models derived therefrom is inhibited by a BRD4 inhibitor (Filippakopoulos et al., Nature, 2010, 468:1067-1073). Screening for therapeutic targets in an acute myeloid leukaemia cell line (AML) showed that BRD4 plays an important role in this tumour (Zuber et al., Nature, 2011, 478, 524-528). Reduction in BRD4 expression leads to a selective arrest of the cell cycle and to apoptosis. Treatment with a BRD4 inhibitor prevents the proliferation of an AML xenograft in vivo. Further experiments with a BRD4 inhibitor show that BRD4 is involved in various haematological tumours, for example multiple myeloma (Delmore et al., Cell, 2011, 146, 904-917) and Burkitt's lymphoma (Mertz et al., Proc. Natl. Acad. Sci. USA, 2011, 108, 16669-16674). In solid tumours too, for example lung cancer, BRD4 plays an important role (Lockwood et al., Proc. Natl. Acad. Sci. USA, 2012, 109, 19408-19413). Elevated expression of BRD4 has been detected in multiple myeloma, and amplification of the BRD4 gene has also been found in patients having multiple myeloma (Delmore et al., Cell, 2011, 146, 904-917). Amplification of the DNA region containing the BRD4 gene was detected in primary breast tumours (Kadota et al., Cancer Res, 2009, 69:7357-7365). For BRD2 too, there are data relating to a role in tumours. A transgenic mouse which overexpresses BRD2 selectively in B cells develops B cell lymphomas and leukaemias (Greenwall et al., Blood, 2005, 103:1475-1484).

BET proteins are also involved in viral infections. BRD4 binds to the E2 protein of various papillomaviruses and is important for the survival of the viruses in latently infected cells (Wu et al., Genes Dev., 2006, 20:2383-2396; Vosa et al., J. Virol., 2006, 80:8909-8919). The herpes virus, which is responsible for Kaposi's sarcoma, also interacts with various BET proteins, which is important for disease survival (Viejo-Borbolla et al., J. Virol., 2005, 79:13618-13629; You et al., J. Virol., 2006, 80:8909-8919). Through binding to P-TEFb, BRD4 also plays an important role in the replication of HIV-1 (Bisgrove et al., Proc. Natl Acad. Sci. USA, 2007, 104:13690-13695). Treatment with a BRD4 inhibitor leads to stimulation of the dormant, untreatable reservoir of HIV-1 viruses in T cells (Banerjee et al., J. Leukoc. Biol., 2012, 92, 1147-1154). This reactivation could enable new therapeutic methods for AIDS treatment (Zinchenko et al., J. Leukoc. Biol., 2012, 92, 1127-1129). A critical role of BRD4 in DNA replication of polyomaviruses has also been reported (Wang et al., PLoS Pathog., 2012, 8, doi:10.1371).

BET proteins are additionally involved in inflammation processes. BRD2-hypomorphic mice show reduced inflammation in adipose tissue (Wang et al., Biochem. J., 2009, 425:71-83). Infiltration of macrophages in white adipose tissue is also reduced in BRD2-deficient mice (Wang et al., Biochem. J., 2009, 425:71-83). It has also been shown that BRD4 regulates a number of genes involved in inflammation. In LPS-stimulated macrophages, a BRD4 inhibitor prevents the expression of inflammatory genes, for example IL-1 or IL-6 (Nicodeme et al., Nature, 2010, 468:1119-1123).

BET proteins are also involved in the regulation of the ApoA1 gene (Mirguet et al., Bioorg. Med. Chem. Lett., 2012, 22:2963-2967). The corresponding protein is part of high-density lipoprotein (HDL), which plays an important role in atherosclerosis (Smith, Arterioscler. Thromb. Vasc. Biol., 2010, 30:151-155). Through the stimulation of ApoA1 expression, BET protein inhibitors can increase the concentrations of cholesterol HDL and hence may potentially be useful for the treatment of atherosclerosis (Mirguet et al., Bioorg. Med. Chem. Lett., 2012, 22:2963-2967).

The BET protein BRDT plays an essential role in spermatogenesis through the regulation of the expression of several genes important during and after meiosis (Shang et al., Development, 2007, 134:3507-3515; Matzuk et al., Cell, 2012, 150:673-684). In addition, BRDT is involved in the post-meiotic organization of chromatin (Dhar et al., J. Biol. Chem., 2012, 287:6387-6405). In vivo experiments in mice show that treatment with a BET inhibitor which also inhibits BRDT leads to a decrease in sperm production and infertility (Matzuk et al., Cell, 2012, 150:673-684).

All these studies show that the BET proteins play an essential role in various pathologies, and also in male fertility. It would therefore be desirable to find potent and selective inhibitors which prevent the interaction between the BET proteins and acetylated proteins, in particular acetylated histone H4 peptides. These novel inhibitors should also have suitable pharmacokinetic properties which allow inhibition of these interactions in vivo, i.e. in patients.

It has now been found that 3,4-dihydropyrido[2,3-b]pyrazinones with a meta-substituted aromatic amino or ether group have the desired properties, i.e. they exhibit a BET protein-inhibitory, especially a BRD4 protein-inhibitory, effect. The compounds according to the invention are thus valuable active compounds for prophylactic and therapeutic use in the case of hyperproliferative disorders, especially in the case of neoplastic disorders. In addition, the compounds according to the invention can be used in the case of viral infections, in the case of neurodegenerative disorders, in the case of inflammatory diseases, in the case of atherosclerotic disorders and in male fertility control.

### PRIOR ART

The nomenclature applied in the assessment of the prior art (derived from the nomenclature software ACD Name batch, Version 12.01, from Advanced Chemical Development, Inc.) is illustrated by the following figures:

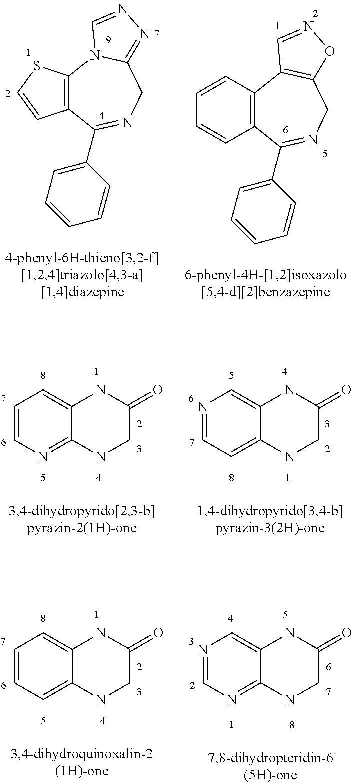 [SMILES: c1ccc(C2=NCc3nncn3-c3sccc32)cc1 ; c1ccc(C2=NCc3oncc3-c3ccccc32)cc1 ; O=C1CNc2ncccc2N1 ; O=C1CNc2ccncc2N1 ; O=C1CNc2ccccc2N1 ; O=C1CNc2ncncc2N1]

Based on the chemical structure, only very few types of BRD4 inhibitors have been described to date (Chun-Wa Chung et al., Progress in Medicinal Chemistry 2012, 51, 1-55).

The first published BRD4 inhibitors were diazepines. For example, phenylthienotriazolo-1,4-diazepines (4-phenyl-6H-thieno[3,2-f][1,2,4]triazolo[4,3-a][1,4]diazepines) are described in WO2009/084693 (Mitsubishi Tanabe Pharma Corporation) and as compound JQ1 in WO2011/143669 (Dana Farber Cancer Institute). The replacement of the thieno unit with a benzo unit likewise leads to active inhibitors (J. Med. Chem. 2011, 54, 3827-3838; E. Nicodeme et al., Nature 2010, 468, 1119). Further 4-phenyl-6H-thieno[3,2-f][1,2,4]triazolo[4,3-a][1,4]diazepines and related compounds having alternative rings as a fusion partner rather than the benzo unit are claimed generically or described explicitly in WO2012/075456 (Constellation Pharmaceuticals).

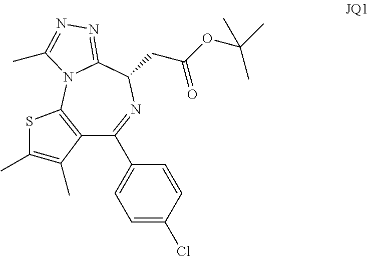 [SMILES: Cc1sc2c(c1C)C(c1ccc(Cl)cc1)=N[C@@H](CC(=O)OC(C)(C)C)c1nnc(C)n1-2]

Azepines as BRD4 inhibitors have recently been described in WO2012/075383 (Constellation Pharmaceuticals). This application relates to 6-substituted 4H-isoxazolo[5,4-d][2]benzazepines and 4H-isoxazolo[3,4-d][2]benzazepines, including those compounds which have optionally substituted phenyl at position 6, and also to analogues with alternative heterocyclic fusion partners rather than the benzo moiety, for example thieno- or pyridoazepines. Another structural class of BRD4 inhibitors described is that of 7-isoxazoloquinolines and related quinolone derivatives (Bioorganic & Medicinal Chemistry Letters 22 (2012) 2963-2967). WO2011/054845 (GlaxoSmithKline) describes further benzodiazepines as BRD4 inhibitors.

Further BRD4 inhibitors are also described by the applicant in the following applications:

WO2013/030150-6H-thieno[3,2-f][1,2,4]triazolo[4,3-a][4,3-a][1,4]diazepines,
WO2014/128111-4-substituted pyrrolo- and pyrazolo diazepines,
WO2014/128070-pyrrolo- and pyrazolodiazepines,
WO2014/026997-2,3-benzodiazepines,
WO2014/048945-5-aryltriazoloazepines,
WO2014/095774-dihydropyridopyrazinones,
WO2014/202578-2,3-benzodiazepines,
WO2014/128067-bicyclic and spirocyclic substituted 2,3-benzodiazepines,
WO2015/004075-dihydroquinoxalinones and dihydropyridopyrazinones,
and
WO2014/095775-dihydroquinoxalinones.

The applicant of the application WO 2015/011084 discloses dihydropyridopyazinone derivatives as dual inhibitors of BRD4 and polo-like kinase 1 (PLK-1).

The compounds according to the invention, in contrast, are substituted 3,4-dihydropyrido[2,3-b]pyrazin-2(1H)-one derivatives with a meta-substituted aromatic amino or ether group, which differ structurally in various ways from the chemotypes of BRD4 inhibitors discussed above. Because of the significant structural differences, it was not to be expected that the compounds claimed here would also have BRD4-inhibitory action. It is therefore surprising that the compounds according to the invention have good inhibitory action in spite of the considerable structural differences.

Some documents include compounds which are structurally similar but are aimed at completely different mechanisms of action, and in some cases also other indications. Dihydropyridopyrazinones and related bicyclic systems have been described in a series of patent applications.

WO 2013/071217 (OSI Pharmaceuticals) discloses mainly 7,8-dihydropteridin-6(5H)-ones, but also 1,4-dihydropyrido[3,4-b]pyrazin-3(2H)-one derivatives as inhibitors of kinases, in particular of RSK-1 and RSK-2, as medicaments, inter alia for the treatment of various neoplastic disorders. However, the compounds disclosed therein differ from the compounds according to the invention inter alia in the obligatory aromatic substitution at the nitrogen atom directly adjacent to the oxo group (N-5 in the dihydropteridones, or N-4 in the dihydropyrido[3,4-b]pyrazinones).

WO 2010/085570 (Takeda Pharmaceutical Company) describes inhibitors of poly-ADP-ribose polymerase (PARP) which are derived from a series of bi- and tricyclic skeletons, and which include 3,4-dihydropyrido[2,3-b]pyrazin-2(1H)-one derivatives, as medicaments for treatment of various diseases. The exemplary compounds disclosed therein differ from the inventive compounds, for example, by the type and position of the substitution on the pyrido moiety of the dihydropyridopyrazinone skeleton.

WO 2006/005510 (Boehringer Ingelheim) describes 1,4-dihydropyrido[3,4-b]pyrazin-3(2H)-one derivatives as inhibitors of PLK-1 for treatment of hyperproliferative disorders. The position of the pyrido nitrogen distinguishes the substances disclosed in that publication from the inventive compounds.

WO 2008/117061 (Sterix Ltd) describes a series of bicyclic chemotypes as inhibitors of steroid sulphatase, inter alia for inhibiting the growth of tumours.

US 2006/0019961 (P. E. Mahaney et al.) describes substituted 3,4-dihydroquinoxalin-2(1H)-one derivatives as modulators of the oestrogen receptor for treatment of various inflammation disorders, cardiovascular disorders and autoimmune disorders.

WO 2006/050054, WO 2007/134169 and US 2009/0264384 (Nuada LLC) describe a series of bicyclic chemotypes as inhibitors of tumour necrosis factor alpha (TNF-α) and various isoforms of phosphodiesterase for treatment of inflammation disorders among others.

WO 2012/088314 (Agios Pharmaceuticals) discloses a series of bicyclic chemotypes as modulators of pyruvate kinase M2.

WO 2003/020722 and WO 2004/076454 (Boehringer Ingelheim) disclose 7,8-dihydropteridin-6(5H)-ones as inhibitors of specific cell cycle kinases for treatment of hyperproliferative disorders.

WO 2006/018182 (Boehringer Ingelheim) describes pharmaceutical preparations of 7,8-dihydropteridin-6(5H)-ones in combination inter alia with various cytostatics for treatment of neoplastic disorders.

WO 2006/018185 (Boehringer Ingelheim) describes the use of 7,8-dihydropteridin-6(5H)-ones for treatment of various neoplastic disorders.

WO 2011/101369 (Boehringer Ingelheim), WO 2011/113293 (Jiangsu Hengrui Medicine), WO 2009/141575 (Chroma Therapeutics), WO 2009/071480 (Nerviano Medical Sciences) and also WO 2006/021378, WO 2006/021379 and WO 2006/021548 (likewise Boehringer Ingelheim) disclose further 7,8-dihydropteridin-6(5H)-one derivatives as inhibitors of PLK-1 for treating hyperproliferative disorders.

U.S. Pat. No. 6,369,057 describes various quinoxaline and quinoxalinone derivatives as antivirally active compounds; EP 0657166 and EP 728481 describe combinations of such compounds with nucleosides or protease inhibitors having antiviral action.

WO 2007/022638 (Methylgene Inc.) discloses, in quite general terms, HDAC inhibitors of several chemotypes, but the structures of the example compounds disclosed differ distinctly from the compounds of the present invention.

WO 1999/050254 (Pfizer) describes a series of bicyclic chemotypes as inhibitors of serine proteases for antithrombotic therapy, but these compounds differ distinctly by the type and position of the substituents from the compounds according to the invention.

Some 3,4-dihydroquinoxalin-2(1H)-one derivatives substituted at C-6 by an aromatic amino group, in which the phenyl group is in turn substituted by a para-amide group (corresponding to 2-oxo-1,2,3,4-tetrahydroquinoxaline derivatives), are indexed by Chemical Abstracts as “Chemical Library” substances without a literature reference [see 4-{[(3R)-4-cyclopentyl-3-ethyl-1-methyl-2-oxo-1,2,3,4-tetrahydroquinoxalin-6-yl]amino}-3-methoxy-N-[2-methyl-1-(pyrrolidin-1-yl)propan-2-yl]benzamide, CAS Registry No. 1026451-60-4, N-(1-benzylpiperidin-4-yl)-4-{[(3R)-4-cyclopentyl-1,3-dimethyl-2-oxo-1,2,3,4-tetrahydroquinoxalin-6-yl]amino}-3-methoxybenzamide, CAS Registry No. 1026961-36-3,4-{[(3R)-4-cyclohexyl-1,3-dimethyl-2-oxo-1,2,3,4-tetrahydroquinoxalin-6-yl]amino}-N-[1-(dimethylamino)-2-methylpropan-2-yl]-3-methoxybenzamide, CAS Registry No. 1025882-57-8]. No therapeutic use for these compounds has been described to date.

Nevertheless, there is still a great need for active compounds for prophylaxis and treatment of disorders, especially of hyperproliferative disorders, and very particularly of neoplastic disorders.

It has now been found that compounds of the general formula (I)

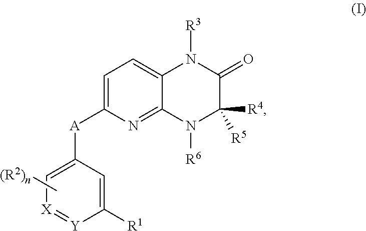

in which
A is —NH—, —N(C₁-C₃-alkyl)- or —O—,
X is —N—, —CH— or —CR²—,
Y is —N—, —CH— or —CR²—,
n is 0, 1 or 2,
R¹ is halogen, C₁-C₄-alkyl-, halo-C₁-C₄-alkyl-, cyano, —S(═O)₂R⁷,
   —S(═O)(═NR⁸)R⁹, —C(═O)R⁷ or —NR¹⁰R¹¹,
   or
   is phenyl- which is unsubstituted or is mono- or disubstituted identically or differently by halogen, cyano, C₁-C₄-alkyl-, C₂-C₄-alkenyl-,
   C₂-C₄-alkynyl-, halo-C₁-C₄-alkyl-, C₁-C₄-alkoxy-, halo-C₁-C₄-alkoxy-, C₁-C₄-alkylthio-, halo-C₁-C₄-alkylthio-, —NR¹⁰R¹¹, —C(═O)OR¹²,
   —C(═O)NR¹⁰R¹¹, —C(═O)R¹², —S(═O)₂R¹², —S(═O)₂NR¹⁰R¹¹,
   or
   is oxazolin-2-yl which is unsubstituted or is mono- or disubstituted identically or differently by C₁-C₃-alkyl,
R² is hydrogen, hydroxyl, halogen, cyano, nitro, C₁-C₃-alkyl-, C₂-C₄-alkenyl-, C₂-C₄-alkynyl-, halo-C₁-C₄-alkyl-, C₁-C₄-alkoxy-, halo-C₁-C₄-alkoxy-, C₁-C₄-alkylthio-, halo-C₁-C₄-alkylthio-, phenyl- or phenoxy-,
   where phenyl- and the phenyl-present in phenoxy- are unsubstituted or
   are mono-, di- or trisubstituted identically or differently by halogen, cyano, C₁-C₃-alkyl and C₁-C₃-alkoxy, and
   if n is 2, R² may be identical or different,
   or
R¹ and R² together are a group *—S(═O)₂—CH₂—CH₂—** or
   *—S(═O)₂—CH₂—CH₂—CH₂—**, where “*” signifies the point of attachment of R¹ to the phenyl ring or 6-membered heteroaryl ring to which R¹ is bonded, and where “**” signifies a carbon atom of this ring adjacent to this point of attachment,
R³ is methyl- or ethyl-,
R⁴ is hydrogen or C₁-C₃-alkyl,
R⁵ is hydrogen or C₁-C₃-alkyl,
   or
R⁴ and R⁵ together are C₂-C₅-alkylene,
R⁶ is C₁-C₆-alkyl which is unsubstituted or monosubstituted by
   C₁-C₃-alkoxy-, phenyl-, C₃-C₈-cycloalkyl-, or 4- to 8-membered heterocycloalkyl-, where phenyl- for its part is unsubstituted or is mono-, di- or trisubstituted identically or differently by halogen, cyano, C₁-C₄-alkyl, C₂-C₄-alkenyl, C₂-C₄-alkynyl, C₁-C₄-alkoxy, halo-C₁-C₄-alkyl or halo-C₁-C₄-alkoxy, and where C₃-C₈-cycloalkyl- or 4- to 8-membered heterocycloalkyl- for their part are unsubstituted or are mono- or disubstituted identically or differently by C₁-C₃-alkyl-,
   or
   is C₃-C₈-cycloalkyl- or 4- to 8-membered heterocycloalkyl-, which are unsubstituted or are mono- or disubstituted identically or differently by C₁-C₃-alkyl-, halo-C₁-C₃-alkyl- or C₁-C₄-alkoxycarbonyl-,
   or
   is phenyl or 5- to 6-membered heteroaryl- which are unsubstituted or are mono- or disubstituted identically or differently by halogen,
   C₁-C₃-alkyl- or 4- to 8-membered heterocycloalkyl-,
      where the 4- to 8-membered heterocycloalkyl- for its part is unsubstituted or mono- or disubstituted identically or differently by
   C₁-C₃-alkyl- or C₁-C₄-alkoxycarbonyl-,
R⁷ is C₁-C₆-alkyl- which is unsubstituted or is monosubstituted by cyano, C₁-C₃-alkoxy-, C₁-C₃-alkylamino-, phenyl-, C₃-C₈-cycloalkyl- or 4- to 8-membered heterocycloalkyl-,
   where phenyl- for its part is unsubstituted or is mono-, di- or trisubstituted identically or differently by halogen, cyano, C₁-C₄-alkyl-, C₂-C₄-alkenyl-, C₂-C₄-alkynyl-, C₁-C₄-alkoxy-, halo-C₁-C₄-alkyl- or halo-C₁-C₄-alkoxy-, and where C₃-C₈-cycloalkyl- or 4- to 8-membered heterocycloalkyl- for their part are unsubstituted or are mono- or disubstituted identically or differently by C₁-C₃-alkyl-,
   or
   is halo-C₁-C₄-alkyl-,
   or
   is C₂-C₄-alkenyl- or C₂-C₄-alkynyl-,
   or
   is C₃-C₈-cycloalkyl- or 4- to 8-membered heterocycloalkyl-, which are unsubstituted or are mono- or disubstituted identically or differently by C₁-C₃-alkyl- or C₁-C₄-alkoxycarbonyl-, with the proviso that the
   4- to 8-membered heterocycloalkyl- is not bonded via a nitrogen atom to the carbonyl or sulphonyl group in R¹,
R⁸ is hydrogen, cyano, C₁-C₆-alkyl-, C₃-C₈-cycloalkyl- or —C(═O)OR¹²,
R⁹ is C₁-C₆-alkyl or C₃-C₈-cycloalkyl,
R¹⁰ and R¹¹ are each independently hydrogen or are unsubstituted C₁-C₃-alkyl- or C₁-C₃-alkyl-mono- or disubstituted identically or differently by hydroxyl, oxo, C₁-C₃-alkoxy-, or are fluoro-C₁-C₃-alkyl or 4- to 8-membered heterocycloalkyl-,
   where the 4- to 8-membered heterocycloalkyl- for its part is unsubstituted or mono- or disubstituted identically or differently by (C₁-C₃)-alkyl-,
   or
R¹⁰ and R¹¹ together with the nitrogen atom to which they are bonded are 4- to 8-membered heterocycloalkyl-, which is unsubstituted or is mono- or disubstituted identically or differently by hydroxyl, fluorine, oxo, cyano, C₁-C₃-alkyl-, fluoro-C₁-C₃-alkyl-, C₃-C₆-cycloalkyl-, cyclopropylmethyl-, C₁-C₃-alkylcarbonyl- or C₁-C₄-alkoxycarbonyl-, and
R¹² is C₁-C₆-alkyl- or phenyl-C₁-C₃-alkyl-,
   and diastereomers, racemates, polymorphs and physiologically acceptable salts thereof, surprisingly inhibit the interaction between BRD4 and an acetylated histone 4 peptide and thus inhibit the growth of cancer and tumour cells.

Preference is given to those compounds of the general formula (I) in which
A is —NH— or —N(methyl)-,
X is —N— or —CH—,
Y is —N— or —CH—,
n is 0, 1 or 2,
R¹ is C₁-C₃-alkyl-, fluoro-C₁-C₃-alkyl-, —S(═O)₂R⁷, —S(═O)(═NR⁸)R⁹ or —NR¹⁰R¹¹,
   or
   is phenyl- which is unsubstituted or is mono-, di- or trisubstituted identically or differently by halogen, cyano, C₁-C₃-alkyl-, trifluoromethyl-,
   C₁-C₃-alkoxy-, trifluoromethoxy- or —NR¹⁰R¹¹,
   or
   is oxazolin-2-yl- which is unsubstituted or is mono- or disubstituted identically or differently by C₁-C₃-alkyl-,
R² is hydrogen, hydroxyl, fluorine, chlorine, cyano, methyl-, ethyl-, methoxy-, ethoxy-, trifluoromethoxy- or phenoxy-, where the phenyl-present in phenoxy- is unsubstituted or is mono- or disubstituted identically or differently by fluorine, chlorine, bromine, cyano, methyl- or methoxy-, and,
   if n is 2, R² may be identical or different,
   or
R¹ and R² together are a group *—S(═O)₂—CH₂—CH₂—** or
   *—S(═O)₂—CH₂—CH₂—CH₂—**, where “*” signifies the point of attachment of R¹ to the phenyl ring or 6-membered heteroaryl ring to which R¹ is bonded, and where “**” signifies a carbon atom of this ring adjacent to this point of attachment,
R³ is methyl- or ethyl-,
R⁴ is hydrogen, methyl- or ethyl-,
R⁵ is hydrogen, methyl- or ethyl-,
R⁶ is C₂-C₅-alkyl which is unsubstituted,
   or
   is methyl- or ethyl- which is monosubstituted by C₁-C₃-alkoxy-, phenyl- or 4- to 8-membered heterocycloalkyl-,
      where phenyl- for its part is unsubstituted or is mono-, di- or trisubstituted identically or differently by fluorine, chlorine, bromine, cyano, C₁-C₃-alkyl- or C₁-C₃-alkoxy-, and
      where the 4- to 8-membered heterocycloalkyl- for its part is unsubstituted or is mono- or disubstituted by methyl-,
   or
   is C₃-C₈-cycloalkyl- or 4- to 8-membered heterocycloalkyl-, which are unsubstituted or are mono- or disubstituted identically or differently by C₁-C₃-alkyl-, fluoro-C₁-C₃-alkyl- or C₁-C₄-alkoxycarbonyl-,
   or
   is phenyl or 5- to 6-membered heteroaryl- which are unsubstituted or are mono- or disubstituted identically or differently by fluorine, chlorine, methyl- or 6-membered heterocycloalkyl-,
      in which the 6-membered heterocycloalkyl- for its part is unsubstituted or is monosubstituted by methyl- or tert-butoxycarbonyl-,
R⁷ is C₁-C₆-alkyl- which is unsubstituted or is monosubstituted by cyano, C₁-C₃-alkoxy-, C₁-C₃-alkylamino-, phenyl- or 4- to 8-membered heterocycloalkyl-,
   where phenyl- for its part is unsubstituted or is mono-, di- or trisubstituted identically or differently by fluorine, chlorine, bromine, cyano,
   C₁-C₃-alkyl-, C₁-C₃-alkoxy-, and
   where the 4- to 8-membered heterocycloalkyl- for its part is unsubstituted or mono- or disubstituted identically or differently by
   (C₁-C₃)-alkyl,
   or
   is fluoro-C₁-C₃-alkyl-,
   or
   is C₃-C₄-alkenyl- or C₃-C₄-alkynyl-,
   or
   is C₃-C₈-cycloalkyl- or 4- to 8-membered heterocycloalkyl-, which are unsubstituted or are mono- or disubstituted identically or differently by C₁-C₃-alkyl- or C₁-C₄-alkoxycarbonyl-, with the proviso that the
   4- to 8-membered heterocycloalkyl- is not bonded via a nitrogen atom to the sulphonyl group in R¹,
R⁸ is hydrogen, cyano, C₁-C₄-alkyl-, C₃-C₆-cycloalkyl- or —C(═O)OR¹²
R⁹ is (C₁-C₄)-alkyl,
R¹⁰ and R¹¹ are each independently hydrogen or are unsubstituted C₁-C₃-alkyl or C₁-C₃-alkyl monosubstituted by hydroxyl or oxo or are 5- to 6-membered heterocycloalkyl-,
   where the 5- to 6-membered heterocycloalkyl- for its part is unsubstituted or mono- or disubstituted identically or differently by (C₁-C₃)-alkyl,
   or
R¹⁰ and R¹¹ together with the nitrogen atom to which they are bonded are
   4- to 7-membered heterocycloalkyl- which is unsubstituted or mono- or disubstituted identically or differently by hydroxyl, fluorine, oxo, C₁-C₃-alkyl-, fluoro-C₁-C₃-alkyl-, cyclopropyl-, cyclopropylmethyl-, acetyl- or tert-butoxycarbonyl-, and
R¹² is C₁-C₄-alkyl- or benzyl-,
   and diastereomers, racemates, polymorphs and physiologically acceptable salts thereof.

Particular preference is given to those compounds of the general formula (I) in which
A is —NH—,
X is —CH—,
Y is —N— or —CH—,
n is 0 or 1,
R¹ is C₁-C₂-alkyl-, fluoro-C₁-C₂-alkyl-, —S(═O)₂R⁷, —S(═O)(═NR⁸)R⁹ or —NR¹⁰R¹¹,
   or
   is phenyl-, which is unsubstituted or is mono- or disubstituted identically or differently by fluorine, chlorine, bromine, cyano, methyl-, trifluoromethyl- or methoxy-,
   or
   is oxazolin-2-yl which is unsubstituted or is mono- or disubstituted by methyl-,
R² is hydrogen, fluorine, chlorine, methyl-, methoxy-, trifluoromethoxy- or phenoxy-, where the phenyl-present in phenoxy- is unsubstituted or is monosubstituted by fluorine or chlorine,
   or
R¹ and R² together are a group *—S(═O)₂—CH₂—CH₂—**, where “*” signifies the point of attachment of R¹ to the phenyl ring or pyridine ring to which R¹ is bonded, and where “**” signifies a carbon atom of this ring adjacent to this point of attachment,
R³ is methyl-,
R⁴ is methyl- or ethyl-,
R⁵ is hydrogen,
R⁶ is (C₃-C₅)-alkyl,
   or
   is methyl- which is monosubstituted by phenyl- or 4- to 6-membered heterocycloalkyl-,
      where phenyl- for its part is unsubstituted or is mono- or disubstituted identically or differently by fluorine, chlorine, cyano, methyl- or methoxy-, and where the 4- to 6-membered heterocycloalkyl- for its part is unsubstituted or is monosubstituted by methyl-,
   or
   is C₃-C₈-cycloalkyl- or 4- to 6-membered heterocycloalkyl-, which are unsubstituted or are mono- or disubstituted identically or differently by C₁-C₃-alkyl-, fluoro-C₁-C₃-alkyl- or C₁-C₄-alkoxycarbonyl-,
   or
   is phenyl-, which is unsubstituted or is mono- or disubstituted identically or differently by fluorine, chlorine or methyl-,
R⁷ is C₁-C₄-alkyl- which is unsubstituted or is monosubstituted by cyano, phenyl- or 5- to 6-membered heterocycloalkyl-,
   where phenyl- for its part is unsubstituted or is mono- or disubstituted identically or differently by fluorine, chlorine, cyano, methyl-, methoxy-, and where the 5- to 6-membered heterocycloalkyl- for its part is unsubstituted or is monosubstituted by C₁-C₃-alkyl-,
   or
   is fluoro-C₁-C₂-alkyl-,
   or
   is C₃-C₄-alkenyl-,
   or
   is C₃-C₆-cycloalkyl- or 5- to 6-membered heterocycloalkyl-, with the proviso that the
   5- to 6-membered heterocycloalkyl- is not bonded via a nitrogen atom to the sulphonyl group in R¹,
R⁸ is hydrogen, cyano, C₁-C₃-alkyl- or C₁-C₃-alkoxycarbonyl-,
R⁹ is C₁-C₃-alkyl-, and
R¹⁰ and R¹¹ are each independently hydrogen or C₁-C₃-alkyl-,
   or
R¹⁰ and R¹¹ together with the nitrogen atom to which they are bonded are
   4- to 7-membered heterocycloalkyl-, which is unsubstituted or is monosubstituted by oxo, C₁-C₃-alkyl-, cyclopropyl-, cyclopropylmethyl-, acetyl- or tert-butoxycarbonyl-,
      and diastereomers, racemates, polymorphs and physiologically acceptable salts thereof.

Very particular preference is given to those compounds of the general formula (I) in which
A is —NH—,
X is —CH—,
Y is —N— or —CH—,
n is 0 or 1,
R¹ is methyl-, trifluoromethyl-, —S(═O)₂—R⁷ or —NR¹⁰R¹¹,
   or
   is phenyl-, which is unsubstituted or is monosubstituted by fluorine, chlorine, cyano, methyl-, methoxy-,
R² is hydrogen, methyl-, methoxy-, trifluoromethoxy-, phenoxy- or para-fluorophenoxy-,
   or
R¹ and R² together are a group *—S(═O)₂—CH₂—CH₂—**, where “*” signifies the point of attachment of R¹ to the phenyl ring or pyridine ring to which R¹ is bonded, and where “**” signifies a carbon atom of this ring adjacent to this point of attachment,
R³ is methyl-,
R⁴ is methyl-,
R⁵ is hydrogen,
R⁶ is isopropyl-,
   or
   is cycloheptyl-,
   or
   is tetrahydropyranyl- or piperidinyl-, which are unsubstituted or are monosubstituted by methyl-, 2,2-difluoroethyl-, 2,2,2-trifluoroethyl-, 3,3,3-trifluoropropyl- or tert-butoxycarbonyl-,
   or
   is phenyl-, which is unsubstituted or is mono- or disubstituted identically or differently by fluorine, chlorine or methyl-,
R⁷ is C₁-C₃-alkyl-, trifluoromethyl-, allyl-, C₃-C₄-cycloalkyl- or is tetrahydropyranyl-, and
R¹⁰ and R¹¹ together with the nitrogen atom to which they are bonded are
   5- to 6-membered heterocycloalkyl- which is unsubstituted or is monosubstituted by C₁-C₃-alkyl-,
      and diastereomers, racemates, polymorphs and physiologically acceptable salts thereof.

Exceptional preference is given to those compounds of the general formula (I) in which
A is —NH—,
X is —CH—,
Y is —N— or —CH—,
n is 0 or 1,
R¹ is methyl-, trifluoromethyl-, —S(═O)₂—R⁷, —NR¹⁰R¹¹ or is para-cyanophenyl-,
R² is hydrogen, methyl-, methoxy-, trifluoromethoxy-, phenoxy- or para-fluorophenoxy-,
   or
R¹ and R² together with the phenyl ring to which they are bonded are

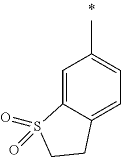

in which “*” signifies the point of attachment to the rest of the molecule,
R³ is methyl-,
R⁴ is methyl-,
R⁵ is hydrogen,
R⁶ is isopropyl-,
   or
   is cycloheptyl-,
   or is tetrahydropyran-4-yl- or piperidin-4-yl-, where piperidin-4-yl- is unsubstituted or is monosubstituted on the nitrogen by methyl-, 2,2-difluoroethyl-, 2,2,2-trifluoroethyl-, 3,3,3-trifluoropropyl- or
   tert-butoxycarbonyl-,
   or
   is phenyl-,
R⁷ is methyl-, ethyl-, isopropyl-, trifluoromethyl-, allyl-, cyclopropyl-, cyclobutyl- or is tetrahydropyran-4-yl-, and
R¹⁰ and R¹¹ together with the nitrogen atom to which they are bonded are N-methylpiperazinyl-,
   and diastereomers, racemates, polymorphs and physiologically acceptable salts thereof.

The present invention relates further to compounds of the general formula (I)

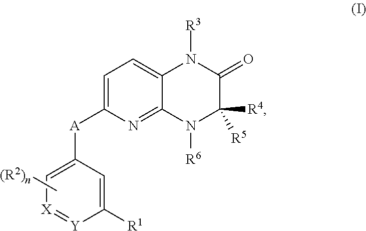

in which
A is —NH—, —N(C₁-C₃-alkyl)- or —O—,
X is —N—, —CH— or —CR²—,
Y is —N—, —CH— or —CR²—,
n is 0, 1 or 2,
R¹ is halogen, cyano, —S(═O)₂R⁷, —S(═O)(═NR⁸)R⁹, —C(═O)R⁷ or —NR¹⁰R¹¹,
   or
   is phenyl-, which is unsubstituted or is mono-, di- or trisubstitured, identically or differently, by halogen, cyano, C₁-C₄-alkyl-, C₂-C₄-alkenyl-, C₂-C₄-alkynyl-, halo-C₁-C₄-alkyl-, C₁-C₄-alkoxy-, halo-C₁-C₄-alkoxy-, C₁-C₄-alkylthio-, halo-C₁-C₄-alkylthio-, —NR¹⁰R¹¹, —C(═O)OR¹², —C(═O)N¹⁰R¹¹, —C(═O)R¹², —S(═O)₂R¹², —S(═O)₂NR¹⁰R¹¹,
   or
   is oxazolin-2-yl- which is unsubstituted or is mono- or disubstituted identically or differently by C₁-C₃-alkyl-,
R² is hydrogen, hydroxyl, halogen, cyano, C₁-C₃-alkyl-, C₂-C₄-alkenyl-, C₂-C₄-alkynyl-, halo-C₁-C₄-alkyl-, C₁-C₄-alkoxy-, halo-C₁-C₄-alkoxy-, C₁-C₄-alkylthio- or halo-C₁-C₄-alkylthio-, and, if n is 2, R² may be the same or different,
   or
R¹ and R² together are a group *—S(═O)₂—CH₂—CH₂—** or *—S(═O)₂—CH₂—CH₂—CH₂—**, where “*” signifies the point of attachment of R¹ to the phenyl ring or 6-membered heteroaryl ring to which R¹ is bonded, and where “**” signifies a carbon atom of this ring adjacent to this point of attachment,
R³ is methyl- or ethyl-,
R⁴ is hydrogen or C₁-C₃-alkyl-,
R⁵ is hydrogen or C₁-C₃-alkyl-,
   or
R⁴ and R⁵ together are C₂-C₅-alkylene,
R⁶ is C₁-C₆-alkyl- which is unsubstituted or is monosubstituted by C₁-C₃-alkoxy-, phenyl-, C₃-C₈-cycloalkyl- or 4- to 8-membered heterocycloalkyl-,
   where phenyl- for its part is unsubstituted or is mono-, di- or trisubstituted identically or differently by halogen, cyano, C₁-C₄-alkyl-, C₂-C₄-alkenyl-, C₂-C₄-alkynyl-, C₁-C₄-alkoxy-, halo-C₁-C₄-alkyl- or halo-C₁-C₄-alkoxy-, and where C₃-C₈-cycloalkyl- or 4- to 8-membered heterocycloalkyl- for their part are unsubstituted or are mono- or disubstituted identically or differently by C₁-C₃-alkyl-,
   or
   is C₃-C₈-cycloalkyl- or 4- to 8-membered heterocycloalkyl-, which are unsubstituted or are mono- or disubstituted identically or differently by C₁-C₃-alkyl- or C₁-C₄-alkoxycarbonyl-,
   or
   is phenyl or 5- to 6-membered heteroaryl- which are unsubstituted or are mono- or disubstituted identically or differently by halogen, C₁-C₃-alkyl- or 4- to 8-membered heterocycloalkyl-,
      where the 4- to 8-membered heterocycloalkyl- for its part is unsubstituted or mono- or disubstituted identically or differently by C₁-C₃-alkyl or C₁-C₄-alkoxycarbonyl-,
R⁷ is C₁-C₆-alkyl- which is unsubstituted or is monosubstituted by cyano, C₁-C₃-alkoxy-, C₁-C₃-alkylamino-, phenyl-, C₃-C₈-cycloalkyl- or 4- to 8-membered heterocycloalkyl-,
   where phenyl- for its part is unsubstituted or is mono-, di- or trisubstituted identically or differently by halogen, cyano, C₁-C₄-alkyl-, C₂-C₄-alkenyl-, C₂-C₄-alkynyl-, C₁-C₄-alkoxy-, halo-C₁-C₄-alkyl- or halo-C₁-C₄-alkoxy-, and where C₃-C₈-cycloalkyl- or 4- to 8-membered heterocycloalkyl- for their part are unsubstituted or are mono- or disubstituted identically or differently by C₁-C₃-alkyl-,
   or
   is halo-C₁-C₄-alkyl-,
   or
   is C₃-C₈-cycloalkyl- or 4- to 8-membered heterocycloalkyl- which are unsubstituted or are mono- or disubstituted identically or differently by C₁-C₃-alkyl- or C₁-C₄-alkoxycarbonyl-, with the proviso that the 4- to 8-membered heterocycloalkyl- is not bonded via a nitrogen atom to the carbonyl or sulphonyl group in R¹,
R⁸ is cyano, C₁-C₆-alkyl-, C₃-C₈-cycloalkyl- or —C(═O)OR¹²,
R⁹ is C₁-C₆-alkyl- or C₃-C₈-cycloalkyl-,
R¹⁰ and R¹¹ are each independently hydrogen or are unsubstituted C₁-C₃-alkyl- or C₁-C₃-alkyl-mono- or disubstituted identically or differently by hydroxyl, oxo, C₁-C₃-alkoxy-, or are fluoro-C₁-C₃-alkyl or 4- to 8-membered heterocycloalkyl-,
   where the 4- to 8-membered heterocycloalkyl- for its part is unsubstituted or mono- or disubstituted identically or differently by C₁-C₃-alkyl-,
   or
R¹⁰ and R¹¹ together with the nitrogen atom to which they are attached are 4- to 8-membered heterocycloalkyl-, which is unsubstituted or is mono- or disubstitued, identically or differently, by hydroxyl, fluorine, oxo, cyano, C₁-C₃-alkyl-, fluoro-C₁-C₃-alkyl-, C₃-C₆-cycloalkyl-, cyclopropylmethyl-, C₁-C₃-alkylcarbonyl- or C₁-C₄-alkoxycarbonyl-,
R¹² is C₁-C₆-alkyl- or phenyl-C₁-C₃-alkyl-,
   and diastereomers, racemates, polymorphs and physiologically acceptable salts thereof.

Of major interest, furthermore, are those compounds of the general formula I in which
A is —NH— or —N(methyl)-,
X is —N— or —CH—,
Y is —CH—,
n is 0, 1 or 2,
R¹ is —S(═O)₂R⁷, —S(═O)(═NR⁸)R⁹ or —NR¹⁰R¹¹,
   or
   is phenyl- which is unsubstituted or is mono-, di- or trisubstituted identically or differently by halogen, cyano, C₁-C₃-alkyl-, trifluoromethyl-, C₁-C₃-alkoxy-, trifluoromethoxy- and —NR¹⁰R¹¹,
   or
   is oxazolin-2-yl- which is unsubstituted or is mono- or disubstituted identically or differently by C₁-C₃-alkyl-,
R² is hydrogen, hydroxyl, fluorine, chlorine, cyano, methyl-, methoxy-, ethyl- or ethoxy-, and if n is 2, R² may be the same or different,
   or
R¹ and R² together are a group *—S(═O)₂—CH₂—CH₂—** or *—S(═O)₂—CH₂—CH₂—CH₂—**, where “*” signifies the point of attachment of R¹ to the phenyl ring or 6-membered heteroaryl ring to which R¹ is bonded, and where “**” signifies a carbon atom of this ring adjacent to this point of attachment,
R³ is methyl- or ethyl-,
R⁴ is hydrogen, methyl- or ethyl-,
R⁵ is hydrogen, methyl- or ethyl-,
R⁶ is (C₂-C₅)-alkyl-,
   or
   is methyl- or ethyl- which is monosubstituted by C₁-C₃-alkoxy-, phenyl- or 4- to 8-membered heterocycloalkyl-,
      where phenyl- for its part is unsubstituted or is mono-, di- or trisubstituted identically or differently by fluorine, chlorine, bromine, cyano, C₁-C₃-alkyl- or C₁-C₃-alkoxy-, and
      where the 4- to 8-membered heterocycloalkyl- for its part is unsubstituted or is mono- or disubstituted by methyl-,
   or
   is C₃-C₈-cycloalkyl- or 4- to 8-membered heterocycloalkyl-, which are unsubstituted or are mono- or disubstituted identically or differently by C₁-C₃-alkyl- or C₁-C₄-alkoxycarbonyl-,
   or
   is phenyl or 5- to 6-membered heteroaryl- which are unsubstituted or are mono- or disubstituted identically or differently by fluorine, chlorine, methyl- or 6-membered heterocycloalkyl-,
      in which the 6-membered heterocycloalkyl- for its part is unsubstituted or is monosubstituted by methyl- or tert-butoxycarbonyl-,
R⁷ is C₁-C₆-alkyl- which is unsubstituted or is monosubstituted by cyano, C₁-C₃-alkoxy-, C₁-C₃-alkylamino-, phenyl- or 4- to 8-membered heterocycloalkyl-,
   where phenyl- for its part is unsubstituted or is mono-, di- or trisubstituted identically or differently by fluorine, chlorine, bromine, cyano, C₁-C₃-alkyl-, C₁-C₃-alkoxy-, and
   where the 4- to 8-membered heterocycloalkyl- for its part is unsubstituted or mono- or disubstituted identically or differently by C₁-C₃-alkyl-,
   or
   is fluoro-C₁-C₃-alkyl-,
   or
   is C₃-C₈-cycloalkyl- or 4- to 8-membered heterocycloalkyl- which are unsubstituted or are mono- or disubstituted identically or differently by C₁-C₃-alkyl- or C₁-C₄-alkoxycarbonyl-, with the proviso that the 4- to 8-membered heterocycloalkyl- is not bonded via a nitrogen atom to the carbonyl or sulphonyl group in R¹,
R⁸ is cyano, C₁-C₄-alkyl-, C₃-C₆-cycloalkyl- or —C(═O)OR¹²,
R⁹ is C₁-C₄-alkyl,
R¹⁰ and R¹ are each independently hydrogen or are unsubstituted C₁-C₃-alkyl or C₁-C₃-alkyl monosubstituted by hydroxyl or oxo or are 5- to 6-membered heterocycloalkyl-,
   where the 5- to 6-membered heterocycloalkyl- for its part is unsubstituted or mono- or disubstituted identically or differently by C₁-C₃-alkyl-,
   or
R¹⁰ and R¹¹ together with the nitrogen atom to which they are attached are 4- to 7-membered heterocycloalkyl-, which is unsubstituted or is mono- or disubstitued identically or differently by hydroxyl, fluorine, oxo, C₁-C₃-alkyl-, fluoro-C₁-C₃-alkyl-, cyclopropyl-, cyclopropylmethyl-, acetyl- or tert-butoxycarbonyl-,
R¹² is C₁-C₄-alkyl- or benzyl-,
   and diastereomers, racemates, polymorphs and physiologically acceptable salts thereof.

Of special interest are also those compounds of the general formula (I) in which
A is —NH—,
X is —CH—,
Y is —CH—,
n is 0 or 1,
R¹ is —S(═O)₂R⁷ or —S(═O)(═NR⁸)R⁹,
   or
   is phenyl-, which is unsubstituted or is mono- or disubstituted identically or differently by fluorine, chlorine, bromine, cyano, methyl-, trifluoromethyl- or methoxy-,
   or
   is oxazolin-2-yl- which is unsubstituted or is mono- or disubstituted by methyl-,
R² is hydrogen, fluorine, chlorine, methyl- or methoxy,
   or
R¹ and R² together are a group *—S(═O)₂—CH₂—CH₂—**, where “*” signifies the point of attachment of R¹ to the phenyl ring to which R¹ is bonded, and where “**” signifies a carbon atom of this ring adjacent to this point of attachment,
R³ is methyl-,
R⁴ is methyl- or ethyl-,
R⁵ is hydrogen,
R⁶ is C₃-C₅-alkyl-,
   or
   is methyl- which is monosubstituted by phenyl- or 4- to 6-membered heterocycloalkyl-,
      where phenyl- for its part is unsubstituted or is mono- or disubstituted identically or differently by fluorine, chlorine, cyano, methyl- or methoxy-, and where the 4- to 6-membered heterocycloalkyl- for its part is unsubstituted or is monosubstituted by methyl-,
   or
   is C₃-C₈-cycloalkyl- or 4- to 6-membered heterocycloalkyl-, which are unsubstituted or are mono- or disubstituted identically or differently by C₁-C₃-alkyl- or C₁-C₄-alkoxycarbonyl-,
   or
   is phenyl-, which is unsubstituted or is mono- or disubstituted identically or differently by fluorine, chlorine or methyl-,
R⁷ is C₁-C₄-alkyl- which is unsubstituted or is monosubstituted by cyano, phenyl- or 5- to 6-membered heterocycloalkyl-,
   where phenyl- for its part is unsubstituted or is mono- or disubstituted identically or differently by fluorine, chlorine, cyano, methyl-, methoxy-, and where the 5- to 6-membered heterocycloalkyl- for its part is unsubstituted or is monosubstituted by C₁-C₃-alkyl-,
   or
   is C₃-C₈-cycloalkyl,
R⁸ is cyano, C₁-C₃-alkyl- or C₁-C₃-alkoxycarbonyl-,
R⁹ is C₁-C₃-alkyl-,
   and diastereomers, racemates, polymorphs and physiologically acceptable salts thereof.

Of very special interest are also those compounds of the general formula (I) in which
A is —NH—,
X is —CH—,
Y is —CH—,
n is 0 or 1,
R¹ is —S(═O)₂R⁷ or —S(═O)(═NR⁸)R⁹,
   or
   is phenyl-, which is unsubstituted or is monosubstituted by fluorine, chlorine, cyano, methyl-, methoxy-,
R² is hydrogen, methyl- or methoxy-,
   or
R¹ and R² together are a group *—S(═O)₂—CH₂—CH₂—**, where “*” signifies the point of attachment of R¹ to the phenyl ring to which R¹ is bonded, and where “**” signifies a carbon atom of this ring adjacent to this point of attachment,
R³ is methyl-,
R⁴ is methyl-,
R⁵ is hydrogen,
R⁶ is isopropyl-,
   or
   is cycloheptyl-,
   or
   is tetrahydropyranyl- or piperidinyl-, which are unsubstituted or are monosubstituted by methyl-,
   or
   is phenyl-, which is unsubstituted or is mono- or disubstituted identically or differently by fluorine, chlorine or methyl-,
R⁷ is C₁-C₃-alkyl- or cyclopropyl-,
R⁸ is C₁-C₃-alkoxycarbonyl-,
R⁹ is C₁-C₃-alkyl-,
   and diastereomers, racemates, polymorphs and physiologically acceptable salts thereof.

Exceptionally interesting are also those compounds of the general formula (I) in which
A is —NH—,
X is —CH—,
Y is —CH—,
n is 0 or 1,
R¹ is —S(═O)₂—R⁷, —S(═O)(═NR⁸)R⁹ or is para-cyanophenyl-,
R² is hydrogen, methyl- or methoxy-,
   or
R¹ and R² together with the phenyl ring to which they are bonded are

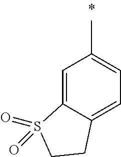

in which “*” signifies the point of attachment to the rest of the molecule,
R³ is methyl-,
R⁴ is methyl-,
R⁵ is hydrogen,
R⁶ is isopropyl-,
   or
   is cycloheptyl-,
   or is tetrahydropyran-4-yl- or N-methylpiperidin-4-yl-,
   or
   is phenyl-,
R⁷ is methyl-, isopropyl- or cyclopropyl-,
R⁸ is ethoxycarbonyl-,
R⁹ is methyl-,
   and diastereomers, racemates, polymorphs and physiologically acceptable salts thereof.

Preference is given to compounds of the general formula (I) in which A is —NH—.

Preference is given to compounds of the general formula (I) in which A is —O—.

Preference is given to compounds of the general formula (I) in which A is —NH— or is —N(C₁-C₃-alkyl)-.

Preference is given to compounds of the general formula (I) in which A is —N(C₁-C₃-alkyl)-.

Preference is given to compounds of the general formula (I) in which A is —NH— or is —N(methyl)-.

Preference is given to compounds of the general formula (I) in which A is —N(methyl)-.

Preference is given to compounds of the general formula (I) in which X is —N— or —CH—.

Preference is given to compounds of the general formula (I) in which X is —N—.

Particular preference is given to compounds of the general formula (I) in which X is —CH—.

Preference is given to compounds of the general formula (I) in which Y is —N— or —CH—.

Preference is given to compounds of the general formula (I) in which Y is —N—.

Particular preference is given to compounds of the general formula (I) in which Y is —CH—.

Preference is given to compounds of the general formula (I) in which X is —CH— and in which Y is —N— or —CH—.

Preference is given to compounds of the general formula (I) in which X is —CH— and in which Y is —N—.

Particular preference is given to compounds of the general formula (I) in which X is —CH— and in which Y is —CH—.

Preference is given to compounds of the general formula (I) in which n is the number 0 or the number 1.

Preference is given to compounds of the general formula (I) in which n is the number 0.

Preference is given to compounds of the general formula (I) in which n is the number 1.

Preference is given to compounds of the general formula (I) in which R¹ is C₁-C₃-alkyl-, fluoro-C₁-C₃-alkyl-, —S(═O)₂R⁷, —S(═O)(═NR⁸)R⁹ or —NR¹⁰R¹¹,

or
is phenyl- which is unsubstituted or is mono- or disubstituted identically or differently by halogen, cyano, C₁-C₃-alkyl-, trifluoromethyl-, C₁-C₃-alkoxy-, trifluoromethoxy- and —NR¹⁰R¹¹,
or
is oxazolin-2-yl which is unsubstituted or is mono- or disubstituted identically or differently by C₁-C₃-alkyl-.

Preference is given to compounds of the general formula (I) in which R¹ is —S(═O)₂R⁷, —S(═O)(═NR⁸)R⁹ or —NR¹⁰R¹¹,

or
is phenyl- which is unsubstituted or is mono-, di or trisubstituted identically or differently by halogen, cyano, C₁-C₃-alkyl-, trifluoromethyl-, C₁-C₃-alkoxy-, trifluoromethoxy- and —NR¹⁰R¹¹,
or
is oxazolin-2-yl which is unsubstituted or is mono- or disubstituted identically or differently by C₁-C₃-alkyl-.

Preference is given to compounds of the general formula (I) in which R¹ is C₁-C₃-alkyl-, fluoro-C₁-C₃-alkyl-, —S(═O)₂R⁷, —S(═O)(═NR⁸)R⁹ or —NR¹⁰R¹¹.

Preference is given to compounds of the general formula (I) in which R¹ is —S(═O)₂R⁷, —S(═O)(═NR⁸)R⁹ or —NR¹⁰R¹¹.

Preference is given to compounds of the general formula (I) in which R¹ is phenyl-, which is unsubstituted or is mono-, di- or trisubstituted identically or differently by halogen, cyano, C₁-C₃-alkyl-, trifluoromethyl-, C₁-C₃-alkoxy-, trifluoromethoxy- or —NR¹⁰R¹¹.

Preference is given to compounds of the general formula (I) in which R¹ is oxazolin-2-yl- which is unsubtituted or mono- or disubstituted identically or differently by C₁-C₃-alkyl-.

Particular preference is given to compounds of the general formula (I) in which R¹ is C₁-C₂-alkyl-, fluoro-C₁-C₂-alkyl-, —S(═O)₂R⁷, —S(═O)(═NR⁸)R⁹ or —NR¹⁰R¹¹,

or
is phenyl-, which is unsubstituted or is mono- or disubstituted identically or differently by fluorine, chlorine, bromine, cyano, methyl-, trifluoromethyl- or methoxy-,
or
is oxazolin-2-yl which is unsubstituted or is mono- or disubstituted by methyl-.

Particular preference is given to compounds of the general formula (I) in which R¹ is —S(═O)₂R⁷ or —S(═O)(═NR⁸)R⁹,

or
is phenyl-, which is unsubstituted or is mono- or disubstituted identically or differently by fluorine, chlorine, bromine, cyano, methyl-, trifluoromethyl- or methoxy-,
or
is oxazolin-2-yl which is unsubstituted or is mono- or disubstituted by methyl-.

Particular preference is given to compounds of the general formula (I) in which R¹ is C₁-C₂-alkyl-, fluoro-C₁-C₂-alkyl-, —S(═O)₂R⁷, —S(═O)(═NR⁸)R⁹ or —NR¹⁰R¹¹.

Particular preference is given to compounds of the general formula (I) in which R¹ is —S(═O)₂R⁷ or —S(═O)(═NR⁸)R⁹.

Particular preference is given to compounds of the general formula (I) in which R¹ is phenyl-, which is unsubstituted or mono- or disubstituted identically or differently by fluorine, chlorine, bromine, cyano, methyl-, trifluoromethyl- or methoxy-.

Particular preference is given to compounds of the general formula (I) in which R¹ is oxazolin-2-yl-which is unsubtituted or mono- or disubstituted by methyl-.

Particular preference is given to compounds of the general formula (I) in which R¹ is —S(═O)(═NR⁸)R⁹.

Very particular preference is given to compounds of the general formula (I) in which R¹

is methyl-, trifluoromethyl-, —S(═O)₂—R⁷ or —NR¹⁰R¹¹,
or
is phenyl-, which is unsubstituted or is monosubstituted by fluorine, chlorine, cyano, methyl-, methoxy-.

Very particular preference is given to compounds of the general formula (I) in which R¹

is —S(═O)₂—R⁷ or —S(═O)(═NR⁸)R⁹,
or
is phenyl-, which is unsubstituted or is monosubstituted by fluorine, chlorine, cyano, methyl-, methoxy-.

Very particular preference is given to compounds of the general formula (I) in which R¹ is methyl-, trifluoromethyl-, —S(═O)₂—R⁷ or —NR¹⁰R¹¹.

Very particular preference is given to compounds of the general formula (I) in which R¹ is —S(═O)₂—R⁷ or —S(═O)(═NR⁸)R⁹.

Very particular preference is given to compounds of the general formula (I) in which R¹ is phenyl-, which is unsubstituted or is monosubstituted by fluorine, chlorine, cyano, methyl-, methoxy-.

Exceptional preference is given to compounds of the general formula (I) in which R¹ is methyl-, trifluoromethyl-, —S(═O)₂—R⁷, —NR¹⁰R¹¹ or is para-cyanophenyl-,

Exceptional preference is given to compounds of the general formula (I) in which R¹ is —S(═O)₂R⁷, —S(═O)(═NR⁸)R⁹ or is para-cyanophenyl-.

Exceptional preference is given to compounds of the general formula (I) in which R¹ is —S(═O)₂R⁷.

Exceptional preference is given to compounds of the general formula (I) in which R¹ is —S(═O)₂—R⁷, where R⁷ is C₁-C₃-alkyl-, trifluoromethyl-, allyl-, C₃-C₄-cycloalkyl- or is tetrahydropyranyl-.

Exceptional preference is given to compounds of the general formula (I) in which R¹ is —NR¹⁰R¹¹.

Exceptional preference is given to compounds of the general formula (I) in which R¹ is —NR¹⁰R¹¹, where —NR¹⁰R¹¹ is 5- to 6-membered heterocycloalkyl- which is unsubstituted or is monosubstituted by C₁-C₃-alkyl-.

Exceptional preference is given to compounds of the general formula (I) in which R¹ is —NR¹⁰R¹¹, where —NR¹⁰R¹¹ is piperidinyl-, piperazinyl- or morpholinyl-, which is unsubstituted or is monosubstituted by methyl-.

Exceptional preference is given to compounds of the general formula (I) in which R¹ is para-cyanophenyl-.

Preference is given to compounds of the general formula (I), in which R² is hydrogen, hydroxyl, fluorine, chlorine, cyano, methyl-, methoxy-, ethyl-, ethoxy-, trifluoromethoxy- or phenoxy-, where the phenyl-present in phenoxy- is unsubstituted or is mono- or disubstituted, identically or differently, by fluorine, chlorine, bromine, cyano, methyl- or methoxy-.

Preference is given to compounds of the general formula (I) in which R² is hydrogen, hydroxyl, fluorine, chlorine, cyano, methyl-, methoxy-, ethyl- or ethoxy-.

Preference is given to compounds of the general formula (I) in which R² is C₁-C₃-alkoxy-.

Preference is given to compounds of the general formula (I) in which R² is ethoxy-.

Preference is given to compounds of the general formula (I) in which R² is hydroxyl.

Preference is given to compounds of the general formula (I) in which R² is fluorine.

Preference is given to compounds of the general formula (I) in which R² is chlorine.

Particular preference is given to compounds of the general formula (I) in which R² is hydrogen, fluorine, chlorine, methyl-, methoxy-, trifluoromethoxy- or phenoxy-, where the phenyl-present in phenoxy- is unsubstituted or is monosubstituted by fluorine or chlorine.

Particular preference is given to compounds of the general formula (I) in which R² is hydrogen, fluorine, chlorine, methyl- or methoxy-.

Particular preference is given to compounds of the general formula (I) in which R² is hydrogen, methyl-, methoxy- or trifluoromethoxy-.

Particular preference is given to compounds of the general formula (I) in which R² is hydrogen, methyl- or methoxy-.

Particular preference is given to compounds of the general formula (I) in which R² is methoxy-.

Particular preference is given to compounds of the general formula (I) in which R² is trifluoromethoxy-.

Particular preference is given to compounds of the general formula (I) in which R² is methyl-.

Particular preference is given to compounds of the general formula (I) in which R² is phenoxy-, where the phenyl-present in phenoxy- is unsubstituted or is monosubstituted by fluorine or chlorine.

Very particular preference is given to compounds of the general formula (I) in which R² is hydrogen, methyl-, methoxy- or trifluoromethoxy-, phenoxy- or para-fluorophenoxy-.

Very particular preference is given to compounds of the general formula (I) in which R² is phenoxy- or para-fluorophenoxy-.

Very particular preference is given to compounds of the general formula (I) in which R² is phenoxy-,

Very particular preference is given to compounds of the general formula (I) in which R² is para-fluorophenoxy-.

Particular preference is given to compounds of the general formula (I) in which R² represents hydrogen.

Preference is given to compounds of the general formula (I) in which R¹ and R² together are a group *—S(═O)₂—CH₂—CH₂—** or *—S(═O)₂—CH₂—CH₂—CH₂—**, where “*” signifies the point of attachment of R¹ to the phenyl ring or 6-membered heteroaryl ring to which R¹ is bonded, and where “**” signifies a carbon atom of this ring adjacent to this point of attachment.

Particular preference is given to compounds of the general formula (I) in which R¹ and R² together are a group *—S(═O)₂—CH₂—CH₂—**, where “*” signifies the point of attachment of R¹ to the phenyl ring or pyridine ring to which R¹ is bonded, and where “**” signifies a carbon atom of this ring adjacent to this point of attachment.

Very particular preference is given to compounds of the general formula (I) in which R¹ and R² together are a group *—S(═O)₂—CH₂—CH₂—**, where “*” signifies the point of attachment of R¹ to the phenyl ring to which R¹ is bonded, and where “**” signifies a carbon atom of this ring adjacent to this point of attachment.

Exceptional preference is given to compounds of the general formula (I) in which R¹ and R² together with the phenyl ring to which they are bonded are

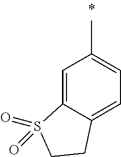

in which “*” denotes the attachment point to the rest of the molecule.

Preference is given to compounds of the general formula (I) in which R³ is methyl- or ethyl-.

Preference is given to compounds of the general formula (I) in which R³ is ethyl-.

Particular preference is given to compounds of the general formula (I) in which R³ is methyl-.

Preference is given to compounds of the general formula (I) in which R⁴ is hydrogen, methyl- or ethyl-.

Preference is given to compounds of the general formula (I) in which R⁴ is methyl- or ethyl-.

Preference is given to compounds of the general formula (I) in which R⁴ is ethyl-.

Preference is given to compounds of the general formula (I) in which R⁵ is hydrogen.

Particular preference is given to compounds of the general formula (I) in which R⁴ is methyl-.

Preference is given to compounds of the general formula (I) in which R⁴ is ethyl- and R⁵ is hydrogen.

Preference is given to compounds of the general formula (I) in which one substituent in each case from R⁴ and R⁵ is methyl- and one is hydrogen, so as to result in a racemate with respect to the stereocentre formed from R⁴, R⁵ and the carbon atom bonded to R⁴ and R⁵.

Particular preference is given to compounds of the general formula (I) in which one substituent in each case from R⁴ and R⁵ is methyl- and one is hydrogen, so as to result in an isomer mixture in which the (R) form predominates with respect to the stereocentre formed from R⁴, R⁵ and the carbon atom bonded to R⁴ and R⁵.

Particular preference is given to compounds of the general formula (I) in which R⁴ is methyl- and R⁵ is hydrogen.

Preference is given to compounds of the general formula (I) in which R⁶ is C₂-C₅-alkyl- which is unsubstituted,

or
is methyl- or ethyl-monosubstituted by C₁-C₃-alkoxy-, phenyl- or 4- to 8-membered heterocycloalkyl-
where phenyl- for its part is unsubstituted or is mono-, di- or trisubstituted identically or differently by fluorine, chlorine, bromine, cyano, C₁-C₃-alkyl- or C₁-C₃-alkoxy-, and where the 4- to 8-membered heterocycloalkyl- for its part is unsubstituted or is mono- or disubstituted by methyl-,
   or
   is C₃-C₅-cycloalkyl- or 4- to 8-membered heterocycloalkyl-, which are unsubstituted or are mono- or disubstituted identically or differently by C₁-C₃-alkyl-, fluoro-C₁-C₃-alkyl- or C₁-C₄-alkoxycarbonyl-, or
   is phenyl or 5- to 6-membered heteroaryl- which are unsubstituted or are mono- or disubstituted identically or differently by fluorine, chlorine, methyl- or 6-membered heterocycloalkyl-,
in which the 6-membered heterocycloalkyl- for its part is unsubstituted or is monosubstituted by methyl- or tert-butoxycarbonyl-.

Preference is given to compounds of the general formula (I) in which R⁶ is C₂-C₅-alkyl-,

or
is methyl- or ethyl-monosubstituted by C₁-C₃-alkoxy-, phenyl- or 4- to 8-membered heterocycloalkyl-
where phenyl- for its part is unsubstituted or is mono-, di- or trisubstituted identically or differently by fluorine, chlorine, bromine, cyano, C₁-C₃-alkyl- or C₁-C₃-alkoxy-, and where the 4- to 8-membered heterocycloalkyl- for its part is unsubstituted or is mono- or disubstituted by methyl-,
   or
   is C₃-C₈-cycloalkyl- or 4- to 8-membered heterocycloalkyl-, which are unsubstituted or are mono- or disubstituted identically or differently by C₁-C₃-alkyl- or C₁-C₄-alkoxycarbonyl-,
   or
   is phenyl or 5- to 6-membered heteroaryl- which are unsubstituted or are mono- or disubstituted identically or differently by fluorine, chlorine, methyl- or 6-membered heterocycloalkyl-,
in which the 6-membered heterocycloalkyl- for its part is unsubstituted or is monosubstituted by methyl- or tert-butoxycarbonyl-.

Preference is given to compounds of the general formula (I) in which R⁶ is C₂-C₅-alkyl- which is unsubstituted.

Preference is given to compounds of the general formula (I) in which R⁶ is methyl- or ethyl-monosubstituted by C₁-C₃-alkoxy-, phenyl- or 4- to 8-membered heterocycloalkyl-,
where phenyl- for its part is unsubstituted or is mono-, di- or trisubstituted identically or differently by fluorine, chlorine, bromine, cyano, C₁-C₃-alkyl-, C₁-C₃-alkoxy-, and where the 4- to 8-membered heterocycloalkyl- for its part is unsubstituted or mono- or disubstituted identically or differently by methyl-.

Preference is given to compounds of the general formula (I) in which R⁶ is C₃-C₈-cycloalkyl- or 4- to 8-membered heterocycloalkyl- which are unsubstituted or are mono- or disubstituted identically or differently by C₁-C₃-alkyl- or fluoro-C₁-C₃-alkyl- or C₁-C₄-alkoxycarbonyl-.

Preference is given to compounds of the general formula (I) in which R⁶ is C₃-C₈-cycloalkyl- or 4- to 8-membered heterocycloalkyl- which are unsubstituted or are mono- or disubstituted identically or differently by C₁-C₃-alkyl- or C₁-C₄-alkoxycarbonyl-.

Preference is given to compounds of the general formula (I) in which R⁶ is phenyl or 5- to 6-membered heteroaryl- which are unsubstituted or are mono- or disubstituted identically or differently by fluorine, chlorine, methyl- or 6-membered heterocycloalkyl-,
in which the 6-membered heterocycloalkyl- for its part is unsubstituted or is monosubstituted by methyl- or tert-butoxycarbonyl-.

Preference is given to compounds of the general formula (I) in which R⁶ is C₃-C₅-alkyl-, which is unsubstituted,

or
is methyl-monosubstituted by phenyl- or 4- to 6-membered heterocycloalkyl-,
where phenyl- for its part is unsubstituted or is mono- or disubstituted identically or differently by fluorine, chlorine, cyano, methyl- or methoxy-, and
where the 4- to 6-membered heterocycloalkyl- for its part is unsubstituted or is monosubstituted by methyl-,
   or
   is C₃-C₈-cycloalkyl- or 4- to 6-membered heterocycloalkyl-, which are unsubstituted or are mono- or disubstituted identically or differently by C₁-C₃-alkyl-, fluoro-C₁-C₃-alkyl- or C₁-C₄-alkoxycarbonyl-, or
   is phenyl-, which is unsubstituted or is mono- or disubstituted identically or differently by fluorine, chlorine or methyl-.

Particular preference is given to compounds of the general formula (I) in which R⁶ is C₃-C₅-alkyl-, or

is methyl monosubstituted by phenyl- or 4- to 6-membered heterocycloalkyl-,
where phenyl- for its part is unsubstituted or is mono- or disubstituted identically or differently by fluorine, chlorine, cyano, methyl- or methoxy-, and
where the 4- to 6-membered heterocycloalkyl- for its part is unsubstituted or is monosubstituted by methyl-,
   or
   is C₃-C₈-cycloalkyl- or 4- to 6-membered heterocycloalkyl-, which are unsubstituted or are mono- or disubstituted identically or differently by C₁-C₃-alkyl- or C₁-C₄-alkoxycarbonyl-,
   or
   is phenyl-, which is unsubstituted or is mono- or disubstituted identically or differently by fluorine, chlorine or methyl-.

Preference is given to compounds of the general formula (I) in which R⁶ is C₃-C₅-alkyl-, which is unsubstituted.

Particular preference is given to compounds of the general formula (I) in which R⁶ is methyl-monosubstituted by phenyl- or 4- to 6-membered heterocycloalkyl-,
where phenyl- for its part is unsubstituted or is mono- or disubstituted identically or differently by fluorine, chlorine, cyano, methyl- or methoxy-, and
where the 4- to 6-membered heterocycloalkyl- for its part is unsubstituted or is monosubstituted by methyl-.

Particular preference is given to compounds of the general formula (I) in which R⁶ is C₃-C₈-cycloalkyl- or 4- to 6-membered heterocycloalkyl-, which are unsubstituted or are mono- or disubstituted identically or differently by C₁-C₃-alkyl-, fluoro-C₁-C₃-alkyl- or C₁-C₄-alkoxycarbonyl-.

Particular preference is given to compounds of the general formula (I) in which R⁶ is is C₃-C₈-cycloalkyl- or 4- to 6-membered heterocycloalkyl-, which are unsubstituted or are mono- or disubstituted identically or differently by C₁-C₃-alkyl- or C₁-C₄-alkoxycarbonyl-.

Particular preference is given to compounds of the general formula (I) in which R⁶ is phenyl-, which is unsubstituted or mono- or disubstituted identically or differently by fluorine, chlorine or methyl-.

Very particular preference is given to compounds of the general formula (I) in which R⁶ is isopropyl-, or

is cycloheptyl-,
or
is tetrahydropyranyl- or piperidinyl-, which are unsubstituted or are monosubstituted by methyl-, 2,2-difluoroethyl-, 2,2,2-trifluoroethyl-, 3,3,3-trifluoropropyl- or
tert-butoxycarbonyl-,
or
is phenyl-, which is unsubstituted or is mono- or disubstituted identically or differently by fluorine, chlorine or methyl-.

Very particular preference is given to compounds of the general formula (I) in which R⁶ is isopropyl-, or

is cycloheptyl-,
or
is tetrahydropyranyl- or piperidinyl-, which are unsubstituted or are monosubstituted by methyl-,
or
is phenyl-, which is unsubstituted or is mono- or disubstituted identically or differently by fluorine, chlorine or methyl-.

Very particular preference is given to compounds of the general formula (I) in which R⁶ is isopropyl-.

Very particular preference is given to compounds of the general formula (I) in which R⁶ is cycloheptyl-.

Very particular preference is given to compounds of the general formula (I) in which R⁶ is tetrahydropyranyl- or piperidinyl-, which are unsubstituted or monosubstituted by methyl-, 2,2-difluoroethyl-, 2,2,2-trifluoroethyl-, 3,3,3-trifluoropropyl- or tert-butoxycarbonyl-.

Very particular preference is given to compounds of the general formula (I) in which R⁶ is tetrahydropyranyl- or piperidinyl-, which are unsubstituted or monosubstituted by methyl-.

Exceptional preference is given to compounds of the general formula (I) in which R⁶ is isopropyl-, or

is cycloheptyl-,
or
is tetrahydropyran-4-yl- or piperidin-4-yl-, where piperidin-4-yl- is unsubstituted or is monosubstituted on the nitrogen by methyl-, 2,2-difluoroethyl-, 2,2,2-trifluoroethyl-, 3,3,3-trifluoropropyl- or tert-butoxycarbonyl-,
or
is phenyl.

Exceptional preference is given to compounds of the general formula (I) in which R⁶ is isopropyl-, or

is cycloheptyl-,
or
is tetrahydropyran-4-yl- or N-methylpiperidin-4-yl-,
or
is phenyl.

Exceptional preference is given to compounds of the general formula (I) in which R⁶ is tetrahydropyran-4-yl- or piperidin-4-yl-, where piperidin-4-yl- is unsubstituted or is monosubstituted on the nitrogen by methyl-, 2,2-difluoroethyl-, 2,2,2-trifluoroethyl-, 3,3,3-trifluoropropyl- or tert-butoxycarbonyl-,

Exceptional preference is given to compounds of the general formula (I) in which R⁶ is tetrahydropyran-4-yl- or N-methylpiperidin-4-yl-.

Exceptional preference is given to compounds of the general formula (I) in which R⁶ is tetrahydropyran-4-yl.

Exceptional preference is given to compounds of the general formula (I) in which R⁶ is piperidin-4-yl-, which is unsubstituted or is monosubstituted on the nitrogen by methyl-, 2,2-difluoroethyl-, 2,2,2-trifluoroethyl-, 3,3,3-trifluoropropyl- or tert-butoxycarbonyl-.

Exceptional preference is given to compounds of the general formula (I) in which R⁶ is N-methylpiperidin-4-yl-.

Exceptional preference is given to compounds of the general formula (I) in which R⁶ is phenyl-.

Preference is given to compounds of the general formula (I) in which R⁷ is C₁-C₆-alkyl- which is unsubstituted or is monosubstituted by cyano, C₁-C₃-alkoxy-, C₁-C₃-alkylamino-, phenyl- or 4- to 8-membered heterocycloalkyl-,
where phenyl- for its part is unsubstituted or is mono-, di- or trisubstituted identically or differently by fluorine, chlorine, bromine, cyano, C₁-C₃-alkyl-, C₁-C₃-alkoxy-, and where the 4- to 8-membered heterocycloalkyl- for its part is unsubstituted or mono- or disubstituted identically or differently by C₁-C₃-alkyl-,
   or
   is fluoro-C₁-C₃-alkyl-,
   or
   is C₃-C₄-alkenyl- or C₃-C₄-alkynyl-,
   or
   is C₃-C₈-cycloalkyl- or 4- to 8-membered heterocycloalkyl-, which are unsubstituted or are mono- or disubstituted identically or differently by C₁-C₃-alkyl- or
   C₁-C₄-alkoxycarbonyl-, with the proviso that the 4- to 8-membered heterocycloalkyl- is not bonded via a nitrogen atom to the carbonyl or sulphonyl group in R¹.

Preference is given to compounds of the general formula (I) in which R⁷ is C₁-C₆-alkyl- which is unsubstituted or is monosubstituted by cyano, C₁-C₃-alkoxy-, C₁-C₃-alkylamino-, phenyl- or 4- to 8-membered heterocycloalkyl-,
where phenyl- for its part is unsubstituted or is mono-, di- or trisubstituted identically or differently by fluorine, chlorine, bromine, cyano, C₁-C₃-alkyl-, C₁-C₃-alkoxy-, and where the 4- to 8-membered heterocycloalkyl- for its part is unsubstituted or mono- or disubstituted identically or differently by C₁-C₃-alkyl-,
   or
   is fluoro-C₁-C₃-alkyl-,
   or
   is C₃-C₈-cycloalkyl- or 4- to 8-membered heterocycloalkyl-, which are unsubstituted or are mono- or disubstituted identically or differently by C₁-C₃-alkyl- or
   C₁-C₄-alkoxycarbonyl-, with the proviso that the 4- to 8-membered heterocycloalkyl- is not bonded via a nitrogen atom to the carbonyl or sulphonyl group in R¹.

Preference is given to compounds of the general formula (I) in which R⁷ is C₁-C₆-alkyl- which is unsubstituted or is monosubstituted by cyano, C₁-C₃-alkoxy-, C₁-C₃-alkylamino-, phenyl- or 4- to 8-membered heterocycloalkyl-,
where phenyl- for its part is unsubstituted or is mono-, di- or trisubstituted identically or differently by fluorine, chlorine, bromine, cyano, C₁-C₃-alkyl-, C₁-C₃-alkoxy-, and
where the 4- to 8-membered heterocycloalkyl- for its part is unsubstituted or mono- or disubstituted identically or differently by C₁-C₃-alkyl-.

Preference is given to compounds of the general formula (I) in which R⁷ is fluoro-C₁-C₃-alkyl.

Preference is given to compounds of the general formula (I) in which R⁷ is C₃-C₄-alkenyl- or C₃-C₄-alkynyl-.

Preference is given to compounds of the general formula (I) in which R⁷ is C₃-C₈-cycloalkyl- or 4- to 8-membered heterocycloalkyl- which are unsubstituted or are mono- or disubstituted identically or differently by C₁-C₃-alkyl- or C₁-C₄-alkoxycarbonyl-, with the proviso that the 4- to 8-membered heterocycloalkyl- is not bonded via a nitrogen atom to the carbonyl or sulphonyl group in R¹.

Particular preference is given to compounds of the general formula (I) in which R⁷ is C₁-C₄-alkyl-which is unsubstituted or is monosubstituted by cyano, phenyl- or 5- to 6-membered heterocycloalkyl-,
where phenyl- for its part is unsubstituted or is mono- or disubstituted identically or differently by fluorine, chlorine, cyano, methyl-,
methoxy-, and
where the 5- to 6-membered heterocycloalkyl- for its part is unsubstituted or is monosubstituted by C₁-C₃-alkyl-,
   or
   is fluoro-C₁-C₂-alkyl-,
   or
   is C₃-C₄-alkenyl-,
   or
   is C₃-C₆-cycloalkyl- or 5- to 6-membered heterocycloalkyl-, with the proviso that the 5- to 6-membered heterocycloalkyl- is not bonded via a nitrogen atom to the carbonyl or sulphonyl group in R¹.

Particular preference is given to compounds of the general formula (I) in which R¹ is C₁-C₄-alkyl-which is unsubstituted or is monosubstituted by cyano, phenyl- or 5- to 6-membered heterocycloalkyl-,
where phenyl- for its part is unsubstituted or is mono- or disubstituted identically or differently by fluorine, chlorine, cyano, methyl-,
methoxy-, and
where the 5- to 6-membered heterocycloalkyl- for its part is unsubstituted or is monosubstituted by C₁-C₃-alkyl-,
   or
   is C₃-C₈-cycloalkyl.

Particular preference is given to compounds of the general formula (I) in which R⁷ is C₁-C₄-alkyl-which is unsubstituted or is monosubstituted by cyano, phenyl- or 5- to 6-membered heterocycloalkyl-,
where phenyl- for its part is unsubstituted or is mono- or disubstituted identically or differently by fluorine, chlorine, cyano, methyl-, methoxy-, and
where the 5- to 6-membered heterocycloalkyl- for its part is unsubstituted or is monosubstituted by C₁-C₃-alkyl-.

Particular preference is given to compounds of the general formula (I) in which R⁷ is fluoro-C₁-C₂-alkyl-.

Particular preference is given to compounds of the general formula (I) in which R⁷ is C₃-C₄-alkenyl-.

Particular preference is given to compounds of the general formula (I) in which R⁷ is C₃-C₆-cycloalkyl- or 5- to 6-membered heterocycloalkyl-, with the proviso that the

5- to 6-membered heterocycloalkyl- is not bonded via a nitrogen atom to the carbonyl or sulphonyl group in R¹.

Particular preference is given to compounds of the general formula (I) in which R⁷ is C₃-C₈-cycloalkyl-.

Particular preference is given to compounds of the general formula (I) in which R⁷ is C₃-C₆-cycloalkyl-.

Particular preference is given to compounds of the general formula (I) in which R⁷ is 5- to 6-membered heterocycloalkyl-, with the proviso that the 5- to 6-membered heterocycloalkyl- is not bonded via a nitrogen atom to the carbonyl or sulphonyl group in R¹.

Very particular preference is given to compounds of the general formula (I) in which R⁷ is C₁-C₃-alkyl-, trifluoromethyl-, allyl-, C₃-C₄-cycloalkyl- or is tetrahydropyranyl-.

Very particular preference is given to compounds of the general formula (I) in which R⁷ is C₁-C₃-alkyl- or is cyclopropyl-.

Very particular preference is given to compounds of the general formula (I) in which R⁷ C₁-C₃-alkyl.

Very particular preference is given to compounds of the general formula (I) in which R⁷ is cyclopropyl-.

Exceptional preference is given to compounds of the general formula (I) in which R⁷ is methyl-, ethyl-, isopropyl-, trifluoromethyl-, allyl-, cyclopropyl-, cyclobutyl- or is tetrahydropyran-4-yl-.

Exceptional preference is given to compounds of the general formula (I) in which R⁷ is methyl-, isopropyl- or cyclopropyl-.

Exceptional preference is given to compounds of the general formula (I) in which R⁷ is methyl-.

Exceptional preference is given to compounds of the general formula (I) in which R⁷ is isopropyl-.

Exceptional preference is further given to compounds of the general formula (I) in which R⁷ is methyl-, ethyl-, isopropyl- or trifluoromethyl-.

Exceptional preference is given to compounds of the general formula (I) in which R⁷ is cyclopropyl-, cyclobutyl or tetrahydropyran-4-yl-.

Preference is given to compounds of the general formula (I) in which R⁸ is hydrogen, cyano, C₁-C₄-alkyl-, C₃-C₆-cycloalkyl- or —C(═O)OR².

Preference is given to compounds of the general formula (I) in which R⁸ is cyano, C₁-C₄-alkyl, C₃-C₆-cycloalkyl or —C(═O)OR¹².

Preference is given to compounds of the general formula (I) in which R⁸ is hydrogen, cyano or —C(═O)OR¹².

Preference is given to compounds of the general formula (I) in which R⁸ is cyano or —C(═O)OR¹².

Preference is given to compounds of the general formula (I) in which R⁸ is C₁-C₄-alkyl.

Preference is given to compounds of the general formula (I) in which R⁸ is cyano.

Preference is given to compounds of the general formula (I) in which R⁸ is —C(═O)OR¹².

Particular preference is given to compounds of the general formula (I) in which R⁸ is hydrogen, cyano, C₁-C₃-alkyl- or C₁-C₃-alkoxycarbonyl-.

Particular preference is given to compounds of the general formula (I) in which R⁸ is cyano, C₁-C₃-alkyl- or C₁-C₃-alkoxycarbonyl-.

Particular preference is given to compounds of the general formula (I) in which R⁸ is hydrogen or C₁-C₃-alkoxycarbonyl-.

Particular preference is given to compounds of the general formula (I) in which R⁸ is C₁-C₃-alkoxycarbonyl-.

Particular preference is given to compounds of the general formula (I) in which R⁸ is hydrogen or ethoxycarbonyl-.

Particular preference is given to compounds of the general formula (I) in which R⁸ is hydrogen.

Particular preference is given to compounds of the general formula (I) in which R⁸ is C₁-C₃-alkyl-.

Exceptional preference is given to compounds of the general formula (I) in which R⁸ is ethoxycarbonyl-.

Preference is given to compounds of the general formula (I) in which R⁹ is C₁-C₄-alkyl-.

Particular preference is given to compounds of the general formula (I) in which R⁹ is C₁-C₃-alkyl-.

Particular preference is given to compounds of the general formula (I) in which R⁹ is C₁-C₂-alkyl-.

Exceptional preference is given to compounds of the general formula (I) in which R⁹ is methyl-.

Preference is given to compounds of the general formula (I) in which R¹⁰ and R¹¹ are each independently hydrogen or are unsubstituted C₁-C₃-alkyl- or C₁-C₃-alkyl-monosubstituted by hydroxyl or oxo or are 5- to 6-membered heterocycloalkyl-,
where the 5- to 6-membered heterocycloalkyl- for its part is unsubstituted or mono- or disubstituted identically or differently by C₁-C₃-alkyl-,
   or in which

R¹⁰ and R¹¹ together with the nitrogen atom to which they are bonded are

4- to 7-membered heterocycloalkyl-, which is unsubstituted or is mono- or disubstitued identically or differently by hydroxyl, fluorine, oxo, C₁-C₃-alkyl-, fluoro-C₁-C₃-alkyl-, cyclopropyl-, cyclopropylmethyl-, acetyl- or tert-butoxycarbonyl-.

Preference is given to compounds of the general formula (I) in which R¹⁰ and R¹¹ are each independently hydrogen or are unsubstituted C₁-C₃-alkyl- or C₁-C₃-alkyl-monosubstituted by hydroxyl or oxo or are 5- to 6-membered heterocycloalkyl-,
where the 5- to 6-membered heterocycloalkyl- for its part is unsubstituted or mono- or disubstituted identically or differently by C₁-C₃-alkyl-.

Preference is given to compounds of the general formula (I) in which R¹⁰ and R¹¹ together with the nitrogen atom to which they are attached are 4- to 7-membered heterocycloalkyl which is unsubstituted or is mono- or disubstituted identically or differently by hydroxyl, fluorine, oxo, C₁-C₃-alkyl-, fluoro-C₁-C₃-alkyl-, cyclopropyl-, cyclopropylmethyl-, acetyl- or tert-butoxycarbonyl-.

Particular preference is given to compounds of the general formula (I) in which R¹⁰ and R¹¹ are each independently hydrogen or C₁-C₃-alkyl-,

or in which

R¹⁰ and R¹¹ together with the nitrogen atom to which they are bonded are

4- to 7-membered heterocycloalkyl-, which is unsubstituted or is mono- or disubstitued identically or differently by C₁-C₃-alkyl-, fluoro-C₁-C₃-alkyl- or tert-butoxycarbonyl-.

Particular preference is given to compounds of the general formula (I) in which R¹⁰ and R¹¹ are each independently hydrogen or C₁-C₃-alkyl-.

Particular preference is given to compounds of the general formula (I) in which R¹⁰ and R¹¹ together with the nitrogen atom to which they are attached are 4- to 7-membered heterocycloalkyl- which is unsubstituted or is mono- or disubstituted identically or differently by C₁-C₃-alkyl-, fluoro-C₁-C₃-alkyl-, or tert-butoxycarbonyl-.

Very particular preference is given to compounds of the general formula (I) in which R¹⁰ and R¹¹ together with the nitrogen atom to which they are attached are 5- to 6-membered heterocycloalkyl-which is unsubstituted or is monosubstituted by C₁-C₃-alkyl-.

Very particular preference is given to compounds of the general formula (I) in which R¹⁰ and R¹¹ together with the nitrogen atom to which they are attached are piperidinyl-, piperazinyl- or morpholinyl- which is unsubstituted or is monosubstituted by methyl-.

Exceptional preference is given to compounds of the general formula (I) in which R¹⁰ and R¹¹ together with the nitrogen atom to which they are attached are N-methylpiperazinyl-.

Preference is given to compounds of the general formula (I) in which R¹² is C₁-C₄-alkyl or benzyl-.

Preference is given to compounds of the general formula (I) in which R¹² is C₁-C₄-alkyl.

Preference is given to compounds of the general formula (I) in which R¹² is benzyl-.

Preference is given to compounds of the general formula (I) in which R¹² is methyl-.

Particular preference is given to compounds of the general formula (I) in which R¹² is C₁-C₃-alkyl-.

Particular preference is given to compounds of the general formula (I) in which R¹² is ethyl-.

Particular preference is given to compounds of the general formula (I) in which A is —NH—, X is —CH—, Y is —CH—, n is 0 or 1, R² is hydrogen, fluorine, chlorine, methyl-, methoxy-, trifluoromethoxy- or phenoxy-, where the phenyl present in phenoxy- is unsubstituted or is monosubstituted by fluorine or chlorine and in which R³ is methyl-, R⁴ is methyl- and R⁵ is hydrogen.

Particular preference is given to compounds of the general formula (I) in which A is —NH—, X is —CH—, Y is —CH—, n is 0 or 1, R² is hydrogen, methyl-, methoxy-, trifluoromethoxy-, phenoxy- or para-fluorophenoxy-, R³ is methyl-, R⁴ is methyl- and R⁵ is hydrogen.

Particular preference is given to compounds of the general formula (I) in which A is —NH—, X is —CH—, Y is —CH—, n is 0 or 1, R² is hydrogen, methyl- or methoxy-, R³ is methyl-, R⁴ is methyl- and R⁵ is hydrogen.

The specific radical definitions given in the particular combinations or preferred combinations of radicals are, irrespective of the particular combinations of radicals specified, also replaced as desired by radical definitions of other combination.

Very particular preference is given to combinations of two or more of the abovementioned preferred ranges.

Very particular preference is given to the following compounds of the general formula (I):
(3R)-1,3-Dimethyl-6-{[3-(methylsulphonyl)phenyl]amino}-4-(tetrahydro-2H-pyran-4-yl)-3,4-dihydropyrido[2,3-b]pyrazin-2(1H)-one;
(3R)-1,3-Dimethyl-6-{[2-methyl-5-(methylsulphonyl)phenyl]amino}-4-(tetrahydro-2H-pyran-4-yl)-3,4-dihydropyrido[2,3-b]pyrazin-2(1H)-one;
(3R)-6-{[2-Methoxy-5-(methylsulphonyl)phenyl]amino}-1,3-dimethyl-4-(tetrahydro-2H-pyran-4-yl)-3,4-dihydropyrido[2,3-b]pyrazin-2(1H)-one;
(3R)-6-[(1,1-dioxido-2,3-dihydro-1-benzothiophen-6-yl)amino]-1,3-dimethyl-4-(tetrahydro-2H-pyran-4-yl)-3,4-dihydropyrido[2,3-b]pyrazin-2(1H)-one;
(3R)-4-Cycloheptyl-1,3-dimethyl-6-{[3-(methylsulphonyl)phenyl]amino}-3,4-dihydropyrido[2,3-b]pyrazin-2(1H)-one;
(3R)-6-{[3-(Cyclopropylsulphonyl)phenyl]amino}-1,3-dimethyl-4-(tetrahydro-2H-pyran-4-yl)-3,4-dihydropyrido[2,3-b]pyrazin-2(1H)-one;
(3R)-6-{[3-(Isopropylsulphonyl)phenyl]amino}-1,3-dimethyl-4-(tetrahydro-2H-pyran-4-yl)-3,4-dihydropyrido[2,3-b]pyrazin-2(1H)-one;
(3R)-6-{[5-(Cyclopropylsulphonyl)-2-methoxyphenyl]amino}-1,3-dimethyl-4-(tetrahydro-2H-pyran-4-yl)-3,4-dihydropyrido[2,3-b]pyrazin-2(1H)-one;
(3R)-6-{[5-(Isopropylsulphonyl)-2-methoxyphenyl]amino}-1,3-dimethyl-4-(tetrahydro-2H-pyran-4-yl)-3,4-dihydropyrido[2,3-b]pyrazin-2(1H)-one;
Ethyl [(3-{[(3R)-1,3-dimethyl-2-oxo-4-(tetrahydro-2H-pyran-4-yl)-1,2,3,4-tetrahydropyrido[2,3-b]pyrazin-6-yl]amino}phenyl)(methyl)oxido-λ⁶-sulphanylidene]carbamate;
(3R)-1,3-Dimethyl-4-(1-methylpiperidin-4-yl)-6-{[3-(methylsulphonyl)phenyl]amino}-3,4-dihydropyrido[2,3-b]pyrazin-2(1H)-one;
(3R)-6-{[5-(Cyclopropylsulphonyl)-2-methoxyphenyl]amino}-1,3-dimethyl-4-(1-methylpiperidin-4-yl)-3,4-dihydropyrido[2,3-b]pyrazin-2(1H)-one;
(3R)-6-{[5-(Isopropylsulphonyl)-2-methoxyphenyl]amino}-1,3-dimethyl-4-(1-methylpiperidin-4-yl)-3,4-dihydropyrido[2,3-b]pyrazin-2(1H)-one;
(3R)-4-Isopropyl-6-{[2-methoxy-5-(methylsulphonyl)phenyl]amino}-1,3-dimethyl-3,4-dihydropyrido[2,3-b]pyrazin-2(1H)-one;
(3R)-6-{[5-(Cyclopropylsulphonyl)-2-methoxyphenyl]amino}-4-isopropyl-1,3-dimethyl-3,4-dihydropyrido[2,3-b]pyrazin-2(1H)-one;
(3R)-4-Isopropyl-6-{[5-(isopropylsulphonyl)-2-methoxyphenyl]amino}-1,3-dimethyl-3,4-dihydropyrido[2,3-b]pyrazin-2(1H)-one;
1,3-Dimethyl-6-{[3-(methylsulphonyl)phenyl]amino}-4-phenyl-3,4-dihydropyrido[2,3-b]pyrazin-2(1H)-one;
3′-{[(3R)-1,3-Dimethyl-2-oxo-4-(tetrahydro-2H-pyran-4-yl)-1,2,3,4-tetrahydropyrido[2,3-b]pyrazin-6-yl]amino}biphenyl-4-carbonitrile;
(3R)-6-{[3-(Isopropylsulphonyl)phenyl]amino}-1,3-dimethyl-4-(1-methylpiperidin-4-yl)-3,4-dihydropyrido[2,3-b]pyrazin-2(1H)-one;
(3R)-6-{[5-(Isopropylsulphonyl)-2-(trifluoromethoxy)phenyl]amino}-1,3-dimethyl-4-(1-methylpiperidin-4-yl)-3,4-dihydropyrido[2,3-b]pyrazin-2(1H)-one;
(3R)-6-({2-Methoxy-5-[(trifluoromethyl)sulphonyl]phenyl}amino)-1,3-dimethyl-4-(1-methylpiperidin-4-yl)-3,4-dihydropyrido[2,3-b]pyrazin-2(1H)-one;
(3R)-6-{[2-Methoxy-5-(tetrahydro-2H-pyran-4-ylsulphonyl)phenyl]amino}-1,3-dimethyl-4-(1-methylpiperidin-4-yl)-3,4-dihydropyrido[2,3-b]pyrazin-2(1H)-one;
(3R)-6-{[5-(Allylsulphonyl)-2-methoxyphenyl]amino}-1,3-dimethyl-4-(1-methylpiperidin-4-yl)-3,4-dihydropyrido[2,3-b]pyrazin-2(1H)-one;
tert-Butyl 4-[(3R)-6-{[5-(isopropylsulphonyl)-2-methoxyphenyl]amino}-1,3-dimethyl-2-oxo-2,3-dihydropyrido[2,3-b]pyrazin-4(1H)-yl]piperidine-1-carboxylate;
tert-Butyl 4-[(3R)-6-{[2-methoxy-5-(methylsulphonyl)phenyl]amino}-1,3-dimethyl-2-oxo-2,3-dihydropyrido[2,3-b]pyrazin-4(1H)-yl]piperidine-1-carboxylate;
(3R)-6-{[5-(Isopropylsulphonyl)-2-methoxyphenyl]amino}-1,3-dimethyl-4-(piperidin-4-yl)-3,4-dihydropyrido[2,3-b]pyrazin-2(1H)-one;
(3R)-6-{[2-Methoxy-5-(methylsulphonyl)phenyl]amino}-1,3-dimethyl-4-(piperidin-4-yl)-3,4-dihydropyrido[2,3-b]pyrazin-2(1H)-one;
(3R)-6-{[5-(Isopropylsulphonyl)-2-methoxyphenyl]amino}-1,3-dimethyl-4-[1-(2,2,2-trifluoroethyl)piperidin-4-yl]-3,4-dihydropyrido[2,3-b]pyrazin-2(1H)-one;
(3R)-4-[1-(2,2-Difluoroethyl)piperidin-4-yl]-6-{[5-(isopropylsulphonyl)-2-methoxyphenyl]amino}-1,3-dimethyl-3,4-dihydropyrido[2,3-b]pyrazin-2(1H)-one;
(3R)-6-{[3-(Isopropylsulphonyl)phenyl]amino}-1,3-dimethyl-4-[1-(3,3,3-trifluoropropyl)piperidin-4-yl]-3,4-dihydropyrido[2,3-b]pyrazin-2(1H)-one;
(3R)-6-{[5-(Isopropylsulphonyl)-2-methoxyphenyl]amino}-1,3-dimethyl-4-[1-(3,3,3-trifluoropropyl)piperidin-4-yl]-3,4-dihydropyrido[2,3-b]pyrazin-2(1H)-one;
(3R)-6-{[5-(Ethylsulphonyl)-2-(4-fluorophenoxy)phenyl]amino}-1,3-dimethyl-4-(1-methylpiperidin-4-yl)-3,4-dihydropyrido[2,3-b]pyrazin-2(1H)-one;
(3R)-6-{[5-(Ethylsulphonyl)-2-phenoxyphenyl]amino}-1,3-dimethyl-4-(1-methylpiperidin-4-yl)-3,4-dihydropyrido[2,3-b]pyrazin-2(1H)-one;
(3R)-6-{[2-Methoxy-5-(tetrahydro-2H-pyran-4-ylsulphonyl)phenyl]amino}-1,3-dimethyl-4-(tetrahydro-2H-pyran-4-yl)-3,4-dihydropyrido[2,3-b]pyrazin-2(1H)-one;
(3R)-6-{[5-(Cyclobutylsulphonyl)-2-methoxyphenyl]amino}-1,3-dimethyl-4-(tetrahydro-2H-pyran-4-yl)-3,4-dihydropyrido[2,3-b]pyrazin-2(1H)-one;
(3R)-6-{[5-(Ethylsulphonyl)-2-phenoxyphenyl]amino}-1,3-dimethyl-4-(tetrahydro-2H-pyran-4-yl)-3,4-dihydropyrido[2,3-b]pyrazin-2(1H)-one;
(3R)-6-{[5-(Ethylsulphonyl)-2-(4-fluorophenoxy)phenyl]amino}-1,3-dimethyl-4-(tetrahydro-2H-pyran-4-yl)-3,4-dihydropyrido[2,3-b]pyrazin-2(1H)-one;
(3R)-6-{[5-(Isopropylsulphonyl)-2-(trifluoromethoxy)phenyl]amino}-1,3-dimethyl-4-(tetrahydro-2H-pyran-4-yl)-3,4-dihydropyrido[2,3-b]pyrazin-2(1H)-one;
(3R)-6-({2-Methoxy-5-[(trifluoromethyl)sulphonyl]phenyl}amino)-1,3-dimethyl-4-(tetrahydro-2H-pyran-4-yl)-3,4-dihydropyrido[2,3-b]pyrazin-2(1H)-one;
(3R)-6-{[3-Methoxy-5-(methylsulphonyl)phenyl]amino}-1,3-dimethyl-4-(tetrahydro-2H-pyran-4-yl)-3,4-dihydropyrido[2,3-b]pyrazin-2(1H)-one;
(3R)-1,3-Dimethyl-6-{[3-(4-methylpiperazin-1-yl)phenyl]amino}-4-(tetrahydro-2H-pyran-4-yl)-3,4-dihydropyrido[2,3-b]pyrazin-2(1H)-one;
(3R)-1,3-Dimethyl-6-[(2-methylpyridin-4-yl)amino]-4-(tetrahydro-2H-pyran-4-yl)-3,4-dihydropyrido[2,3-b]pyrazin-2(1H)-one;
(3R)-1,3-Dimethyl-4-(tetrahydro-2H-pyran-4-yl)-6-{[3-(trifluoromethyl)phenyl]amino}-3,4-dihydropyrido[2,3-b]pyrazin-2(1H)-one and
(3R)-1,3-Dimethyl-6-{[3-(S-methylsulphonimidoyl) phenyl]amino}-4-(tetrahydro-2H-pyran-4-yl)-3,4-dihydropyrido[2,3-b]pyrazin-2(1H)-one,
   and diastereomers, racemates, polymorphs and physiologically acceptable salts thereof.

### DEFINITIONS

C₁-C₆-Alkyl or a C₁-C₆-alkyl group is understood to mean a straight-chain or branched, saturated monovalent hydrocarbon radical such as a methyl, ethyl, propyl, butyl, pentyl, hexyl, isopropyl, isobutyl, sec-butyl, tert-butyl, isopentyl, 2-methylbutyl, 1-methylbutyl, 1-ethylpropyl, 1,2-dimethylpropyl, neopentyl, 1,1-dimethylpropyl,

4-methylpentyl, 3-methylpentyl, 2-methylpentyl, 1-methylpentyl, 2-ethylbutyl, 1-ethylbutyl, 3,3-dimethylbutyl, 2,2-dimethylbutyl, 1,1-dimethylbutyl, 2,3-dimethylbutyl,
1,3-dimethylbutyl or 1,2-dimethylbutyl radical.

Preferably, C₁-C₆-alkyl, or a C₁-C₆-alkyl group, is understood to mean C₁-C₄-alkyl, C₂-C₄-alkyl or C₂-C₅-alkyl, particularly preferably C₁-C₃-alkyl or a methyl, ethyl, propyl or isopropyl radical.

C₂-C₅-Alkylene, or a C₂-C₅-alkylene group, is understood to mean a straight-chain or branched, saturated, bivalent hydrocarbon radical, for example an ethylene, propylene, butylene, pentylene, isopropylene, isobutylene, sec-butylene, tert-butylene, isopentylene, 2-methylbutylene, 1-methylbutylene, 1-ethylpropylene, 1,2-dimethylpropylene, neopentylene or 1,1-dimethylpropylene radical.

C₂-C₄-Alkenyl-, or a C₂-C₄-alkenyl group, is understood to mean a straight-chain or branched, monovalent hydrocarbon radical having one or two C═C double bonds, for example an ethenyl, (E)-prop-2-enyl, (Z)-prop-2-enyl, allyl (prop-1-enyl), allenyl, buten-1-yl or buta-1,3-dienyl radical. Preference is given to ethenyl- and allyl-.

C₂-C₄-Alkynyl, or a C₂-C₄-alkynyl group, is understood to mean a straight-chain or branched, monovalent hydrocarbon radical having one C≡C triple bond, for example an ethynyl, propargyl (prop-1-ynyl) or butyn-1-yl radical. Preference is given to ethynyl and propargyl.

C₁-C₄-Alkoxy-, or a C₁-C₄-alkoxy group, is understood to mean a straight-chain or branched, saturated alkyl ether radical —O-alkyl, for example a methoxy, ethoxy, n-propoxy, isopropoxy or tert-butoxy radical.

Preferably, C₁-C₄-alkoxy, or a C₁-C₄-alkoxy group, is understood to mean C₁-C₃-alkoxy-, particularly preferably a methoxy or ethoxy radical.

C₁-C₄-Alkylthio-, or a C₁-C₄-alkylthio group, is understood to mean a straight-chain or branched, saturated alkyl thioether radical —S-alkyl, for example a methylthio, ethylthio, n-propylthio, isopropylthio or tert-butylthio radical.

Preferably, C₁-C₄-alkylthio-, or a C₁-C₄-alkylthio group, is understood to mean C₁-C₃-alkylthio-, more preferably a methylthio and ethylthio radical.

C₁-C₃-Alkylamino-, or a C₁-C₃-alkylamino group, is understood to mean an amino radical having one or two (selected independently of each other) alkyl substituents having 1 to 3 carbon atoms as defined above.

(C₁-C₃)-Alkylamino is, for example, a monoalkylamino radical having 1 to 3 carbon atoms or a dialkylamino radical having 1 to 3 carbon atoms each per alkyl substituent.

Examples include:

methylamino-, ethylamino-, n-propylamino-, isopropylamino-, N,N-dimethylamino-, N,N-diethylamino-, N-ethyl-N-methylamino-, N-methyl-N-n-propylamino- and N-isopropyl-N-n-propylamino-.

A heteroatom is understood to mean —O—, NH—, ═N— or —S—, including the oxidized forms thereof —S(═O)— and —S(═O)₂— and a sulphoximine —S(═O)(═NH)— derived from —S(═O)₂—. The heteroatom —NH— may optionally be substituted by C₁-C₃-alkyl, C₁-C₃-alkylcarbonyl, C₁-C₄-alkoxycarbonyl, or —S(═O)₂—C₁-C₃-alkyl. The ═NH of the abovementioned sulphoximine may optionally be substituted by C₁-C₃-alkyl, C₁-C₃-alkylcarbonyl-,

C₁-C₄-alkoxycarbonyl-.

Preference is given to an oxygen and a nitrogen atom.

Oxo, or an oxo substituent, is understood to mean a double-bonded oxygen atom ═O. Oxo may be bonded to atoms of suitable valency, for example to a saturated carbon atom or to sulphur.

Preference is given to the bond to carbon to form a carbonyl group.

Preference is further given to the bond of two double-bonded oxygen atoms to sulphur, forming a sulphonyl group —(S═O)₂—.

Halogen is understood to mean fluorine, chlorine, bromine or iodine.

Fluorine, chlorine bromine or iodine which is an optional substituent on the phenyl ring may be in the ortho, meta or para position. Preference is given to fluorine and chlorine.

The preferred position is the meta and para position.

A halo-C₁-C₄-alkyl radical is understood to mean a C₁-C₄-alkyl radical having at least one halogen substituent, preferably having at least one fluorine substituent.

Preference is given to fluoro-C₁-C₃-alkyl radicals, for example difluoromethyl-, trifluoromethyl-, 2,2,2-trifluoroethyl- and pentafluoroethyl-.

Particular preference is given to perfluorinated alkyl radicals such as trifluoromethyl- and pentafluoroethyl-.

Phenyl-C₁-C₃-alkyl is understood to mean a group composed of an optionally substituted phenyl radical and a C₁-C₃-alkyl group, and bonded to the rest of the molecule via the C₁-C₃-alkyl group.

Preference is given to benzyl.

A halo-C₁-C₄-alkoxy radical is understood to mean a C₁-C₄-alkoxy radical having at least one halogen substituent, preferably having at least one fluorine substituent.

Preference is given to fluoro-C₁-C₃-alkoxy radicals, for example difluoromethoxy, trifluoromethoxy or 2,2,2-trifluoroethoxy radicals.

A halo-C₁-C₄-alkylthio radical is understood to mean a C₁-C₄-alkylthio radical having at least one halogen substituent, preferably having at least one fluorine substituent.

Preference is given to fluoro-C₁-C₃-alkylthio radicals, especially trifluoromethylthio-.

A C₁-C₃-alkylcarbonyl radical is understood to mean a C₁-C₃-alkyl-C(═O) group. Preference is given to acetyl- and propanoyl-.

A C₁-C₄-alkoxycarbonyl radical is understood to mean a C₁-C₄-alkoxy-C(═O)— group. Preference is given to methoxycarbonyl-, ethoxycarbonyl- and tert-butoxycarbonyl-.

A C₁-C₄-alkoxy-C₁-C₄-alkyl radical is understood to mean a C₁-C₄-alkoxy-substituted C₁-C₄-alkyl radical such as, for example, methoxymethyl, methoxyethyl, ethoxymethyl and ethoxyethyl.

Aryl is understood to mean an unsaturated, fully conjugated system which is formed from carbon atoms and has 3, 5 or 7 conjugated double bonds, for example phenyl, naphthyl or phenanthryl.

Preference is given to phenyl.

Heteroaryl- is understood to mean ring systems which have an aromatically conjugated ring system and contain at least one and up to five heteroatoms as defined above.

These ring systems may have 5, 6 or 7 ring atoms, or else, in the case of fused or benzofused ring systems, combinations of 5- and 6-membered ring systems, 5- and 5-membered ring systems, or else 6- and 6-membered ring systems. Examples which may be mentioned are ring systems such as pyrrolyl, pyrazolyl, imidazolyl, triazolyl, tetrazolyl, furyl, thienyl, oxazolyl, thiazolyl, isoxazolyl, oxadiazolyl, thiadiazolyl, pyridinyl, pyrimidinyl, pyrazinyl, triazinyl, oxazinyl, indolyl, benzimidazolyl, indazolyl, benzotriazolyl, benzothiazolyl, benzoxazolyl, benzofuryl, benzothienyl, quinolinyl, isoquinolinyl, cinnolinyl, quinazolinyl, quinoxalinyl, imidazopyridinyl or else benzoxazinyl.

Preference is given to 5- to 6-membered, monocyclic heteroaryl, for example pyrrolyl, pyrazolyl, imidazolyl, triazolyl, tetrazolyl, furyl, thienyl, oxazolyl, thiazolyl, isoxazolyl, oxadiazolyl, thiadiazolyl, pyridinyl, pyrimidinyl, pyrazinyl, triazinyl.

C₃-C₆-Cycloalkyl, C₃-C₈-cycloalkyl, and C₅-C₈-cycloalkyl are understood to mean a monocyclic, saturated ring system formed exclusively from carbon atoms and having, respectively, 3 to 6, 3 to 8, and 5 to 8 atoms. Examples are cyclopropyl, cyclobutyl, cyclopentyl, cyclohexyl, cycloheptyl or cyclooctyl.

C₄-C₆-Cycloalkenyl, C₄-C₈-cycloalkenyl, and C₅-C₈-cycloalkenyl are understood to mean a monocyclic, mono- or polyunsaturated, non-aromatic ring system formed exclusively from carbon atoms and having, respectively, 4 to 6, 4 to 8, and 5 to 8 atoms. Examples are cyclobuten-1-yl, cyclopenten-1-yl, cyclohexen-2-yl, cyclohexen-1-yl and cycloocta-2,5-dienyl.

Heterocycloalkyl is understood to mean a 4- to 8-membered monocyclic, saturated ring system having 1 to 3 heteroatoms as defined above in any combination. Preference is given to 4- to 7-membered heterocycloalkyl groups, particular preference to 5- to 6-membered heterocycloalkyl groups.

Examples include pyrrolidinyl, piperidinyl, tetrahydrofuranyl, tetrahydropyranyl, oxetanyl, azetidinyl, azepanyl, morpholinyl, thiomorpholinyl and piperazinyl.

Heterocycloalkenyl is understood to mean a 4- to 8-membered monocyclic, mono- or polyunsaturated, non-aromatic ring system having 1 to 3 heteroatoms as defined above in any combination. Preference is given to 4- to 7-membered heterocycloalkyl groups, particular preference to 5- to 6-membered heterocycloalkyl groups. Examples include 4H-pyranyl, 2H-pyranyl, 2,5-dihydro-1H-pyrrolyl, [1,3]dioxolyl, 4H-[1,3,4]thiadiazinyl, 2,5-dihydrofuranyl, 2,3-dihydrofuranyl, 2,5-dihydrothiophenyl, 2,3-dihydrothiophenyl, 4,5-dihydrooxazolyl and 4H-[1,4]thiazinyl.

Compounds according to the invention are the compounds of the general formula (I) and the salts, solvates and solvates of the salts thereof, the compounds, encompassed by the general formula (I), of the formulae specified hereinafter and the salts, solvates and solvates of the salts thereof, and the compounds encompassed by the general formula (I) and specified hereinafter as working examples and the salts, solvates and solvates of the salts thereof, to the extent that the compounds encompassed by the general formula (I) and specified hereinafter are not already salts, solvates and solvates of the salts.

The present invention is likewise considered to encompass the use of the salts of the compounds according to the invention.

Preferred salts in the context of the present invention are physiologically acceptable salts of the compounds of the invention. However, the invention also encompasses salts which themselves are unsuitable for pharmaceutical applications but which can be used, for example, for the isolation or purification of the compounds according to the invention.

Physiologically acceptable salts of the compounds according to the invention include acid addition salts of mineral acids, carboxylic acids and sulphonic acids, e.g. salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, trifluoroacetic acid, propionic acid, lactic acid, tartaric acid, malic acid, citric acid, fumaric acid, maleic acid and benzoic acid.

The present invention further provides all the possible crystalline and polymorphous forms of the inventive compounds, where the polymorphs may be present either as single polymorphs or as a mixture of a plurality of polymorphs in all concentration ranges.

The present invention also relates to medicaments comprising the compounds according to the invention together with at least one or more further active compounds, especially for prophylaxis and/or treatment of neoplastic disorders.

Solvates in the context of the invention are described as those forms of the compounds of the invention which form a complex in the solid or liquid state by coordination with solvent molecules. Hydrates are a specific form of the solvates in which the coordination is with water. Solvates preferred in the context of the present invention are hydrates.

The compounds according to the invention may, depending on their structure, exist in different stereoisomeric forms, i.e. in the form of configurational isomers or else optionally as conformational isomers. The compounds according to the invention may have a centre of asymmetry at the carbon atom to which R⁴ and R⁵ are attached (C-3). They may therefore take the form of pure enantiomers, racemates, or else of diastereomers or mixtures thereof when one or more of the substituents described in the formula (I) contains a further element of asymmetry, for example a chiral carbon atom. The present invention therefore also encompasses diastereomers and the respective mixtures thereof. The pure stereoisomers can be isolated from such mixtures in a known manner; chromatography processes are preferably used for this, in particular HPLC chromatography on a chiral or achiral phase.

In general, the enantiomers according to the invention inhibit the target proteins to different degrees and have different activity in the cancer cell lines studied. The more active enantiomer is preferred, which is often that in which the centre of asymmetry represented by the carbon atom bonded to R⁴ and R⁵ has (R) configuration.

The present invention further provides enantiomer mixtures of the (3R)-configured compounds according to the invention with their (3S) enantiomers, especially the corresponding racemates and enantiomer mixtures in which the (3R) form predominates.

If the compounds of the invention can occur in tautomeric forms, the present invention encompasses all the tautomeric forms.

The present invention also encompasses all suitable isotopic variants of the compounds of the invention. An isotopic variant of a compound of the invention is understood here to mean a compound in which at least one atom within the compound of the invention has been exchanged for another atom of the same atomic number, but with a different atomic mass from the atomic mass which usually or predominantly occurs in nature. Examples of isotopes which can be incorporated into a compound according to the invention are those of hydrogen, carbon, nitrogen, oxygen, phosphorus, sulphur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²⁴I, ¹²⁹I and ¹³¹I. Particular isotopic variants of a compound of the invention, especially those in which one or more radioactive isotopes have been incorporated, may be beneficial, for example, for the examination of the mechanism of action or of the active compound distribution in the body; due to the comparatively easy preparability and detectability, especially compounds labelled with ³H or ¹⁴C isotopes are suitable for this purpose. In addition, the incorporation of isotopes, for example of deuterium, may lead to particular therapeutic benefits as a consequence of greater metabolic stability of the compound, for example an extension of the half-life in the body or a reduction in the active dose required; such modifications of the compounds according to the invention may therefore in some cases also constitute a preferred embodiment of the present invention. Isotopic variants of the compounds according to the invention can be prepared by the processes known to those skilled in the art, for example by the methods described further down and the procedures described in the working examples, by using corresponding isotopic modifications of the respective reagents and/or starting compounds.

The compounds of the invention can act systemically and/or locally. For this purpose, they can be administered in a suitable manner, for example by the oral, parenteral, pulmonary, nasal, sublingual, lingual, buccal, rectal, dermal, transdermal, conjunctival, otic route, or as an implant or stent.

The compounds according to the invention can be administered in administration forms suitable for these administration routes.

Suitable administration forms for oral administration are all administration forms capable of releasing the compounds according to the invention rapidly. Here, the compounds according to the invention can be present in crystalline, amorphous and/or dissolved form, for example in tablets (non-coated or coated tablets, for example coated with enteric, slowly dissolving or insoluble coats which control the release of the compound according to the invention), in tablets which disintegrate rapidly in the oral cavity, in films/wafers, in films/lyophylizates, in capsules (for example hard gelatin capsules or soft gelatin capsules), in sugar-coated tablets, in granules, in pellets, in powders, in emulsions, in suspensions, in aerosols or in solutions.

Parenteral administration can bypass an absorption step (for example intravenously, intraarterially, intracardially, intraspinally or intralumbarly) or include an absorption (for example intramuscularly, subcutaneously, intracutaneously, percutaneously or intraperitoneally). Administration forms suitable for parenteral administration include preparations for injection and infusion in the form of solutions, suspensions, emulsions, lyophilizates or sterile powders.

Suitable administration forms for the other administration routes are, for example, pharmaceutical forms for inhalation (including powder inhalers, nebulizers), nasal drops, solutions or sprays; tablets for lingual, sublingual or buccal administration, films/wafers or capsules, suppositories, preparations for the ears or eyes, vaginal capsules, aqueous suspensions (lotions, shaking mixtures), lipophilic suspensions, ointments, creams, transdermal therapeutic systems (for example patches), milk, pastes, foams, dusting powders, implants or stents.

The compounds of the invention can be converted to the administration forms mentioned. This can be accomplished in a manner known per se to the person skilled in the art, by mixing with inert nontoxic pharmaceutically suitable auxiliaries. These auxiliaries include carriers (for example microcrystalline cellulose, lactose, mannitol), solvents (e.g. liquid polyethylene glycols), emulsifiers and dispersing or wetting agents (for example sodium dodecylsulphate, polyoxysorbitan oleate), binders (for example polyvinylpyrrolidone), synthetic and natural polymers (for example albumin), stabilizers (e.g. antioxidants, for example ascorbic acid), colourants (e.g. inorganic pigments, for example iron oxides) and flavour and/or odour correctants.

The present invention furthermore provides medicaments which comprise the compounds according to the invention, typically together with one or more inert, nontoxic, pharmaceutically suitable excipients, and the use thereof for the aforementioned purposes.

The compounds according to the invention are formulated to give pharmaceutical preparations in a manner known per se to those skilled in the art, by converting the active compound(s) to the desired administration form with the excipients customary in the pharmaceutical formulation.

The auxiliaries used may, for example, be carrier substances, fillers, disintegrants, binders, humectants, glidants, absorbents and adsorbents, diluents, solvents, cosolvents, emulsifiers, solubilizers, taste correctants, colorants, preservatives, stabilizers, wetting agents, salts for modifying osmotic pressure or buffers. Reference should be made to Remington's Pharmaceutical Science, 15th ed. Mack Publishing Company, East Pa. (1980).

The pharmaceutical formulations may be in solid form, for example in the form of tablets, coated tablets, pills, suppositories, capsules, transdermal systems, or in semisolid form, for example in the form of ointments, creams, gels, suppositories, emulsions, or in liquid form, for example in the form of solutions, tinctures, suspensions or emulsions.

Auxiliaries in the context of the invention may, for example, be salts, saccharides (mono-, di-, tri-, oligo- and/or polysaccharides), proteins, amino acids, peptides, fats, waxes, oils, hydrocarbons and derivatives thereof, and the auxiliaries may be of natural origin or be obtained by synthetic or partially synthetic means.

Useful forms for oral or peroral administration are especially tablets, sugar-coated tablets, capsules, pills, powders, granules, pastilles, suspensions, emulsions or solutions.

Useful forms for parenteral administration are especially suspensions, emulsions, and particularly solutions.

The compounds according to the invention are suitable for prophylaxis and/or therapy of hyperproliferative disorders, for example psoriasis, keloids and other hyperplasias which affect the skin, and for prophylaxis and/or therapy of benign prostate hyperplasias (BPH), solid tumours and haematological tumours.

Solid tumours that can be treated in accordance with the invention are, for example, tumours of the breast, the respiratory tract, the brain, the reproductive organs, the gastrointestinal tract, the urogenital tract, the eye, the liver, the skin, the head and the neck, the thyroid gland, the parathyroid gland, the bones, and the connective tissue and metastases of these tumours.

Haematological tumours that can be treated are, for example, multiple myeloma, lymphoma or leukaemia.

Breast tumours that can be treated are, for example, mammary carcinoma with positive hormone receptor status, mammary carcinoma with negative hormone receptor status, Her-2-positive mammary carcinoma, hormone receptor- and Her-2-negative mammary carcinoma, BRCA-associated mammary carcinoma and inflammatory mammary carcinoma.

Tumours of the respiratory tract that can be treated are, for example, non-small-cell bronchial carcinoma and small-cell bronchial carcinoma.

Brain tumours that can be treated are, for example, glioma, glioblastoma, astrocytoma, meningioma and medulloblastoma.

Tumours of the male reproductive organs that can be treated are, for example, prostate carcinoma, malignant epididymal tumours, malignant testicular tumours and penile carcinoma.

Tumours of the female reproductive organs that can be treated are, for example, endometrial carcinoma, cervical carcinoma, ovarian carcinoma, vaginal carcinoma and vulvar carcinoma.

Tumours of the gastrointestinal tract that can be treated are, for example, colorectal carcinoma, anal carcinoma, gastric carcinoma, pancreatic carcinoma, oesophageal carcinoma, gallbladder carcinoma, small-intestinal carcinoma, salivary gland carcinoma, neuroendocrine tumours and gastrointestinal stromal tumours.

Tumours of the urogenital tract that can be treated are, for example, urinary bladder carcinoma, renal cell carcinoma, and carcinoma of the renal pelvis and of the urinary tract.

Tumours of the eye that can be treated are, for example, retinoblastoma and intraocular melanoma.

Tumours of the liver that can be treated are, for example, hepatocellular carcinoma and cholangiocellular carcinoma.

Tumours of the skin that can be treated are, for example, malignant melanoma, basalioma, spinalioma, Kaposi's sarcoma and Merkel cell carcinoma.

Tumours of the head and neck that can be treated are, for example, laryngeal carcinoma and carcinoma of the pharynx and of the oral cavity.

Sarcomas that can be treated are, for example, soft tissue sarcoma and osteosarcoma.

Lymphomas that can be treated are, for example, non-Hodgkin's lymphoma, Hodgkin's lymphoma, cutaneous lymphoma, lymphoma of the central nervous system and AIDS-associated lymphoma.

Leukaemias that can be treated are, for example, acute myeloid leukaemia, chronic myeloid leukaemia, acute lymphatic leukaemia, chronic lymphatic leukaemia and hair cell leukaemia.

Advantageously, the compounds according to the invention can be used for prophylaxis and/or treatment of leukaemia, especially acute myeloid leukaemia, prostate carcinoma, especially androgen receptor-positive prostate carcinoma, cervical carcinoma, mammary carcinoma, especially hormone receptor-negative, hormone receptor-positive or BRCA-associated mammary carcinoma, pancreatic carcinoma, renal cell carcinoma, hepatocellular carcinoma, melanoma and other skin tumours, non-small-cell bronchial carcinoma, endometrial carcinoma and colorectal carcinoma.

The compounds according to the invention may be used advantageously for prophylaxis and/or therapy of leukaemias, especially acute myeloid leukaemias, prostate carcinomas, especially androgen receptor-positive prostate carcinomas, mammary carcinomas, especially oestrogen receptor alpha-negative mammary carcinomas, melanomas or multiple myelomas.

The compounds according to the invention are also suitable for prophylaxis and/or treatment of benign hyperproliferative diseases, for example endometriosis, leiomyoma and benign prostate hyperplasia.

The compounds according to the invention are also suitable for prophylaxis and/or treatment of systemic inflammatory diseases, especially LPS-induced endotoxic shock and/or bacteria-induced sepsis.

The compounds according to the invention are also suitable for prophylaxis and/or treatment of inflammatory or autoimmune disorders, for example:
pulmonary disorders associated with inflammatory, allergic and/or proliferative processes: chronic obstructive pulmonary disorders of any origin, particularly bronchial asthma; bronchitis of different origin; all forms of restrictive pulmonary disorders, particularly allergic alveolitis; all forms of pulmonary oedema, particularly toxic pulmonary oedema; sarcoidoses and granulomatoses, particularly Boeck's disease,
rheumatic disorders/autoimmune disorders/joint disorders associated with inflammatory, allergic and/or proliferative processes: all forms of rheumatic disorders, especially rheumatoid arthritis, acute rheumatic fever, polymyalgia rheumatica; reactive arthritis; inflammatory soft-tissue disorders of other origin; arthritic symptoms in the case of degenerative joint disorders (arthroses); traumatic arthritis; collagenoses of any origin, for example systemic lupus erythematosus, sclerodermia, polymyositis, dermatomyositis, Sjögren's syndrome, Still's syndrome, Felty's syndrome,
allergies associated with inflammatory and/or proliferative processes: all forms of allergic reactions, for example angiooedema, hay fever, insect bites, allergic reactions to medicaments, blood derivatives, contrast agents, etc., anaphylactic shock, urticaria, contact dermatitis,
vascular inflammation (vasculitis): panarteritis nodosa, temporal arteritis, erythema nodosum,
dermatological disorders associated with inflammatory, allergic and/or proliferative processes: atopic dermatitis; psoriasis; pityriasis rubra pilaris; erythematous disorders triggered by different noxae, for example radiation, chemicals, burns, etc.; bullous dermatoses; lichenoid disorders; pruritus; seborrhoeic eczema; rosacea; pemphigus vulgaris; erythema exsudativum multiforme; balanitis; vulvitis; hair loss, such as alopecia areata; cutaneous T-cell lymphoma
renal disorders associated with inflammatory, allergic and/or proliferative processes: nephrotic syndrome; all nephritides,
hepatic disorders associated with inflammatory, allergic and/or proliferative processes: acute hepatic disintegration; acute hepatitis of different origin, for example viral, toxic, medicament-induced; chronic aggressive and/or chronic intermittent hepatitis,
gastrointestinal disorders associated with inflammatory, allergic and/or proliferative processes: regional enteritis (Crohn's disease); ulcerative colitis; gastritis; reflux oesophagitis; gastroenteritides of other origin, e.g. indigenous sprue,
proctological disorders associated with inflammatory, allergic and/or proliferative processes: anal eczema; fissures; haemorrhoids; idiopathic proctitis,
ocular disorders associated with inflammatory, allergic and/or proliferative processes: allergic keratitis, uveitis, iritis; conjunctivitis; blepharitis; optic neuritis; chlorioditis; sympathetic ophthalmia,
disorders of the ear-nose-throat region associated with inflammatory, allergic and/or proliferative processes: allergic rhinitis, hay fever; otitis externa, for example caused by contact eczema, infection, etc.; otitis media,
neurological disorders associated with inflammatory, allergic and/or proliferative processes: cerebral oedema, particularly tumour-related cerebral oedema; multiple sclerosis; acute encephalomyelitis; meningitis; various forms of seizure, for example West's syndrome,
haematological disorders associated with inflammatory, allergic and/or proliferative processes: congenital haemolytic anaemia; idiopathic thrombocytopenia,
neoplastic disorders associated with inflammatory, allergic and/or proliferative processes: acute lymphatic leukaemia; malignant lymphoma; lymphogranulomatoses; lymphosarcoma; extensive metastases, particularly in the case of mammary, bronchial and prostate carcinoma,
endocrine disorders associated with inflammatory, allergic and/or proliferative processes: endocrine orbitopathy; thyrotoxic crisis; de Quervain's thyroiditis; Hashimoto's thyroiditis; Basedow's disease,
organ and tissue transplants, graft-versus-host disease,
severe states of shock, for example anaphylactic shock, systemic inflammatory response syndrome (SIRS),
substitution therapy in the case of: congenital primary renal insufficiency, for example congenital adrenogenital syndrome; acquired primary renal insufficiency, for example Addison's disease, autoimmune adrenalitis, for example postinfectious, tumours, metastases, etc; congenital secondary renal insufficiency, for example congenital hypopituitarism; acquired secondary renal insufficiency, for example postinfectious, tumours, etc.,
emesis associated with inflammatory, allergic and/or proliferative processes, for example in combination with a 5-HT3 antagonist in the case of cytostatic-induced vomiting,
pain of inflammatory origin, for example lumbago.

The compounds according to the invention are also suitable for the treatment of viral disorders, for example infections caused by papilloma viruses, herpes viruses, Epstein-Barr viruses, hepatitis B or C viruses, and human immunodeficiency viruses.

The compounds according to the invention are also suitable for the treatment of atherosclerosis, dyslipidaemia, hypercholesterolaemia, hypertriglyceridaemia, peripheral vascular disorders, cardiovascular disorders, angina pectoris, ischaemia, stroke, myocardial infarction, angioplastic restenosis, hypertension, thrombosis, obesity, endotoxaemia.

The compounds according to the invention are also suitable for the treatment of neurodegenerative diseases, for example multiple sclerosis, Alzheimer's disease and Parkinson's disease.

These disorders are well characterized in man, but also exist in other mammals.

The present invention further provides for the use of the compounds according to the invention as a medicament, in particular for prophylaxis and/or therapy of neoplastic disorders.

The present invention further provides the use of the compounds according to the invention for prophylaxis and/or treatment of leukaemia, especially acute myeloid leukaemia, prostate carcinoma, especially androgen receptor-positive prostate carcinoma, cervical carcinoma, mammary carcinoma, especially hormone receptor-negative, hormone receptor-positive or BRCA-associated mammary carcinoma, pancreatic carcinoma, renal cell carcinoma, hepatocellular carcinoma, melanoma and other skin tumours, non-small-cell bronchial carcinoma, endometrial carcinoma and colorectal carcinoma.

The present invention further relates to the use of the compounds according to the invention for prophylaxis and/or therapy of leukaemias, especially acute myeloid leukaemias, prostate carcinomas, especially androgen receptor-positive prostate carcinomas, mammary carcinomas, especially oestrogen receptor alpha-negative mammary carcinomas, melanomas or multiple myelomas.

The invention furthermore provides for the use of the compounds according to the invention for production of a medicament.

The present invention furthermore provides for the use of the compounds according to the invention for production of a medicament for prophylaxis and/or treatment of neoplastic disorders.

The present application furthermore provides for the use of the compounds according to the invention for production of a medicament for prophylaxis and/or therapy of leukaemia, especially acute myeloid leukaemia, prostate carcinoma, especially androgen receptor-positive prostate carcinoma, cervical carcinoma, mammary carcinoma, especially hormone receptor-negative, hormone receptor-positive or BRCA-associated mammary carcinoma, pancreatic carcinoma, renal cell carcinoma, hepatocellular carcinoma, melanoma and other skin tumours, non-small-cell bronchial carcinoma, endometrial carcinoma and colorectal carcinoma.

The present invention furthermore provides for the use of the compounds according to the invention for production of a medicament for prophylaxis and/or therapy of leukaemias, especially acute myeloid leukaemias, prostate carcinomas, especially androgen receptor-positive prostate carcinomas, mammary carcinomas, especially oestrogen receptor alpha-negative mammary carcinomas, melanomas or multiple myelomas.

The present invention furthermore provides for the use of the compounds according to the invention for prophylaxis and/or treatment of neoplastic disorders.

The present invention furthermore provides for the use of the compounds according to the invention for prophylaxis and/or treatment of leukaemia, especially acute myeloid leukaemia, prostate carcinoma, especially androgen receptor-positive prostate carcinoma, cervical carcinoma, mammary carcinoma, especially hormone receptor-negative, hormone receptor-positive or BRCA-associated mammary carcinoma, pancreatic carcinoma, renal cell carcinoma, hepatocellular carcinoma, melanoma and other skin tumours, non-small-cell bronchial carcinoma, endometrial carcinoma and colorectal carcinoma.

The present invention further relates to the use of the compounds according to the invention for prophylaxis and/or therapy of leukaemias, especially acute myeloid leukaemias, prostate carcinomas, especially androgen receptor-positive prostate carcinomas, mammary carcinomas, especially oestrogen receptor alpha-negative mammary carcinomas, melanomas or multiple myelomas.

The present invention furthermore provides pharmaceutical formulations in the form of tablets comprising one of the compounds according to the invention for prophylaxis and/or treatment of leukaemia, especially acute myeloid leukaemia, prostate carcinoma, especially androgen receptor-positive prostate carcinoma, cervical carcinoma, mammary carcinoma, especially hormone receptor-negative, hormone receptor-positive or BRCA-associated mammary carcinoma, pancreatic carcinoma, renal cell carcinoma, hepatocellular carcinoma, melanoma and other skin tumours, non-small-cell bronchial carcinoma, endometrial carcinoma and colorectal carcinoma.

The present invention furthermore provides pharmaceutical formulations in the form of tablets comprising one of the compounds according to the invention for prophylaxis and/or therapy of leukaemias, especially acute myeloid leukaemias, prostate carcinomas, especially androgen receptor-positive prostate carcinomas, mammary carcinomas, especially oestrogen receptor alpha-negative mammary carcinomas, melanomas or multiple myelomas.

The invention furthermore provides for the use of the compounds according to the invention for treatment of disorders associated with proliferative processes.

The invention further provides for the use of the compounds according to the invention for treatment of benign hyperplasias, inflammation disorders, autoimmune disorders, sepsis, viral infections, vascular disorders and neurodegenerative disorders.

The compounds of the invention can be used alone or, if required, in combination with one or more further pharmacologically active substances, provided that this combination does not lead to undesirable and unacceptable side effects. The present invention therefore further provides medicaments comprising a compound according to the invention and one or more further active compounds, especially for prophylaxis and/or treatment of the aforementioned disorders.

For example, the compounds according to the invention can be combined with known antihyperproliferative, cytostatic or cytotoxic chemical and biological substances for treatment of cancer. The combination of the compounds according to the invention with other substances commonly used for cancer treatment, or else with radiotherapy, is particularly appropriate.

An illustrative but nonexhaustive list of active compounds suitable for combinations is as follows:

abiraterone acetate, abraxane, acolbifene, actimmune, actinomycin D (dactinomycin), afatinib, affinitak, afinitor, aldesleukin, alendronic acid, alfaferone, alitretinoin, allopurinol, aloprim, aloxi, alpharadin, altretamine, aminoglutethimide, aminopterin, amifostine, amrubicin, amsacrine, anastrozole, anzmet, apatinib, aranesp, arglabin, arsenic trioxide, aromasin, arzoxifene, asoprisnil, L-asparaginasee, atamestane, atrasentan, avastin, axitinib, 5-azacytidine, azathioprine, BCG or tice-BCG, bendamustine, bestatin, betamethasone acetate, betamethasone sodium phosphate, bexarotene, bicalutamide, bleomycin sulphate, broxuridine, bortezomib, bosutinib, busulphan, cabazitaxel, calcitonin, campath, camptothecin, capecitabine, carboplatin, carfilzomib, carmustine, casodex, CCI-779, CDC-501, cediranib, cefesone, celebrex, celmoleukin, cerubidine, cediranib, chlorambucil, cisplatin, cladribine, clodronic acid, clofarabine, colaspase, copanlisib, corixa, crisnatol, crizotinib, cyclophosphamide, cyproterone acetate, cytarabine, dacarbazine, dactinomycin, dasatinib, daunorubicin, daunoxome, decadron, decadron phosphate, decitabin, degarelix, delestrogen, denileukin diftitox, depomedrol, deslorelin, dexrazoxane, diethylstilbestrol, diflucan, 2′,2′-difluorodeoxycytidine, DN-101, docetaxel, doxifluridine, doxorubicin (adriamycin), dronabinol, dSLIM, dutasteride, DW-166HC, edotecarin, eflornithine, eligard, elitek, ellence, emend, enzalutamide, epirubicin, epoetin alfa, epogen, epothilone and derivatives thereof, eptaplatin, ergamisol, erlotinib, erythro-hydroxynonyladenine, estrace, estradiol, estramustine sodium phosphate, ethinylestradiol, ethyol, etidronic acid, etopophos, etoposide, everolimus, exatecan, exemestane, fadrozole, farstone, fenretinide, filgrastim, finasteride, fligrastim, floxuridine, fluconazole, fludarabin, 5-fluorodeoxyuridine monophosphate, 5-fluorouracil (5-FU), fluoxymesterone, flutamide, folotyn, formestane, fosteabine, fotemustine, fulvestrant, gammagard, gefitinib, gemcitabine, gemtuzumab, gleevec, gliadel, goserelin, gossypol, granisetron hydrochloride, hexamethylmelamine, histamine dihydrochloride, histrelin, holmium-166 DOTPM, hycamtin, hydrocortone, erythro-hydroxynonyladenine, hydroxyurea, hydroxyprogesterone caproate, ibandronic acid, ibritumomab tiuxetan, idarubicin, ifosfamide, imatinib, iniparib, interferon-alpha, interferon alpha-2, interferon alpha-2α, interferon alpha-2β, interferon alpha-n1, interferon alpha-n3, interferon beta, interferon gamma-la, interleukin-2, intron A, iressa, irinotecan, ixabepilon, keyhole limpet hemocyanine, kytril, lanreotide, lapatinib, lasofoxifen, lenalidomide, lentinan sulphate, lestaurtinib, letrozole, leucovorin, leuprolide, leuprolide acetate, levamisol, levofolic acid calcium salt, levothroid, levoxyl, libra, liposomal MTP-PE, lomustine, lonafarnib, lonidamine, marinol, mechlorethamine, mecobalamin, medroxyprogesterone acetate, megestrol acetate, melphalan, menest, 6-mercaptopurine, mesna, methotrexate, metvix, miltefosine, minocycline, minodronate, miproxifen, mitomycin C, mitotan, mitoxantrone, modrenal, MS-209, MX-6, myocet, nafarelin, nedaplatin, nelarabine, nemorubicin, neovastat, neratinib, neulasta, neumega, neupogen, nilotimib, nilutamide, nimustine, nolatrexed, nolvadex, NSC-631570, obatoclax, oblimersen, OCT-43, octreotide, olaparib, ondansetron hydrochloride, onko-TCS, orapred, osidem, oxaliplatin, paclitaxel, pamidronate disodium, pazopanib, pediapred, pegaspargase, pegasys, pemetrexed, pentostatin, N-phosphonoacetyl-L-aspartate, picibanil, pilocarpine hydrochloride, pirarubicin, plerixafor, plicamycin, PN-401, porfimer sodium, prednimustine, prednisolone, prednisone, premarin, procarbazine, procrit, QS-21, quazepam, R-1589, raloxifene, raltitrexed, ranpirnas, RDEA119, rebif, regorafenib, 13-cis-retinoic acid, rhenium-186 etidronate, rituximab, roferon-A, romidepsin, romurtide, ruxolitinib, salagen, salinomycin, sandostatin, sargramostim, satraplatin, semaxatinib, semustine, seocalcitol, sipuleucel-T, sizofiran, sobuzoxan, solu-medrol, sorafenib, streptozocin, strontium-89 chloride, sunitinib, synthroid, T-138067, tamoxifen, tamsulosin, tarceva, tasonermin, tastolactone, taxoprexin, taxoter, teceleukin, temozolomide, temsirolimus, teniposide, testosterone propionate, testred, thalidomide, thymosin alpha-1, thioguanine, thiotepa, thyrotropin, tiazorufin, tiludronic acid, tipifarnib, tirapazamine, TLK-286, toceranib, topotecan, toremifen, tositumomab, trastuzumab, teosulphan, transMID-107R, tretinoin, trexall, trimethylmelamine, trimetrexate, triptorelin acetate, triptorelin pamoate, trofosfamide, UFT, uridine, valrubicin, valspodar, vandetanib, vapreotide, vatalanib, vemurafinib, verteporfin, vesnarinone, vinblastine, vincristine, vindesine, vinflumine, vinorelbine, virulizin, vismodegib, xeloda, Z-100, zinecard, zinostatin stimalamer, zofran, zoledronic acid.

More particularly, the compounds according to the invention can be combined with antibodies, for example aflibercept, alemtuzumab, bevacizumab, brentuximumab, catumaxomab, cetuximab, denosumab, edrecolomab, gemtuzumab, ibritumomab, ipilimumab, ofatumumab, panitumumab, pertuzumab, rituximab, tositumumab or trastuzumab, and also with recombinant proteins.

More particularly, the compounds according to the invention can be used in combination with treatments directed against angiogenesis, for example bevacizumab, axitinib, regorafenib, cediranib, sorafenib, sunitinib, lenalidomide, copanlisib or thalidomide.

Combinations with antihormones and steroidal metabolic enzyme inhibitors are particularly suitable because of their favourable profile of side effects.

Combinations with P-TEFb inhibitors and CDK9 inhibitors are likewise particularly suitable because of the possible synergistic effects.

Generally, the following aims can be pursued with the combination of the compounds according to the invention with other cytostatically or cytotoxically active agents:
improved efficacy in slowing the growth of a tumour, in reducing its size or even in completely eliminating it, compared with treatment with an individual active ingredient;
the possibility of using the chemotherapeutics used in a lower dosage than in the case of monotherapy;
the possibility of a more tolerable therapy with fewer side effects compared with individual administration;
the possibility of treatment of a broader spectrum of neoplastic disorders;
the achievement of a higher rate of response to the therapy;
a longer survival time of the patient compared with present-day standard therapy.

The compounds according to the invention can moreover also be employed in combination with radiotherapy and/or surgical intervention.

### Preparation of the Compounds According to the Invention

In the present description:

NMR signals are reported with their respective recognizable multiplicities or combinations thereof. In this context, s=singlet, d=doublet, t=triplet, q=quartet, qi=quintet, sp=septet, m=multiplet, b=broad signal. Signals having combined multiplicities are reported, for example, as dd=doublet of doublets. Chemical shifts 6 are given in ppm (parts per million).
ACN acetonitrile
Ex Example
(+)-BINAP (R)-(+)-2,2′-bis(diphenylphosphino)-1,1′-binaphthyl (CAS 76189-55-4)
(±)-BINAP 2,2′-bis(diphenylphosphino)-1,1′-binaphthyl (racemic, CAS 98327-87-8)
CDCl₃ deuterochloroform
CHAPS 3-{dimethyl[3-(4-{5,9,16-trihydroxy-2,15-dimethyltetracyclo-[8.7.0.02,7.011,15]heptadecan-14-yl}pentanamido)propyl]-azaniumyl}propane-1-sulphonate
DAD diode array detector
dba dibenzylideneacetone
DCC dicyclohexylcarbodiimide
DMF N,N-dimethylformamide
DMSO-d6 deuterated dimethyl sulphoxide
DMSO dimethyl sulphoxide
EA ethyl acetate
FCS fetal calf serum
HATU (7-aza-1H-benzotriazol-1-yl)-1,1,3,3-tetramethyluronium hexafluorophosphate
HEPES 2-{4-(2-hydroxyethyl)-1-piperazinyl}ethanesulphonic acid
HPLC high-pressure liquid chromatography
KOtBu potassium tert-butoxide
LCMS liquid chromatography coupled with mass spectrometry
RP-HPLC reverse-phase high-pressure liquid chromatography
RT room temperature
T3P 2,4,6-tripropyl-1,3,5,2,4,6-trioxatriphosphorinane 2,4,6-trioxide
TBTU (benzotriazol-1-yloxy)bisdimethylaminomethylium fluoroborate
THF Tetrahydrofuran
TFA trifluoroacetic acid
UPLC ultra high performance chromatography
Xanthphos 4,5-bis(diphenylphosphino)-9,9-dimethylxanthene

### General Description of the Preparation of the Compounds of the General Formula (I) According to the Invention

General methods which are used for preparing the compounds of the general formula (I) according to the invention are described below with reference to schemes 1, 2, 3 and 4.

In addition to the synthetic sequences described in the schemes mentioned above, it is also possible, in accordance with the general knowledge of the person skilled in the art in organic chemistry, to take further synthetic routes for the synthesis of compounds of the general formula (I) according to the invention. The sequence of the synthetic steps shown in the schemes which follow is not binding, and synthetic steps from various of the schemes shown hereinafter may optionally be combined to form new sequences. In addition, interconversions of the substituents R¹, R², R³, R⁴, R⁵ and R⁶ can be performed before or after the synthetic stages shown. Examples of such conversions are the introduction or elimination of protective groups, reduction or oxidation of functional groups, reductive amination, halogenation, metallation, metal-catalysed coupling reactions, substitution reactions or further reactions known to the person skilled in the art. These reactions include conversions which introduce a functional group which enables the further conversion of substituents. Suitable protective groups and methods for their introduction and removal are known to the person skilled in the art (see, for example, T. W. Greene and P. G. M. Wuts in: Protective Groups in Organic Synthesis, 3rd edition, Wiley 1999). In addition, it is possible to combine two or more reaction steps without intermediate workup in a manner known to the person skilled in the art (for example in what are called “one-pot” reactions).

Compounds of the general formula (I) and the precursors thereof described hereinafter, in which mutually different substituents R⁴ and R⁵ are present, are chiral and may occur as enantiomer mixtures, for example racemates, or as pure enantiomers. The enantiomer mixtures mentioned can be separated into the enantiomers by separation methods familiar to the person skilled in the art, for example preparative HPLC on a chiral stationary phase.

Scheme 1 illustrates the preparation of intermediates of the formula (VIII) from simple pyridine derivatives such as 3-amino-2,6-dichloropyridine ((II), CAS-No. 62476-56-6). To this end, nitrogen-atom-protected amino acids of the formula (III) in which R⁴ and R⁵ are as defined in the general formula (I) and in which PG represents a protective group such as, for example, Boc, Cbz or else Fmoc, are reacted with suitable aminopyridine derivatives, for example 3-amino-2,6-dichloropyridine ((II), CAS-No. 62476-56-6). Here, use is made of coupling reagents known to the person skilled in the art, such as TBTU, HATU or DCC. The conversion of the carboxylic acids into their amides is described in general terms in reference books such as “Compendium of Organic Synthetic Methods”, volume I-VI (Wiley Interscience) or “The Practice of Peptide Synthesis”, Bodansky (Springer Verlag). Compounds of the formula (III) are known to those skilled in the art and commercially available. The resulting compounds of the formula (IV) are then converted into the compounds of the formula (V) by removing the protective group PG on the amine by suitable methods. A large number of methods suitable for this purpose is known and which can be found in standard references (see, for example, T. W. Greene and P. G. M. Wuts in: Protective Groups in Organic Synthesis, 3rd edition, Wiley 1999). The further conversion to compounds of the formula (VI) with introduction of the R⁶ radical, which is as defined for the general formula (I), can preferably be conducted via the reductive amination known to the person skilled in the art (for representative procedures see, for example, US2010/105906 A1). Here, the primary amine (V), as free base or in salt form, is reacted in situ with an aldehyde or ketone suitable for introducing R⁶ to afford an imine, and the latter is then transformed into the secondary amine of the formula (VI) by addition of a suitable reducing agent such as, for example, sodium triacetoxyborohydride. The secondary amines of the formula (VI) can be converted by cyclization to dihydropyridopyrazinones of the formula (VII). To this end, compounds of the formula (VI) can be reacted in the presence of a suitable base, for example a trialkylamine such as triethylamine, N,N-diisopropylethylamine or N,N-dicyclohexylmethylamine, at elevated temperature (see also WO2010/96426 A2, Example 16). The subsequent alkylation to give compounds of the formula (VIII) can be effected by reaction with R³-LG in which R³ is as defined in the general formula (I) and LG is a leaving group, preferably iodide, in the presence of a suitable base such as sodium hydride, under conditions known to the person skilled in the art.

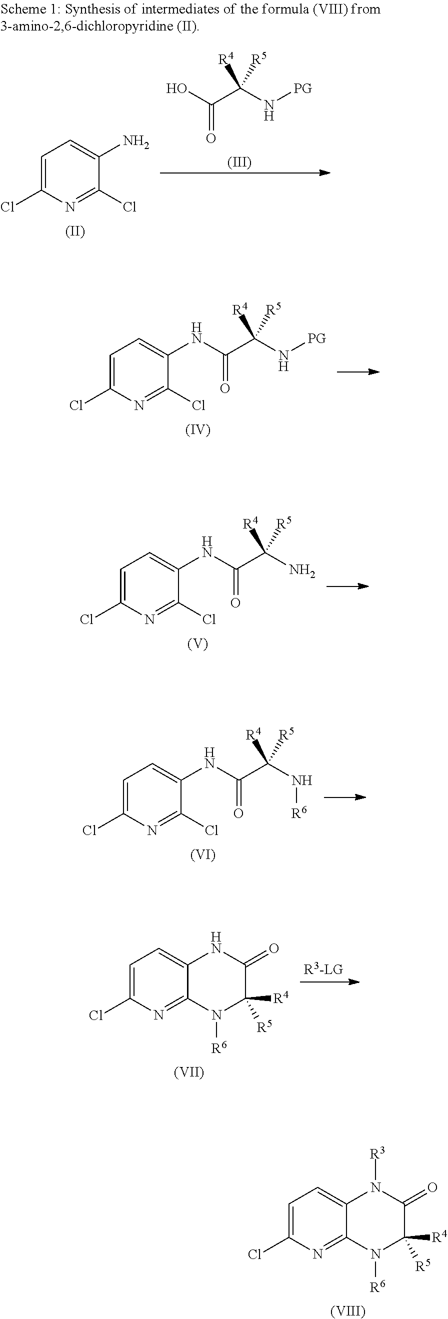

The preparation of intermediates of the formula (VIIa) in which R^{6′} is optionally substituted phenyl as per the definition of R⁶ in the general formula (I) is described in Scheme 2. 3-Amino-2,6-dichloropyridine ((II), CAS No. 62476-56-6) is reacted with compounds of the formula (IX) in which R⁴ and R⁵ are as defined for the general formula (I), and in which LG and LG′ are each independently of one another a leaving group, preferably chlorine or bromine, for example 2-bromopropionyl bromide (CAS 563-76-8). This is done by conversion, under conditions known to the person skilled in the art, with a suitable solvent such as dichloromethane or THF and with addition of a base such as triethylamine, N,N-diisopropylethylamine or pyridine. The base can also be used as the solvent. This gives compounds of the formula (X). These intermediates (X) are reacted with anilines of the formula R^{6′}—NH₂, in which R^{6′} is optionally substituted phenyl as per the definition of R⁶ in the general formula (I), to give compounds of the formula (XI). This reaction can be carried out in various solvents such as toluene or acetonitrile and with addition of a base such as, for example, potassium carbonate, N,N-diisopropylethylamine or triethylamine at elevated temperature (Org. Lett. (2008), 10, S. 2905 ff, S. P. Marsden et al.). Dihydropyridopyrazinones of the formula (VIIa), in which R^{6′} is optionally substituted phenyl as per the definition of R⁶ in the general formula (I), are obtained by cyclizing the compounds of the formula (XI) in the presence of a suitable base, for example triethylamine, N,N-diisopropylethylamine or potassium carbonate, at elevated temperature in solvents such as N,N-dimethylformamide, N,N-dimethylacetamide, N-methylpyrrolidone or else dimethyl sulphoxide (see also WO2010/96426 A2, Example 16). From these intermediates of the formula (VIIa), it is possible according to Schemes 1 and 3 to prepare the corresponding inventive compounds of the formula (I) in which R^{6′} is optionally substituted phenyl as per the definition of R⁶ in the general formula (I). This gives said compounds of the formula (I) as racemates if R⁴ and R⁵ are different from one another. These can optionally be separated into the enantiomers by separation methods familiar to the person skilled in the art, for example preparative HPLC on a chiral stationary phase.

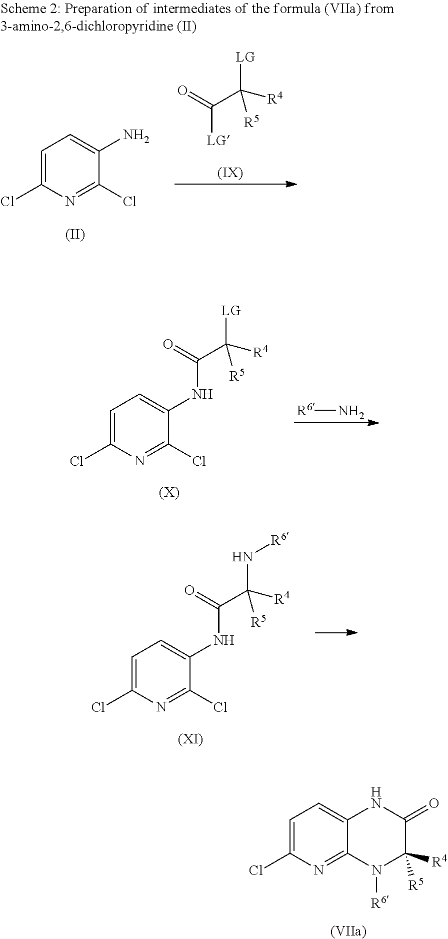

The compounds of the formula (I) according to the invention are prepared according to scheme 3. Here, compounds of the formula (VIII) may be reacted directly with compounds of the formula (XII), in which A, X, Y, R¹, R² and n are defined as in the general formula (I), in a palladium-catalysed coupling reaction according to Buchwald and Hartwig to afford the compounds of the formula (I) according to the invention (e.g. in K. Malagu et al., Bioorg. Med. Chem. Lett., (2009), vol 19, pp 5950-53; P. Fernandez et al. WO2011/101644; see also the synthetic methods described in the experimental section).

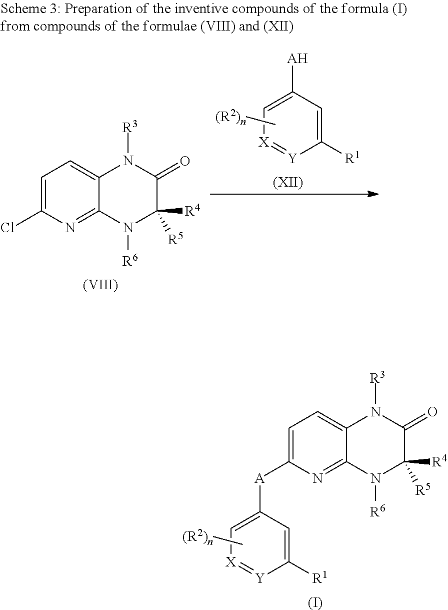

Compounds of the formula (XII) are available for sale in some cases or they themselves or methods suitable for their preparation are known to those skilled in the art.

For example, they can be obtained by reacting commercially available aryl thiols of the formula (XIII) with appropriate substitution, e.g.

4-methoxythiophenol (CAS 696-63-9), with alkyl halides R⁷-Hal for example, in which R⁷ is defined as in the general formula (I), for example iodomethane, bromoethane, bromopropane, 2-bromopropane, cyclopropyl bromide or further homologues thereof, with addition of a base such as sodium, caesium or potassium carbonate, triethylamine or sodium hydride (e.g. analogous to G. Delogu, Tetrahedron Asym., (2001), Vol 12, pp. 3313-17; G. Capozzi et al., J. Org. Chem., (2002), vol. 67, pp. 2019-26). Some aryl thiols can also be obtained in situ from the corresponding sulphonyl chlorides with addition of a reducing agent such as triphenylphosphine (E. V. Bellale, Synthesis (2009), Vol 19, pp. 3211-13).

In this case, compounds of the formula (XIV) are obtained in which X, Y, R², R⁷ and n are defined as in the general formula (I). Compounds of the general formula (XV), in which X, Y, R², R⁷ and n are defined as in the general formula (I), may be obtained by oxidation of the sulphur atom of the compounds of the formula (XIV) using suitable reagents such as potassium peroxosulphate (Oxone®, CAS 70693-62-8), meta-chloroperbenzoic acid or hydrogen peroxide (e.g. analogous to J. M. Zapico, Org. Biomol. Chem., (2011), vol 9, pp. 4587-99).

To prepare compounds of the general formula (XIIa), in which AH is NH₂ and X, Y, R², R⁷ and n are as defined in the general formula (I), a reduction is carried out, known to those skilled in the art, of a compound (XV), in which a radical R² is a nitro group, with hydrogen and a suitable catalyst or using iron, zinc or tin dichloride as reducing agent. Suitable catalysts for the reduction with hydrogen are e.g. palladium or platinum, which may be fixed on various heterogeneous supports such as activated carbon, aluminium oxide or other standard supports, or also Raney nickel for example. The nitro group is reduced using a metal or metal salt frequently with addition of an acid such as hydrochloric acid, acetic acid or ammonium chloride. If no R² group is a nitro group, this can be introduced by nitration of compounds of the formula (XV) under conditions known to those skilled in the art. For this purpose, compounds of the formula (XV) may be dissolved in acids such as sulphuric acid or trifluoroacetic acid and may be converted to compounds of the formula (XVI), in which X, Y, R², R⁷ and n are as defined in the general formula (I), by addition of nitric acid. These can then be converted, as described, to compounds of the formula (XIIa).

Compounds of the general formula (XII), in which A is —N(C₁-C₃-alkyl)- in the corresponding general formula (I), may be obtained by reaction, generally known to those skilled in the art, of compounds of the formula (XIIa) with appropriate aldehydes such as formaldehyde, acetaldehyde or propionaldehyde and a reducing agent such as sodium triacetoxyborohydride or sodium cyanoborohydride or also by reduction with hydrogen and an appropriate catalyst such as palladium on activated carbon.

Compounds of the general formula (XII), in which A is —O— in the corresponding general formula (I), may be obtained by reaction, generally known to those skilled in the art, of compounds of the formula (XIIa) with, for example, sodium nitrite in aqueous acid solution and subsequent heating with a copper catalyst. This reaction is generally known to those skilled in the art as the Sandmeyer reaction.

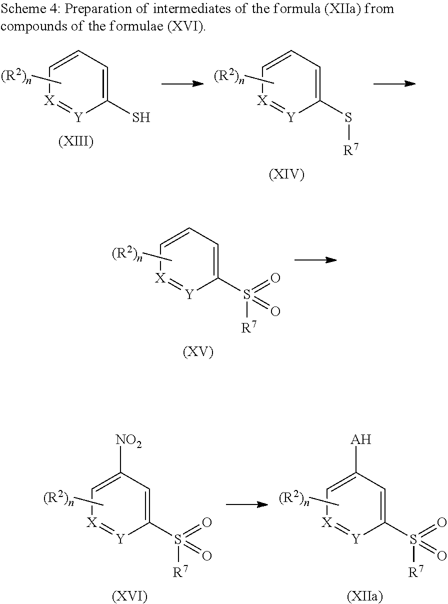

Amines of the general formula (XVII), in which A, X, Y, R², R¹⁰, R¹¹ and n are as defined in the general formula (I), are known to those skilled in the art, in many cases available to purchase or known in the literature, or may be obtained by methods familiar to those skilled in the art.

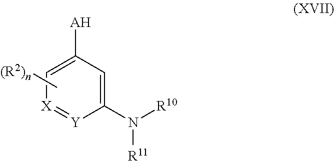

### WORKING EXAMPLES

The examples which follow describe the preparation of the compounds according to the invention, without restricting the invention to these examples.

Firstly, there is a description of the preparation of the intermediates which are ultimately used preferentially for preparation of the compounds according to the invention.

IUPAC names were created with the aid of the nomenclature software ACD Name batch, Version 12.01, from Advanced Chemical Development, Inc., and adapted if required, for example to German-language nomenclature.

### Stoichiometry of Salt Forms

In the case of the synthesis intermediates and working examples of the invention described hereinafter, any compound specified in the form of a salt of the corresponding base or acid is generally a salt of unknown exact stoichiometric composition, as obtained by the respective preparation and/or purification process. Unless specified in more detail, additions to names and structural formulae, such as “hydrochloride”, “trifluoroacetate”, “sodium salt” or “x HCl”, “x CF₃COOH”, “x Na⁺” should not therefore be understood in a stoichiometric sense in the case of such salts, but have merely descriptive character with regard to the salt-forming components present therein.

This applies correspondingly if synthesis intermediates or working examples or salts thereof were obtained in the form of solvates, for example hydrates, of unknown stoichiometric composition (if they are of a defined type) by the preparation and/or purification processes described.

### Preparation of the Intermediates

### Intermediate 1

### N-(2,6-Dichloropyridin-3-yl)-D-alaninamide hydrochloride

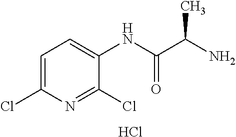

At 0° C., 886 ml of a 50% strength solution of T3P (in ethyl acetate) were added slowly to a solution of 50 g of 3-amino-2,6-dichloropyridine (CAS 62476-56-6) and 56.3 g of D-Boc-alanine in 400 ml of pyridine. The mixture was left stirring at 0° C. for a further 4 hours and at RT for 16 hours. The mixture was added to ice-water, and potassium carbonate was added carefully until the solution was alkaline. The reaction mixture was extracted with ethyl acetate and the organic phase was washed with saturated aqueous sodium chloride solution, dried over sodium sulphate and evaporated to dryness. This gave 73 g of tert-butyl {(2R)-1-[(2,6-dichloropyridin-3-yl)amino]-1-oxopropan-2-yl}carbamate. These were taken up in 370 ml of dioxane, and 89 ml of conc. hydrochloric acid were added at RT. The mixture was stirred at RT for 90 min, 1000 ml of ethyl acetate were added and the pH was adjusted to alkaline using sodium hydroxide. The suspension was decanted, the phases were separated and the organic phase was evaporated to dryness. The residue was taken up in diethyl ether, and 260 ml of 1N HCl (solution in diethyl ether) were added. The mixture was cooled to 0° C. and the precipitate was filtered off with suction. The precipitate was washed with a little diethyl ether and dried in a drying cabinet. This gave 45.6 g of N-(2,6-dichloropyridin-3-yl)-D-alaninamide hydrochloride.

1H-NMR (400 MHz, DMSO-d6): δ=1.50 (d, 3H); 4.23 (bq, 1H); 7.63 (d, 1H); 8.15 (d, 1H); 8.42 bs, 1H); 10.58 (s, 1H).

### Intermediate 2

### N-(2,6-Dichloropyridin-3-yl)-N2-(tetrahydro-2H-pyran-4-yl)-D-alaninamide

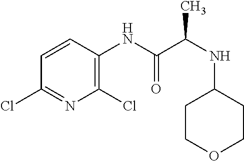 [SMILES: C[C@@H](NC1CCOCC1)C(=O)Nc1ccc(Cl)nc1Cl]

At 0° C., 12.1 g of sodium acetate and 47 g of sodium triacetoxyborohydride were added to a suspension of 20 g of intermediate 1 and 9.6 g tetrahydro-4H-pyran-4-one in 1.07 l of dichloromethane. The mixture was stirred for 16 hours while warming to RT. The reaction was poured carefully into a saturated aqueous sodium hydrogen carbonate solution and stirred. The phases were separated and the aqueous phase was extracted once with dichloromethane. The combined organic phases were dried over sodium sulphate and the solvent was removed completely under reduced pressure. The residue was purified by chromatography on silica gel (hexane/ethyl acetate gradient). This gave 15 g of N-(2,6-dichloropyridin-3-yl)-N2-(tetrahydro-2H-pyran-4-yl)-D-alaninamide.

¹H-NMR (400 MHz, CDCl₃): δ=1.35-1.57 (m, 2H); 1.44 (d, 3H); 1.84 (dq, 1H); 1.95 (dq, 1H); 2.63-2.82 (m, 1H); 3.38 (td, 1H); 3.45 (q, 1H); 3.91-4.08 (m, 2H); 7.28 (d, 1H); 8.84 (d, 1H).

### Intermediate 3

### (3R)-6-Chloro-3-methyl-4-(tetrahydro-2H-pyran-4-yl)-3,4-dihydropyrido[2,3-b]pyrazin-2(1H)-one

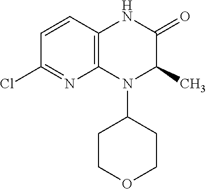 [SMILES: C[C@@H]1C(=O)Nc2ccc(Cl)nc2N1C1CCOCC1]

A solution of 7.8 g of intermediate 2 and 31.7 ml of N,N-diisopropylethylamine in 170 ml of DMF was divided into 4 individual sealed pressure vessels and these were heated at a bath temperature of 175° C. for 10 hours. After cooling to RT, the solutions were re-combined, diluted with ethyl acetate and extracted three times with semisaturated aqueous sodium chloride solution. The organic phase was dried over sodium sulphate and the solvent was removed completely under reduced pressure. The residue was purified by chromatography on silica gel (dichloromethane/methanol gradient). This gave 4.1 g of (3R)-6-chloro-3-methyl-4-(tetrahydro-2H-pyran-4-yl)-3,4-dihydropyrido[2,3-b]pyrazin-2(1H)-one.

¹H-NMR (300 MHz, CDCl₃): δ=1.32 (d, 3H); 1.65 (d, 1H); 1.82 (dq, 1H); 1.98 (dq, 1H); 2.07 (d, 1H); 3.57 (qd, 2H); 4.03-4.12 (m, 2H); 4.25 (q, 1H); 4.55 (tt, 1H); 6.65 (d, 1H); 6.92 (d, 1H); 8.92 (s, 1H).

### Intermediate 4

### (3R)-6-Chloro-1,3-dimethyl-4-(tetrahydro-2H-pyran-4-yl)-3,4-dihydropyrido[2,3-b]pyrazin-2(1H)-one

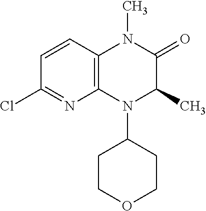 [SMILES: C[C@@H]1C(=O)N(C)c2ccc(Cl)nc2N1C1CCOCC1]

A solution of 3.2 g of intermediate 3, 647 mg of sodium hydride (60% in white oil) and 1.01 ml of methyl iodide in 137 ml of DMF was stirred at RT for 16 hours. The reaction was poured into water and extracted three times with ethyl acetate. The combined organic phases were washed with saturated ammonium chloride solution and semisaturated sodium chloride solution and dried over sodium sulphate, and the solvent was removed completely under reduced pressure. This gave 2.8 g of (3R)-6-chloro-1,3-dimethyl-4-(tetrahydro-2H-pyran-4-yl)-3,4-dihydropyrido[2,3-b]pyrazin-2(1H)-one.

¹H-NMR (300 MHz, CDCl₃): δ=1.24 (d, 3H); 1.66 (dq, 1H); 1.82 (dq, 1H); 1.97 (qd, 1H); 2.06 (dq, 1H); 3.32 (s, 3H); 3.57 (tdd, 2H); 4.01-4.13 (m, 2H); 4.32 (q, 1H); 4.55 (tt, 1H); 6.70 (d, 1H); 7.01 (d, 1H).

chiral HPLC: Rt=5.92 min, (97% ee)

Instrument: Waters Alliance 2695; column: Chiralpak IC 5 μm 100×4.6 mm; mobile phase: hexane/2-propanol 70:30; flow rate 1 ml/min; temperature: 25° C.; injection: 5 μl (1 mg/ml ethanol/methanol, 1:1); DAD 996 scan: 280 nm.

### Intermediate 5

### N²-(1-Methylethyl)-N-(2,6-dichloropyridin-3-yl)-D-alaninamide

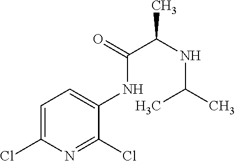 [SMILES: CC(C)N[C@H](C)C(=O)Nc1ccc(Cl)nc1Cl]

Analogously to the preparation of intermediate 2, N²-(1-methylethyl)-N-(2,6-dichloropyridin-3-yl)-D-alaninamide was prepared from 0.5 g of intermediate 1, 0.27 ml of acetone, 303 mg of sodium acetate and 1.18 g of sodium triacetoxyborohydride in 40 ml of dichloromethane at 0° C. This gave 420 mg of N²-(1-methylethyl)-N-(2,6-dichloropyridin-3-yl)-D-alaninamide. This was used directly in the synthesis of the next stage.

¹H-NMR (400 MHz, DMSO-d6): δ=1.02 (d, 3H); 1.05 (d, 3H); 1.27 (d, 3H); 2.77 (sp, 1H); 3.30 (q, 1H); 7.58 (d, 1H); 8.67 (d, 1H).

### Intermediate 6

### (3R)-6-Chloro-3-methyl-4-(propan-2-yl)-3,4-dihydropyrido[2,3-b]pyrazin-2(1H)-one

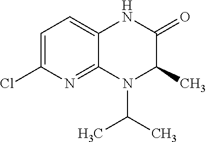 [SMILES: CC(C)N1c2nc(Cl)ccc2NC(=O)[C@H]1C]

Analogously to the synthesis of intermediate 3, (3R)-6-chloro-3-methyl-4-(propan-2-yl)-3,4-dihydropyrido[2,3-b]pyrazin-2(1H)-one was prepared from 420 mg of intermediate 5 and 2.1 ml of N,N-diisopropylethylamine in 40 ml of DMF by heating for 72 hours at a bath temperature of 170° C. This gave 320 mg of (3R)-6-chloro-3-methyl-4-(propan-2-yl)-3,4-dihydropyrido[2,3-b]pyrazin-2(1H)-one.

¹H-NMR (300 MHz, DMSO-d6): δ=1.16 (d, 3H); 1.24 (d, 3H); 1.27 (d, 3H); 4.16 (q, 1H); 4.43 (sp, 1H); 6.65 (d, 1H); 7.00 (d, 1H); 10.56 (s, 1H).

### Intermediate 7

### (3R)-6-Chloro-1,3-dimethyl-4-(propan-2-yl)-3,4-dihydropyrido[2,3-b]pyrazin-2(1H)-one

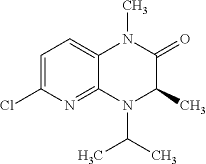 [SMILES: CC(C)N1c2nc(Cl)ccc2N(C)C(=O)[C@H]1C]

Analogously to the preparation of intermediate 4, (3R)-6-chloro-1,3-dimethyl-4-(propan-2-yl)-3,4-dihydropyrido[2,3-b]pyrazin-2(1H)-one was prepared from 320 mg of intermediate 6, 80 mg of sodium hydride (60% in white oil) and 0.13 ml of methyl iodide in 20 ml of DMF. Purification by chromatography on silica gel (hexane/ethyl acetate 2:1) gave 280 mg of (3R)-6-chloro-1,3-dimethyl-4-(propan-2-yl)-3,4-dihydropyrido[2,3-b]pyrazin-2(1H)-one.

¹H-NMR (400 MHz, DMSO-d6): δ=1.12 (d, 3H); 1.23 (d, 3H); 1.27 (d, 3H); 3.22 (s, 3H); 4.32 (q, 1H); 4.47 (sp, 1H); 6.76 (d, 1H); 7.31 (d, 1H).

### Intermediate 8

### N²-Cycloheptyl-N-(2,6-dichloropyridin-3-yl)-D-alaninamide

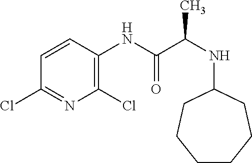 [SMILES: C[C@@H](NC1CCCCCC1)C(=O)Nc1ccc(Cl)nc1Cl]

Analogously to the preparation of intermediate 2, N²-cycloheptyl-N-(2,6-dichloropyridin-3-yl)-D-alaninamide was prepared from 1.5 g of intermediate 1, 809 mg of cycloheptanone, 909 mg of sodium acetate and 3.5 g of sodium triacetoxyborohydride in 80 ml of dichloromethane at 0° C. This gave 1.4 g of N²-cycloheptyl-N-(2,6-dichloropyridin-3-yl)-D-alaninamide.

¹H-NMR (400 MHz, DMSO-d6): δ=1.26 (d, 3H); 1.29-1.42 (m, 4H); 1.42-1.55 (m, 4H); 1.55-1.69 (m, 3H); 1.75-1.88 (m, 2H); 2.56-2.67 (m, 1H); 3.30 (m, 1H); 7.58 (d, 1H); 8.68 (d, 1H).

### Intermediate 9

### (3R)-6-Chloro-4-cycloheptyl-3-methyl-3,4-dihydropyrido[2,3-b]pyrazin-2(1H)-one

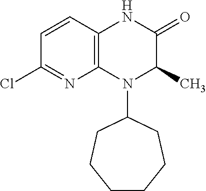 [SMILES: C[C@@H]1C(=O)Nc2ccc(Cl)nc2N1C1CCCCCC1]

Analogously to the synthesis of intermediate 3, (3R)-6-chloro-4-cycloheptyl-3-methyl-3,4-dihydropyrido[2,3-b]pyrazin-2(1H)-one was prepared from 1.4 g of intermediate 8 and 5.77 ml of N,N-diisopropylethylamine in 70 ml of DMF by heating for 72 hours at a bath temperature of 170° C. This gave 1.18 g of (3R)-6-chloro-4-cycloheptyl-3-methyl-3,4-dihydropyrido[2,3-b]pyrazin-2(1H)-one.

¹H-NMR (300 MHz, DMSO-d6): δ=1.16 (d, 3H); 1.37-1.63 (m, 6H); 1.63-2.00 (m, 6H); 3.96-4.09 (m, 1H); 4.17 (q, 1H); 6.64 (d, 1H); 6.98 (d, 1H); 10.57 (s, 1H).

### Intermediate 10

### (3R)-6-Chloro-4-cycloheptyl-1,3-dimethyl-3,4-dihydropyrido[2,3-b]pyrazin-2(1H)-one

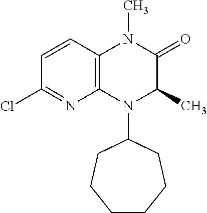 [SMILES: C[C@@H]1C(=O)N(C)c2ccc(Cl)nc2N1C1CCCCCC1]

Analogously to the preparation of intermediate 4, (3R)-6-chloro-4-cycloheptyl-1,3-dimethyl-3,4-dihydropyrido[2,3-b]pyrazin-2(1H)-one was prepared from 1.18 g of intermediate 9, 241 mg of sodium hydride (60% in white oil) and 0.38 ml of methyl iodide in 50 ml of DMF. Purification by chromatography on silica gel (hexane/ethyl acetate 3:1) gave 1.11 g of (3R)-6-chloro-4-cycloheptyl-1,3-dimethyl-3,4-dihydropyrido[2,3-b]pyrazin-2(1H)-one.

¹H-NMR (300 MHz, DMSO-d6): δ=1.13 (d, 3H); 1.38-1.63 (m, 6H); 1.63-1.84 (m, 4H); 1.83-2.03 (m, 2H); 3.21 (s, 3H); 4.00-4.14 (m, 1H); 4.32 (q, 1H); 6.75 (d, 1H); 7.29 (d, 1H).

### Intermediate 11

### N²-(1-Benzylpiperidin-4-yl)-N-(2,6-dichloropyridin-3-yl)-D-alaninamide

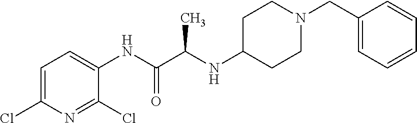 [SMILES: C[C@@H](NC1CCN(Cc2ccccc2)CC1)C(=O)Nc1ccc(Cl)nc1Cl]

A solution of 10 g of intermediate 1 and 8.89 g of 1-benzylpiperidone (CAS 3612-20-2) in 100 ml of dichloromethane was admixed at RT with 18.2 g of sodium triacetoxyborohydride. After 16 hours, the mixture was poured cautiously onto saturated sodium hydrogencarbonate solution, the phases were separated and the aqueous phase was extracted with dichloromethane. The combined organic phases were dried over sodium sulphate and concentrated under reduced pressure. The residue was purified by chromatography on silica gel (heptane/ethyl acetate gradient). This gave 15.1 g of N²-(1-benzylpiperidin-4-yl)-N-(2,6-dichloropyridin-3-yl)-D-alaninamide.

¹H NMR (400 MHz, 25° C., CDCl₃): δ=1.17 (bs, 1H), 1.37-1.52 (m, 5H), 1.86 (d, 1H), 1.91-2.04 (m, 3H), 2.48 (bs, 1H), 2.83-2.88 (m, 2H), 3.38 (q, 1H), 3.51 (s, 2H), 7.22-7.33 (m, 6H), 8.82 (d, 1H), 10.4 (bs, 1H).

### Intermediate 12

### (3R)-4-(1-Benzylpiperidin-4-yl)-6-chloro-3-methyl-3,4-dihydropyrido[2,3-b]pyrazin-2(1H)-one

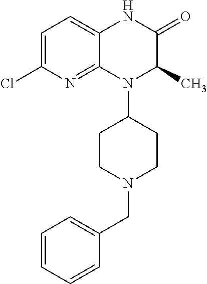 [SMILES: C[C@@H]1C(=O)Nc2ccc(Cl)nc2N1C1CCN(Cc2ccccc2)CC1]

A solution of 15.1 g of intermediate 11 and 32.3 ml of N,N-diisopropylethylamine in 277 ml of DMA was stirred in a tightly sealed vessel at bath temperature 170° C. for 48 hours. After cooling, the mixture was diluted with water and extracted three times with ethyl acetate. The combined organic phases were concentrated under reduced pressure. Toluene was added, and the mixture was concentrated fully under reduced pressure once more. The residue was stirred in a heptane/water mixture, and the precipitate was filtered off with suction and then dried by distillation with toluene. This gave 13.8 g of (3R)-4-(1-benzylpiperidin-4-yl)-6-chloro-3-methyl-3,4-dihydropyrido[2,3-b]pyrazin-2(1H)-one.

¹H NMR (400 MHz, 25° C., CDCl₃): δ=1.27 (d, 3H), 1.54-1.81 (m, 3H), 1.86-2.26 (m, 3H), 2.90-3.05 (m, 2H), 3.54 (s, 2H), 4.22-4.39 (m, 2H), 6.60 (d, 1H), 6.87 (d, 1H), 7.25-7.32 (m, 5H), 8.72 (bs, 1H).

### Intermediate 13

### (R)-4-(1-Benzylpiperidin-4-yl)-6-chloro-1,3-dimethyl-3,4-dihydropyrido[2,3-b]pyrazin-2(1H)-one

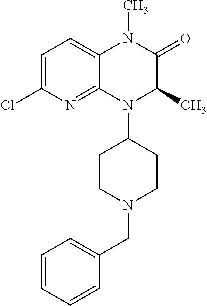 [SMILES: C[C@@H]1C(=O)N(C)c2ccc(Cl)nc2N1C1CCN(Cc2ccccc2)CC1]

A solution of 13.1 g of intermediate 12 in 131 ml of DMF was admixed at 0° C. with 2.08 mg of sodium hydride (60% in white oil) in portions. The mixture was stirred at RT for another 30 min, then cooled again to 0° C., and 2.28 ml of methyl iodide were added. After about 10 min, the mixture was added rapidly to ice-water under an argon atmosphere, and the precipitate was filtered off with suction and washed with heptane. This gave 12.7 g of (R)-4-(1-benzylpiperidin-4-yl)-6-chloro-1,3-dimethyl-3,4-dihydropyrido[2,3-b]pyrazin-2(1H)-one.

¹H NMR (400 MHz, 25° C., CDCl₃): δ=1.19 (d, 3H), 1.57-1.79 (m, 2H+H₂O), 1.92 (bq, 1H), 2.04-2.22 (m, 3H), 2.96 (bs, 2H), 3.28 (s, 3H), 3.54 (s, 2H), 4.30-4.35 (m, 2H), 6.65 (d, 1H), 6.96 (d, 1H), 7.31-7.37 (m, 5H).

### Intermediate 14

### (3R)-6-Chloro-1,3-dimethyl-4-(piperidin-4-yl)-3,4-dihydropyrido[2,3-b]pyrazin-2(1H)-one hydrochloride

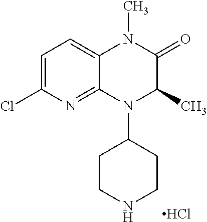 [SMILES: C[C@@H]1C(=O)N(C)c2ccc(Cl)nc2N1C1CCNCC1 ; Cl]

A solution of 12.2 g of intermediate 13 and 4.46 ml of 1-chlorethyl carbonochloridate (CAS 50893-53-3) in 131 ml of 1,2-dichloroethane was heated under reflux for 4 hours. The mixture was concentrated fully and taken up in ethyl acetate/heptane (1:1). This solution was filtered through silica gel and washed first with heptane, then with ethyl acetate. The eluted residue was heated in methanol and then concentrated again. This gave 8.2 g of (3R)-6-chloro-1,3-dimethyl-4-(piperidin-4-yl)-3,4-dihydropyrido[2,3-b]pyrazin-2(1H)-one hydrochloride.

¹H NMR (400 MHz, 25° C., DMSO-d6₃): δ=1.22 (d, 3H), 1.94-2.01 (m, 1H), 2.13 (dq, 1H), 2.23-2.37 (m, 2H), 3.16 (tt, 2H), 3.30 (s, 3H), 3.43-3.53 (m, 2H), 4.28 (q, 1H), 4.39 (tt, 1H), 6.80 (d, 1H), 7.07-7.21 (m, 1H), 7.32 (d, 1H).

### Intermediate 15

### (3R)-6-Chloro-1,3-dimethyl-4-(1-methylpiperidin-4-yl)-3,4-dihydropyrido[2,3-b]pyrazin-2(1H)-one

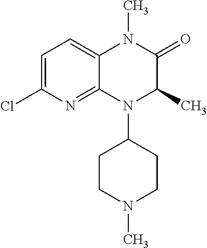 [SMILES: C[C@@H]1C(=O)N(C)c2ccc(Cl)nc2N1C1CCN(C)CC1]

A solution of 8.2 g of intermediate 14 in 77.1 ml of methanol was admixed at RT first with 77.1 ml of formaldehyde solution (37% in water) and then with 2.19 g of sodium cyanoborohydride and 3.49 g of acetic acid. The mixture was stirred for 16 hours, and then 2 N sodium hydroxide solution was added. The reaction solution was extracted with ethyl acetate, the organic phase was dried over sodium sulphate and the solvent was removed under reduced pressure. The residue was purified by chromatography on silica gel (start at heptane/ethyl acetate 1:1 with gradient to ethyl acetate/triethylamine/methanol 92:5:3). This gave 6.7 g of (3R)-6-chloro-1,3-dimethyl-4-(1-methylpiperidin-4-yl)-3,4-dihydropyrido[2,3-b]pyrazin-2(1H)-one.

¹H NMR (400 MHz, 25° C., CDCl₃): δ=1.20 (d, 3H), 1.62-1.68 (m, 1H), 1.75 (dq, 1H), 1.95 (dq, 1H), 2.07-2.21 (m, 3H), 2.31 (s, 3H), 2.94 (d, 2H), 3.29 (s, 3H), 4.25-4.35 (m, 2H), 6.66 (d, 1H), 6.97 (d, 1H).

### Intermediate 16

### 2-Bromo-N-(2,6-dichloropyridin-3-yl)propanamide

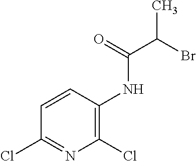

At RT, 20.3 g of 2-bromopropionyl bromide (CAS 563-76-8) were added slowly to a solution of 8.5 g of 3-amino-2,6-dichloropyridine (CAS 62476-56-6) in 200 ml of THF and 12.7 ml of pyridine. The mixture was left stirring at RT for 72 hours. Water was then added, and the mixture was extracted with ethyl acetate. The organic phase was dried over sodium sulphate and concentrated completely under reduced pressure. The residue was purified by chromatography on silica gel (dichloromethane). This gave 8.2 g of 2-bromo-N-(2,6-dichloropyridin-3-yl)propanamide.

¹H-NMR (300 MHz, DMSO-d6): δ=1.76 (d, 3H); 4.94 (q, 1H); 7.60 (d, 1H); 8.22 (d, 1H); 10.17 (s, 1H).

### Intermediate 17

### N-(2,6-Dichloropyridin-3-yl)-N2-phenylalaninamide

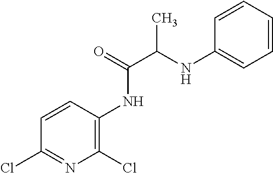 [SMILES: CC(Nc1ccccc1)C(=O)Nc1ccc(Cl)nc1Cl]

A solution of 2.7 g of intermediate 16 and 759 mg of aniline in 27 ml of toluene and 2.7 ml of diisopropylethylamine was stirred at 140° C. for 3 hours. After cooling to RT, water was added and the mixture was extracted with ethyl acetate. The organic phase was dried over sodium sulphate and concentrated fully under reduced pressure. The residue was purified by chromatography on silica gel (dichloromethane). This gave 3.1 g of N-(2,6-dichloropyridin-3-yl)-N2-phenylalaninamide which was sufficiently pure for further reactions.

¹H-NMR (300 MHz, DMSO-d6): δ=1.44 (d, 3H); 4.12 (qi, 1H); 6.11 (d, 1H); 6.64 (d, 2H); 6.99 (t, 1H); 7.10 (t, 2H); 7.56 (d, 1H); 8.29 (d, 1H); 9.79 (s, 1H).

### Intermediate 18

### 6-Chloro-3-methyl-4-phenyl-3,4-dihydropyrido[2,3-b]pyrazin-2(1H)-one

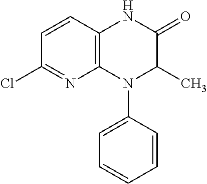 [SMILES: CC1C(=O)Nc2ccc(Cl)nc2N1c1ccccc1]

Analogously to the synthesis of intermediate 3, 6-chloro-3-methyl-4-phenyl-3,4-dihydropyrido[2,3-b]pyrazin-2(1H)-one was prepared from 1.8 g of intermediate 17 and 12.3 ml of N,N-dicyclohexylmethylamine in 10 ml of DMF by heating for 18 hours at a bath temperature of 170° C. This gave 350 mg of 6-chloro-3-methyl-4-phenyl-3,4-dihydropyrido[2,3-b]pyrazin-2(1H)-one.

¹H-NMR (300 MHz, DMSO-d6): δ=1.29 (d, 3H); 4.48 (q, 1H); 6.84 (d, 1H); 7.17 (d, 1H); 7.22 (t, 1H); 7.33 (d, 2H); 7.41 (t, 2H); 10.82 (s, 1H).

### Intermediate 19

### 6-Chloro-1,3-dimethyl-4-phenyl-3,4-dihydropyrido[2,3-b]pyrazin-2(1H)-one

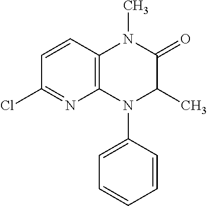 [SMILES: CC1C(=O)N(C)c2ccc(Cl)nc2N1c1ccccc1]

Analogously to the preparation of intermediate 4, 6-chloro-1,3-dimethyl-4-phenyl-3,4-dihydropyrido[2,3-b]pyrazin-2(1H)-one was prepared from 500 mg of intermediate 18 (obtained from 2 reactions), 120 mg of sodium hydride (60% in white oil) and 0.171 ml of methyl iodide in 9 ml of DMF. Chromatography on silica gel (hexane/ethyl acetate gradient) gave 380 mg of 6-chloro-1,3-dimethyl-4-phenyl-3,4-dihydropyrido[2,3-b]pyrazin-2(1H)-one.

¹H-NMR (300 MHz, DMSO-d6): δ=1.29 (d, 3H); 3.32 (s, 3H); 4.60 (q, 1H); 6.96 (d, 1H); 7.21 (t, 1H); 7.33 (d, 2H); 7.41 (t, 2H); 7.50 (d, 1H).

### Intermediate 20

### Cyclopropyl 3-nitrophenyl sulphide

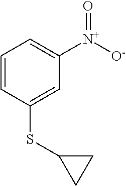 [SMILES: O=[N+]([O-])c1cccc(SC2CC2)c1]

A total of 35.5 g of triphenylphosphine were added portionwise to a solution of 10.0 g of 3-nitrobenzenesulphonyl chloride in 100 ml of toluene. On completion of addition, stirring was continued at RT for 2 h. The mixture was then diluted with 100 ml of N,N-dimethylformamide and 29.4 g of caesium carbonate and 16.4 g of cyclopropyl bromide were added and the mixture heated under reflux for 36 hours. After cooling to RT, water was added and the mixture was extracted twice with ethyl acetate. The combined organic phases were dried over magnesium sulphate and the solvent was removed under reduced pressure. The residue was purified by chromatography on silica gel (heptane/ethyl acetate gradient up to 10% ethyl acetate content). This gave 5.12 g of cyclopropyl 3-nitrophenyl sulphide.

¹H-NMR (300 MHz, 25° C., CDCl₃): δ=0.71-0-78 (m, 2H); 1.16-1.23 (m, 2H); 2.21-2.28 (m, 1H); 7.44 (t, 1H); 7.62 (ddd, 1H); 7.96 (ddd, 1H); 8.22 (t, 1H).

### Intermediate 21

### Cyclopropyl 3-nitrophenyl sulphone

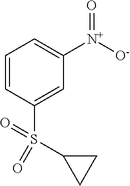 [SMILES: O=[N+]([O-])c1cccc(S(=O)(=O)C2CC2)c1]

A total of 20.15 g of potassium peroxymonosulfate (Oxone®, CAS 70693-62-8) were added portionwise at 0-5° C. to a solution of 5.12 g of intermediate 20 in 50 ml of acetone, 5.1 ml of water and 5.1 ml of methanol and the mixture was then stirred at RT for 72 hours. The mixture was diluted with ethyl acetate, the solid was filtered off and the precipitate washed with ethyl acetate. The combined organic phases were concentrated to dryness under reduced pressure and the residue was taken up in ethyl acetate. The organic phase was washed with saturated aqueous sodium chloride solution, dried over magnesium sulphate and the solvent was completely removed under reduced pressure. The residue was triturated with diethyl ether and filtered off with suction. This gave 2.17 g of cyclopropyl 3-nitrophenyl sulphone.

¹H-NMR (300 MHz, 25° C., CDCl₃): δ=1.09-1.18 (m, 2H); 1.40-1.48 (m, 2H); 2.49-2.57 (m, 1H); 7.82 (t, 1H); 8.25 (bd, 1H); 8.51 (ddd, 1H); 8.76 (t, 1H).

### Intermediate 22

### 3-(Cyclopropylsulphonyl)aniline

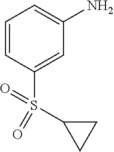 [SMILES: Nc1cccc(S(=O)(=O)C2CC2)c1]

A suspension of 2.17 g of intermediate 21 and 2.67 g of iron powder in 25 ml of ethanol and 6.8 ml of saturated aqueous ammonium chloride solution was stirred at reflux temperature for 5 hours. The mixture was filtered through kieselguhr and washed through with ethyl acetate. Water was added and the organic solvent was removed under reduced pressure. The remaining aqueous solution was extracted three times with ethyl acetate, the combined organic phases were washed with saturated aqueous sodium chloride solution, dried over sodium sulphate and the solvent was removed fully under reduced pressure. The residue was triturated with diethyl ether and filtered off with suction. This gave 1.43 g of 3-(cyclopropylsulphonyl)aniline.

¹H-NMR (300 MHz, 25° C., CDCl₃): δ=0.98-1.07 (m, 2H); 1.30-1.37 (m, 2H); 2.42-2.50 (m, 1H); 3.97 (bs, 2H); 6.89 (ddd, 1H); 7.17 (t, 1H); 7.24 (ddd, 1H); 7.32 (t, 1H).

### Intermediate 23

### 1-(Cyclopropylsulphanyl)-4-methoxybenzene

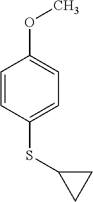

25.9 g of cyclopropyl bromide were added to a suspension of 15 g of 4-methoxythiophenol and 52.3 g of caesium carbonate in 105 ml of N,N-dimethylformamide and the mixture was stirred with heating at 60° C. for 16 hours. After cooling to RT, the mixture was poured into water and extracted twice with ethyl acetate. The combined organic phases were washed with saturated aqueous sodium chloride solution and the solvent was removed under reduced pressure. The residue was purified by chromatography on silica gel (heptane/ethyl acetate gradient up to 10% ethyl acetate content). This gave 19.2 g of 1-(cyclopropylsulphanyl)-4-methoxybenzene.

¹H-NMR (300 MHz, 25° C., CDCl₃): δ=0.66-0.71 (m, 2H); 0.96-1.02 (m, 2H); 2.15-2.23 (m, 1H); 3.81 (s, 3H); 6.87 (d, 2H); 7.35 (d, 2H).

### Intermediate 24

### Cyclopropyl 4-methoxyphenyl sulphone

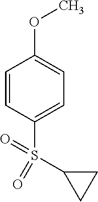

A total of 52.38 g of potassium peroxymonosulphate (Oxone®, CAS 70693-62-8) were added portionwise at 0-5° C. to a solution of 19.2 g of intermediate 23 in 192 ml of acetone, 19.2 ml of water and 19.2 ml of methanol and the mixture was then stirred at RT for 16 hours. The mixture was diluted with ethyl acetate, the solid was filtered off and the precipitate washed with ethyl acetate. The combined organic phases were concentrated to dryness under reduced pressure and the residue was taken up in ethyl acetate. The organic phase was washed with saturated aqueous sodium chloride solution, dried over magnesium sulphate and the solvent was completely removed under reduced pressure. This gave 16.7 g of cyclopropyl 4-methoxyphenyl sulphone.

¹H-NMR (300 MHz, 25° C., CDCl₃): δ=0.97-1.04 (m, 2H); 1.29-1.35 (m, 2H); 2.40-2.48 (m, 1H); 3.89 (s, 3H); 7.01 (d, 2H); 7.82 (d, 2H).

### Intermediate 25

### Cyclopropyl 4-methoxy-3-nitrophenyl sulphone

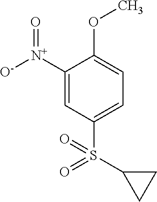 [SMILES: COc1ccc(S(=O)(=O)C2CC2)cc1[N+](=O)[O-]]

2.3 ml of concentrated nitric acid were added dropwise to a solution of 20 g of intermediate 24 (prepared from two batches of intermediate 24) in 100 ml of concentrated sulphuric acid such that the temperature remained between 20-30° C. After further stirring for 15 min. at RT, the mixture was added to ice-water and extracted twice with ethyl acetate. The combined organic phases were washed with saturated aqueous sodium chloride solution, dried over magnesium sulphate and the solvent was completely removed under reduced pressure. This batch was repeated once again with 10 g of intermediate 24. The combined residues of the two batches were taken up in 200 ml of heptane and the solution was concentrated again under reduced pressure to ca. 100 ml. Crystallization occurred here. The mixture was left to stand at RT and further crystallization occurred. The latter was filtered with suction and washed with heptane. This gave 15.8 g of cyclopropyl 4-methoxy-3-nitrophenyl sulphone.

¹H-NMR (300 MHz, 25° C., CDCl₃): δ=1.06-1.13 (m, 2H); 1.35-1.42 (m, 2H); 2.44-2.53 (m, 1H); 4.08 (s, 3H); 7.26 (d, 1H); 8.07 (dd, 1H); 8.37 (d, 1H).

### Intermediate 26

### 5-(Cyclopropylsulphonyl)-2-methoxyaniline

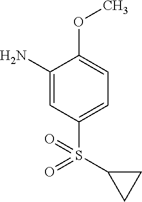

A suspension of 10 g of intermediate 25 and 10.85 g of iron powder in 100 ml of ethanol and 27.7 ml of saturated aqueous ammonium chloride solution was stirred with heating at reflux temperature for 2 hours. The mixture was filtered through kieselguhr and washed through with ethanol. The organic solvent was removed under reduced pressure. The remaining aqueous solution was diluted with water, extracted twice with ethyl acetate, the combined organic phases were washed with saturated aqueous sodium chloride solution, dried over magnesium sulphate and the solvent was removed fully under reduced pressure. The residue was crystallized from heptane/ethyl acetate 1:1. This gave 6.3 g of 5-(cyclopropylsulphonyl)-2-methoxyaniline.

¹H-NMR (300 MHz, 25° C., CDCl₃): δ=0.95-1.03 (m, 2H); 1.27-1.34 (m, 2H); 2.40-2.48 (m, 1H); 3.93 (s, 3H); 4.05 (bs, 2H); 6.87 (d, 1H); 7.18 (d, 1H); 7.27 (dd, 1H).

### Intermediate 27

### 1-(Isopropylsulphanyl)-4-methoxybenzene

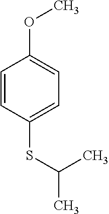

38 ml of sodium methoxide solution in methanol (506 M) and 26.8 ml of 2-bromopropane were added to a suspension of 20 g of 4-methoxythiophenol in 100 ml of methanol and the mixture was stirred with heating at 60° C. for 2 hours. After cooling to RT, the mixture was poured into water and extracted twice with ethyl acetate. The combined organic phases were concentrated under reduced pressure. The residue was purified by chromatography on silica gel (heptane/ethyl acetate gradient up to 10% ethyl acetate content). This gave 24.2 g of 1-(isopropylsulphanyl)-4-methoxybenzene.

¹H-NMR (300 MHz, 25° C., CDCl₃): δ=1.24 (d, 6H); 3.19 (sept, 1H); 3.81 (s, 3H); 6.85 (d, 2H); 7.40 (d, 2H).

### Intermediate 28

### Isopropyl 4-methoxyphenyl sulphone

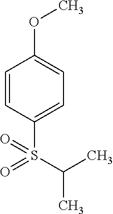

A total of 64.8 g of potassium peroxymonosulphate (Oxone®, CAS 70693-62-8) were added portionwise at RT to a solution of 24 g of intermediate 28 in 240 ml of acetone, 24 ml of water and 24 ml of methanol and the mixture was then stirred at RT for 1 hour. A further 32.4 g of potassium peroxymonosulphate (Oxone®, CAS 70693-62-8) were then added and the mixture was stirred at RT for 16 hours. The mixture was diluted with ethyl acetate, the solid was filtered off and the precipitate washed with ethyl acetate. The combined organic phases were concentrated to dryness under reduced pressure and the residue was taken up in ethyl acetate. The organic phase was washed with saturated aqueous sodium chloride solution, dried over magnesium sulphate and the solvent was completely removed under reduced pressure. This gave 27.2 g of isopropyl 4-methoxyphenyl sulphone.

¹H-NMR (300 MHz, 25° C., CDCl₃): δ=1.28 (d, 6H); 3.15 (sept, 1H); 3.88 (s, 3H); 7.02 (d, 2H); 7.79 (d, 2H).

### Intermediate 29

### Isopropyl 4-methoxy-3-nitrophenyl sulphone

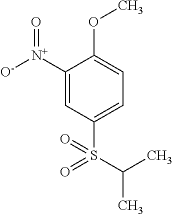 [SMILES: COc1ccc(S(=O)(=O)C(C)C)cc1[N+](=O)[O-]]

4.9 ml of concentrated nitric acid were added dropwise to a solution of 21 g of intermediate 28 in 105 ml of concentrated sulphuric acid such that the temperature remained between 20-30° C. After further stirring for 15 min. at RT, the mixture was added to ice-water and extracted twice with ethyl acetate. The combined organic phases were concentrated to dryness under reduced pressure. The residue was purified by chromatography on silica gel (heptane/ethyl acetate gradient up to 50% ethyl acetate content). This gave 23.3 g of isopropyl 4-methoxy-3-nitrophenyl sulphone.

¹H-NMR (300 MHz, 25° C., CDCl₃): δ=1.33 (d, 6H); 3.22 (sept, 1H); 4.08 (s, 3H); 7.27 (d, 1H); 8.05 (dd, 1H); 8.34 (d, 1H).

### Intermediate 30

### 5-(Isopropylsulphonyl)-2-methoxyaniline

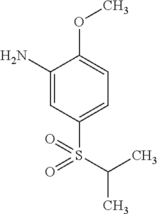

A suspension of 22.2 g of intermediate 29 and 23.9 g of iron powder in 222 ml of ethanol and 61.2 ml of saturated aqueous ammonium chloride solution was stirred with heating at reflux temperature for 2 hours. The mixture was filtered through kieselguhr and washed through with ethanol. The organic solvent was removed under reduced pressure. The remaining aqueous solution was diluted with water, extracted twice with ethyl acetate, the combined organic phases were washed with saturated aqueous sodium chloride solution, dried over magnesium sulphate and the solvent was removed fully under reduced pressure. The residue was crystallized from heptane/ethyl acetate 1:1. This gave 13.7 g of 5-(isopropylsulphonyl)-2-methoxyaniline.

¹H-NMR (300 MHz, 25° C., CDCl₃): δ=1.28 (d, 6H); 3.15 (sept, 1H); 3.93 (s, 3H); 4.04 (bs, 2H); 6.87 (d, 1H); 7.15 (d, 1H); 7.25 (dd, 1H).

### Intermediate 31

### 4-(Isopropylsulphanyl)phenyl trifluoromethyl ether

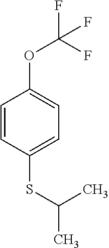

4.2 ml of a sodium methoxide solution (30% in methanol) was added cautiously at RT to a solution of 3 g of 4-trifluoromethoxythiophenol (CAS 169685-29-4) in 10 ml of methanol. The mixture was stirred for 10 min and 2.8 ml of 2-bromopropane were then added. This mixture was stirred for 3 hours at a bath temperature of 60° C. and 14 hours at RT. The mixture was added to water and extracted three times with ethyl acetate. The combined organic phases were washed with saturated sodium chloride solution and water, dried over sodium sulphate, and the solvent was removed completely under reduced pressure. This gave 3.0 g of 4-(isopropylsulphanyl)phenyl trifluoromethyl ether.

¹H-NMR: (400 MHz, 25° C., DMSO-d6): δ=1.24 (d, 6H); 3.52 (sp, 1H); 7.32 (d, 2H); 7.47 (d, 2H).

### Intermediate 32

### 1-(Isopropylsulphonyl)-4-(trifluoromethoxy)benzene

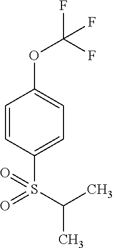 [SMILES: CC(C)S(=O)(=O)c1ccc(OC(F)(F)F)cc1]

A total of 6.2 g of potassium peroxymonosulphate (Oxone®, CAS 70693-62-8) were added portionwise at RT to a mixture of 3 g of intermediate 31 in 24 ml of acetone, 2.4 ml of water and 2.4 ml of methanol and the mixture was then stirred at RT for 5 hours. A further 3.1 g of potassium peroxymonosulphate (Oxone®, CAS 70693-62-8) were then added and the mixture was stirred at RT for 16 hours. The mixture was diluted with ethyl acetate, the solid was filtered off and the precipitate washed with ethyl acetate. The combined organic phases were concentrated to dryness under reduced pressure and the residue was taken up in ethyl acetate. The organic phase was washed with saturated aqueous sodium chloride solution, dried over magnesium sulphate and the solvent was completely removed under reduced pressure. This gave 3.1 g of 1-(isopropylsulphonyl)-4-(trifluoromethoxy)benzene.

¹H-NMR (400 MHz, 25° C., DMSO-d6): δ=1.16 3.49 (sp, 1H); 7.66 (d, 2H); 8.00 (d, 2H).

### Intermediate 33

### Isopropyl 3-nitro-4-(trifluoromethoxy)phenyl sulphone

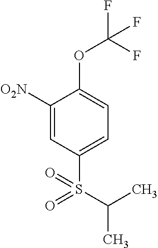 [SMILES: CC(C)S(=O)(=O)c1ccc(OC(F)(F)F)c([N+](=O)[O-])c1]

0.82 ml of concentrated nitric acid (65%) was added dropwise at 0° C. to a mixture of 3 g of intermediate 32 in 11.7 ml of concentrated sulphuric acid. The mixture was stirred at RT for 14 hours. The mixture was added to ice-water and extracted twice with ethyl acetate. The combined organic phases were washed with saturated sodium chloride solution and the solvent concentrated fully under reduced pressure. This gave 3.2 g of isopropyl 3-nitro-4-(trifluoromethoxy)phenyl sulphone.

¹H-NMR (400 MHz, 25° C., DMSO-d6): δ=1.20 (d, 6H); 3.65 (sp, 1H); 7.99-8.05 (m, 1H); 8.32 (dd, 1H); 8.59 (d, 1H).

### Intermediate 34

### 5-(Isopropylsulphonyl)-2-(trifluoromethoxy)aniline

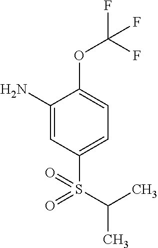 [SMILES: CC(C)S(=O)(=O)c1ccc(OC(F)(F)F)c(N)c1]

A mixture of 3 g of intermediate 33 and 300 mg of palladium on carbon (10%) in 47 ml of methanol was shaken at RT under a 1 bar hydrogen atmosphere for 5 hours. The mixture was filtered through kieselguhr and the solution was fully concentrated. The residue was purified by chromatography on modified silica gel (Biotage column KP—NH, eluent: dichloromethane/methanol gradient to 0.5% methanol). This gave 1.8 g of 5-(isopropylsulphonyl)-2-(trifluoromethoxy)aniline.

¹H-NMR (400 MHz, 25° C., DMSO-d6): δ=1.15 (d, 6H); 3.34 (sp, 1H); 6.00 (s, 2H); 6.97 (dd, 1H); 7.29 (d, 1H); 7.34-7.39 (m, 1H).

### Intermediate 35

### 4-[(4-Methoxyphenyl)sulphanyl]tetrahydro-2H-pyran

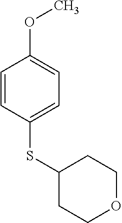

A mixture of 3.5 g of 4-methoxythiophenol (CAS 696-63-9) and 4.7 g of 4-bromotetrahydro-2H-pyran (CAS 25637-16-5) and 9 g of caesium carbonate in 46 ml of DMF was stirred for 6 hours at a bath temperature of 60° C. The mixture was added to water and extracted with ethyl acetate. The organic phase was washed five times with semi-saturated sodium chloride solution, dried over sodium sulphate and the solvent was completely removed under reduced pressure. This gave 5.2 g of 4-[(4-methoxyphenyl)sulphanyl]tetrahydro-2H-pyran as a yellow oil.

¹H-NMR: (400 MHz, 25° C., DMSO-d6): δ=1.36-1.48 (m, 2H); 1.71-1.80 (m, 2H); 3.19 (tt, 1H); 3.31 (dt, 2H); 3.75 (s, 3H); 3.81 (td, 2H); 6.92 (d, 2H); 7.38 (d, 2H).

### Intermediate 36

### 4-[(4-Methoxyphenyl)sulphonyl]tetrahydro-2H-pyran

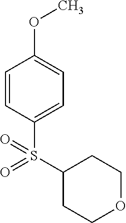 [SMILES: COc1ccc(S(=O)(=O)C2CCOCC2)cc1]

A total of 27.3 g of potassium peroxymonosulphate (Oxone®, CAS 70693-62-8) were added portionwise at RT to a solution of 4.2 g of intermediate 35 in 127 ml of methanol and the mixture was then stirred at RT for 14 hours. The mixture was concentrated under reduced pressure and the residue was taken up in dichloromethane. The mixture was washed with 1 N hydrochloric acid and saturated aqueous sodium chloride solution, dried over sodium sulphate, and the solvent was removed completely under reduced pressure. This gave 4.7 g of 4-[(4-methoxyphenyl)sulphonyl]tetrahydro-2H-pyran.

¹H-NMR (400 MHz, 25° C., DMSO-d6): δ=1.49 (qd, 2H); 1.67-1.75 (m, 2H); 3.26 (dt, 2H); 3.43 (tt, 1H); 3.85-3.92 (m, 5H); 7.18 (d, 2H); 7.76 (d, 2H).

### Intermediate 37

### 4-[(4-Methoxy-3-nitrophenyl)sulphonyl]tetrahydro-2H-pyran

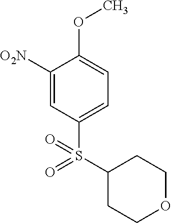 [SMILES: COc1ccc(S(=O)(=O)C2CCOCC2)cc1[N+](=O)[O-]]

1.3 ml of concentrated nitric acid (65%) was added dropwise at 0° C. to a mixture of 4.5 g of intermediate 36 in 18.4 ml of concentrated sulphuric acid. The mixture was stirred for 10 min at 0° C. The mixture was added to ice-water and extracted twice with ethyl acetate. The combined organic phases were washed with saturated sodium chloride solution and the solvent concentrated fully under reduced pressure. This gave 4.2 g of 4-[(4-methoxy-3-nitrophenyl)sulphonyl]tetrahydro-2H-pyran.

¹H-NMR (400 MHz, 25° C., DMSO-d6): δ=1.54 (qd, 2H); 1.70-1.78 (m, 2H); 3.27 (dt, 2H); 3.63 (tt, 1H); 3.87-3.95 (m, 2H); 4.05 (s, 3H); 7.63 (d, 1H); 8.09 (dd, 1H); 8.30 (d, 1H).

### Intermediate 38

### 2-Methoxy-5-(tetrahydro-2H-pyran-4-ylsulphonyl)aniline

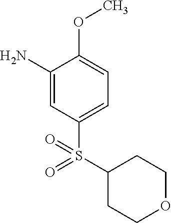 [SMILES: COc1ccc(S(=O)(=O)C2CCOCC2)cc1N]

A mixture of 4 g of intermediate 37 and 400 mg of palladium on carbon (10%) in 500 ml of methanol was shaken at RT under a 1 bar hydrogen atmosphere for 9 hours. The mixture was filtered through kieselguhr and the solution was fully concentrated. This gave 3.6 g of 2-methoxy-5-(tetrahydro-2H-pyran-4-ylsulphonyl)aniline

¹H-NMR (400 MHz, 25° C., DMSO-d6, signals sometimes obscured by water): δ=1.49 (qd, 2H); 1.66-1.75 (m, 2H); 3.26 (dt, 2H); 3.83-3.92 (m, 5H); 5.26 (s, 2H); 6.96-7.03 (m, 2H); 7.05 (d, 1H).

### Intermediate 39

### (3R)-6-Chloro-1,3-dimethyl-4-[1-(2,2,2-trifluoroethyl)piperidin-4-yl]-3,4-dihydropyrido[2,3-b]pyrazin-2(1H)-one

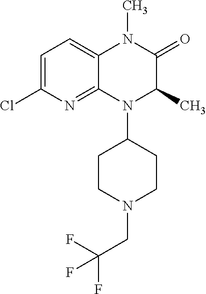 [SMILES: C[C@@H]1C(=O)N(C)c2ccc(Cl)nc2N1C1CCN(CC(F)(F)F)CC1]

A solution of 1.0 g of (3R)-6-chloro-1,3-dimethyl-4-(piperidin-4-yl)-3,4-dihydropyrido[2,3-b]pyrazin-2(1H)-one hydrochloride (CAS 1644414-06-1, preparation described in WO2014095774, intermediate 64) and 852 mg of 2,2,2-trifluoroethyl trifluoromethanesulphonate (CAS 6226-25-1) in 1.4 ml of triethylamine and 16 ml of THF was stirred at a bath temperature of 70° C. under an argon atmosphere for 14 hours. The mixture was concentrated under reduced pressure and the residue was purified by chromatography on silica gel (hexane/ethyl acetate 50:50). This gave 630 mg of (3R)-6-chloro-1,3-dimethyl-4-[1-(2,2,2-trifluoroethyl)piperidin-4-yl]-3,4-dihydropyrido[2,3-b]pyrazin-2(1H)-one.

¹H-NMR (400 MHz, 25° C., DMSO-d6, some signals overlayed by DMSO): δ=1.11 (d, 3H); 1.55 (bd, 1H); 1.74-1.90 (m, 2H); 1.97 (qd, 1H); 2.94-3.05 (m, 2H); 3.15-3.26 (s+q, 5H); 4.07 (tt, 1H); 4.30 (q, 1H); 6.79 (d, 1H); 7.32 (d, 1H).

Optical rotation [α]_{D}²⁰=−106.3°+/−0.33° (c=6.0 mg/ml, methanol).

### Intermediate 40

### (3R)-6-Chloro-4-[1-(2,2-difluoroethyl)piperidin-4-yl]-1,3-dimethyl-3,4-dihydropyrido[2,3-b]pyrazin-2(1H)-one

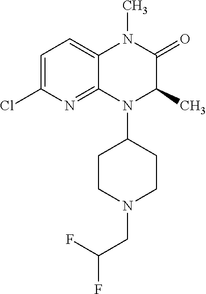 [SMILES: C[C@@H]1C(=O)N(C)c2ccc(Cl)nc2N1C1CCN(CC(F)F)CC1]

A solution of 1.0 g of (3R)-6-chloro-1,3-dimethyl-4-(piperidin-4-yl)-3,4-dihydropyrido[2,3-b]pyrazin-2(1H)-one hydrochloride (CAS 1644414-06-1, preparation described in WO2014095774, intermediate 64), 1.31 g of 2,2-difluoroethyl trifluoromethanesulphonate (CAS 74427-22-8), 1.01 g of potassium carbonate and 406 mg of potassium iodide in 12.9 ml of acetonitrile was stirred at a bath temperature of 60° C. under an argon atmosphere for 14 hours. The mixture was filtered, concentrated under reduced pressure and the residue was purified by chromatography on silica gel (hexane/ethyl acetate gradient to 100% ethyl acetate content). This gave 536 mg of (3R)-6-chloro-4-[1-(2,2-difluoroethyl)piperidin-4-yl]-1,3-dimethyl-3,4-dihydropyrido[2,3-b]pyrazin-2(1H)-one.

¹H-NMR (400 MHz, 25° C., DMSO-d6): δ=1.11 (d, 3H); 1.56 (bd, 1H); 1.79 (qd, 1H); 1.85-2.02 (m, 2H); 2.25-2.35 (m, 2H); 2.75 (dt, 2H); 2.94-3.05 (m, 2H); 3.22 (s, 3H); 4.06 (tt, 1H); 4.29 (q, 1H); 6.13 (tt, 1H); 6.79 (d, 1H); 7.32 (d, 1H).

Optical rotation [α]_{D}²⁰=−126.4°+/−0.19° (c=8.0 mg/ml, methanol).

### Intermediate 41

### (3R)-6-Chloro-1,3-dimethyl-4-[1-(3,3,3-trifluoropropyl)piperidin-4-yl]-3,4-dihydropyrido[2,3-b]pyrazin-2(1H)-one

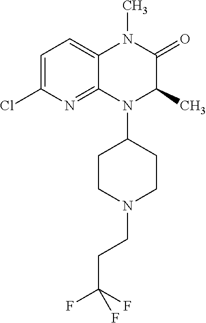 [SMILES: C[C@@H]1C(=O)N(C)c2ccc(Cl)nc2N1C1CCN(CCC(F)(F)F)CC1]

A solution of 1.5 g of (3R)-6-chloro-1,3-dimethyl-4-(piperidin-4-yl)-3,4-dihydropyrido[2,3-b]pyrazin-2(1H)-one hydrochloride (CAS 1644414-06-1, preparation described in WO2014095774, intermediate 64), 0.88 g of 1-iodo-3,3,3-trifluoropropane (CAS 460-37-7) and 1.76 g of potassium carbonate in 10 ml of acetonitrile was stirred at a bath temperature of 70° C. under an argon atmosphere for 4 hours and at RT for 14 hours. The mixture was added to water and extracted with dichloromethane. The organic phase was washed with water and saturated sodium chloride solution, dried over sodium sulphate, and the solvent was removed under reduced pressure. The residue was purified by chromatography on silica gel (dichloromethane/methanol gradient up to 10% methanol content). This gave 620 mg of (3R)-6-chloro-1,3-dimethyl-4-[1-(3,3,3-trifluoropropyl)piperidin-4-yl]-3,4-dihydropyrido[2,3-b]pyrazin-2(1H)-one.

¹H-NMR (400 MHz, 25° C., DMSO-d6): δ=1.11 (d, 3H); 1.56 (bd, 1H); 1.79 (qd, 1H); 1.85-2.02 (m, 2H); 2.25-2.35 (m, 2H); 2.75 (dt, 2H); 2.94-3.05 (m, 2H); 3.22 (s, 3H); 4.06 (tt, 1H); 4.29 (q, 1H); 6.13 (tt, 1H); 6.79 (d, 1H); 7.32 (d, 1H).

### Intermediate 42

### Ethyl 4-(4-fluorophenoxy)-3-nitrophenyl sulphone

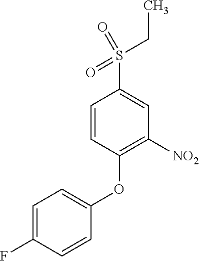 [SMILES: CCS(=O)(=O)c1ccc(Oc2ccc(F)cc2)c([N+](=O)[O-])c1]

A mixture of 2 g of 1-chloro-4-(ethylsulphonyl)-2-nitrobenzene (CAS 74159-80-1), 898 mg of 4-fluorophenol (CAS 371-41-5) and 1.22 g of potassium carbonate in 40 ml of DMF was stirred for 4 hours at a bath temperature of 70° C. The mixture was added to water and extracted twice with ethyl acetate. The organic phase was washed three times with semi-saturated aqueous sodium chloride solution, dried over sodium sulphate and the solvent was removed under reduced pressure. The residue was purified by chromatography on silica gel (hexane/ethyl acetate gradient up to 50% ethyl acetate content). This gave 2.4 g of ethyl 4-(4-fluorophenoxy)-3-nitrophenyl sulphone.

¹H-NMR (400 MHz, 25° C., DMSO-d6): δ=1.13 (t, 3H); 3.40 (q, 2H); 7.19 (d, 1H); 7.31-7.40 (m, 4H); 8.08 (dd, 1H); 8.51 (d, 1H).

### Intermediate 43

### 5-(Ethylsulphonyl)-2-(4-fluorophenoxy)aniline

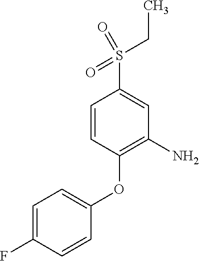 [SMILES: CCS(=O)(=O)c1ccc(Oc2ccc(F)cc2)c(N)c1]

A mixture of 2 g of intermediate 42 and 214 mg of palladium on carbon (10%) in 41 ml of methanol was shaken at RT under a 1 bar hydrogen atmosphere. After uptake of ca. 120 ml of hydrogen, the mixture was filtered through silica gel and the solution fully concentrated. This gave 1.96 g of 5-(ethylsulphonyl)-2-(4-fluorophenoxy)aniline.

¹H-NMR (400 MHz, 25° C., DMSO-d6): δ=1.10 (t, 3H); 3.17 (q, 2H); 5.61 (bs, 2H); 6.80 (d, 1H); 6.96 (dd, 1H); 7.06-7.11 (m, 2H); 7.21-7.28 (m, 3H).

### Intermediate 44

### Ethyl 3-nitro-4-phenoxyphenyl sulphone

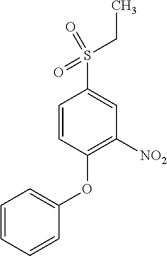 [SMILES: CCS(=O)(=O)c1ccc(Oc2ccccc2)c([N+](=O)[O-])c1]

A mixture of 1 g of 1-chloro-4-(ethylsulphonyl)-2-nitrobenzene (CAS 74159-80-1), 377 mg of phenol and 609 mg of potassium carbonate in 20 ml of DMF was stirred for 4 hours at a bath temperature of 70° C. The mixture was added to water and extracted twice with ethyl acetate. The organic phase was washed three times with semi-saturated aqueous sodium chloride solution, dried over sodium sulphate and the solvent was removed under reduced pressure. The residue was purified by chromatography on silica gel (hexane/ethyl acetate gradient up to 50% ethyl acetate content). This gave 1.15 g of ethyl 3-nitro-4-phenoxyphenyl sulphone.

¹H-NMR (400 MHz, 25° C., DMSO-d6): δ=1.13 (t, 3H); 3.40 (q, 2H); 7.19 (d, 1H); 7.26 (d, 2H); 7.35 (t, 1H); 7.52 (dd, 2H); 8.10 (dd, 1H); 8.52 (d, 1H).

### Intermediate 45

### 5-(Ethylsulphonyl)-2-phenoxyaniline

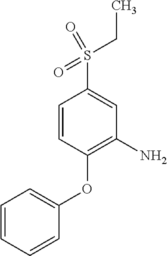 [SMILES: CCS(=O)(=O)c1ccc(Oc2ccccc2)c(N)c1]

A mixture of 1.1 g of intermediate 44 and 124 mg of palladium on carbon (10%) in 24 ml of methanol was shaken at RT under a 1 bar hydrogen atmosphere. After uptake of ca. 190 ml of hydrogen, the mixture was filtered through kieselguhr and the solution fully concentrated. This gave 1.0 g of 5-(ethylsulphonyl)-2-phenoxyaniline.

¹H-NMR (400 MHz, 25° C., DMSO-d6): δ=1.10 (t, 3H); 3.17 (q, 2H); 5.58 (bs, 2H); 6.82 (d, 1H); 6.97 (dd, 1H); 7.03 (d, 2H); 7.16 (t, 1H); 7.26 (d, 1H); 7.41 (dd, 2H).

### Intermediate 46

### 1-(Cyclobutylsulphanyl)-4-methoxybenzene

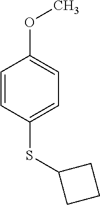

A mixture of 2.8 g of 4-methoxythiophenol (CAS 696-63-9) and 5.12 g of bromocyclobutane (CAS 25637-16-5) and 5.3 ml of sodium methoxide solution (30% in methanol) in 13.7 ml of methanol was stirred for 4 hours at a bath temperature of 60° C. The mixture was added to water and extracted three times with ethyl acetate. The combined organic phases were washed with semi-saturated sodium chloride solution, dried over sodium sulphate and the solvent was removed completely under reduced pressure. The residue was purified by chromatography on silica gel (hexane/ethyl acetate gradient up to 5% ethyl acetate content). This gave 1.1 g of 1-(cyclobutylsulphanyl)-4-methoxybenzene.

¹H-NMR: (400 MHz, 25° C., DMSO-d6): δ=1.82-1.97 (m, 4H); 2.25-2.35 (m, 2H); 3.73-3.82 (m+s, 4H); 6.91 (d, 2H); 7.25 (d, 2H).

### Intermediate 47

### Cyclobutyl 4-methoxyphenyl sulphone

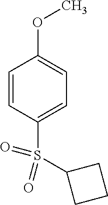

A total of 3.31 g of potassium peroxymonosulphate (Oxone®, CAS 70693-62-8) were added portionwise at RT to a solution of 1.1 g of intermediate 46 in 38 ml of methanol and the mixture was then stirred at RT for 14 hours. The mixture was concentrated under reduced pressure and the residue was taken up in dichloromethane. The mixture was washed with 1 N hydrochloric acid and saturated aqueous sodium chloride solution, dried over sodium sulphate, and the solvent was removed completely under reduced pressure. The residue was purified by chromatography on silica gel (hexane/ethyl acetate gradient up to 10% ethyl acetate content). This gave 1.0 g of cyclobutyl 4-methoxyphenyl sulphone.

¹H-NMR (400 MHz, 25° C., DMSO-d6): δ=1.77-1.97 (m, 2H); 2.04-2.14 (m, 2H); 2.23-2.35 (m, 2H); 3.85 (s, 3H); 4.02 (qi, 1H); 7.16 (d, 2H); 7.77 (d, 2H).

### Intermediate 48

### Cyclobutyl 4-methoxy-3-nitrophenyl sulphone

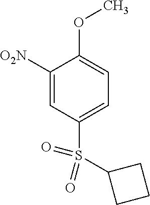 [SMILES: COc1ccc(S(=O)(=O)C2CCC2)cc1[N+](=O)[O-]]

0.26 ml of concentrated nitric acid (65%) was added dropwise at 0° C. to a mixture of 800 mg of intermediate 47 in 3.71 ml of concentrated sulphuric acid. The mixture was stirred at 0° C. for 10 min. The mixture was added to ice-water and extracted twice with ethyl acetate. The combined organic phases were washed with saturated sodium chloride solution and the solvent concentrated fully under reduced pressure. The residue was purified by chromatography on silica gel (hexane/ethyl acetate gradient up to 20% ethyl acetate content). This gave 700 mg of cyclobutyl 4-methoxy-3-nitrophenyl sulphone.

¹H-NMR (400 MHz, 25° C., DMSO-d6): δ=1.81-2.00 (m, 2H); 2.08-2.18 (m, 2H); 2.27-2.39 (m, 2H); 4.03 (s, 3H); 4.19 (qi, 1H); 7.60 (d, 1H); 8.11 (dd, 1H); 8.32 (d, 1H).

### Intermediate 49

### 5-(Cyclobutylsulphonyl)-2-methoxyaniline

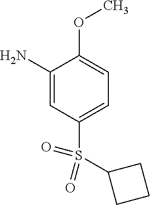 [SMILES: COc1ccc(S(=O)(=O)C2CCC2)cc1N]

A mixture of 700 mg of intermediate 48 and 80 mg of palladium on carbon (10%) in 51 ml of methanol and 51 ml of ethyl acetate was shaken at RT under a 1 bar hydrogen atmosphere for 7 hours. The mixture was filtered through kieselguhr and the solution was fully concentrated. This gave 610 mg of 5-(cyclobutylsulphonyl)-2-methoxyaniline.

¹H-NMR (400 MHz, 25° C., DMSO-d6): δ=1.77-1.96 (m, 2H); 2.04-2.14 (m, 2H); 2.21-2.34 (m, 2H); 3.84 (s, 3H); 3.89 (qi, 1H); 5.23 (bs, 2H); 6.95-7.01 (m, 2H); 7.06 (d, 1H).

### Preparation of the Inventive Compounds

The examples below describe the preparation of the compounds according to the invention.

### Example 1

### (3R)-1,3-Dimethyl-6-{[3-(methylsulphonyl)phenyl]amino}-4-(tetrahydro-2H-pyran-4-yl)-3,4-dihydropyrido[2,3-b]pyrazin-2(1H)-one

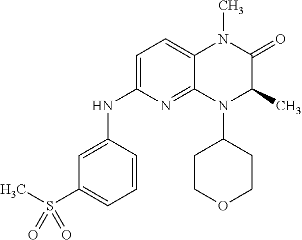

A mixture of 150 mg of intermediate 4, 150 mg of 3-(methylsulphonyl)aniline hydrochloride (CAS 80213-28-1), 6.6 mg of tris(dibenzylideneacetone)dipalladium(0) (CAS 51364-51-3), 235 mg of caesium carbonate and 12 mg of Xanthphos (CAS 161265-03-8) in 15 ml of dioxane was stirred under an argon atmosphere at 120° C. for 44 hours. The mixture was added to water and extracted twice with ethyl acetate. The organic phase was washed with saturated aqueous sodium chloride solution, dried over sodium sulphate and the solvent was removed under reduced pressure. The residue was purified by RP-HPLC chromatography (column: X-Bridge C18 5 μm 100×30 mm, mobile phase: acetonitrile/water (0.1% by volume formic acid) gradient). This gave 75 mg of (3R)-1,3-dimethyl-6-{[3-(methylsulphonyl)phenyl]amino}-4-(tetrahydro-2H-pyran-4-yl)-3,4-dihydropyrido[2,3-b]pyrazin-2(1H)-one.

¹H-NMR: (400 MHz, 25° C., DMSO-d6): δ=1.08 (d, 3H); 1.54-1.62 (m, 1H); 1.76 (qd, 1H); 1.87 (qd, 1H); 1.90-1.98 (m, 1H); 3.18 (s, 3H); 3.21 (s, 3H); 3.48-3.58 (m, 2H); 3.91 (dt, 2H); 4.24 (q, 1H); 4.50 (tt, 1H); 6.29 (d, 1H); 7.30 (d, 1H); 7.33-7.37 (m, 1H); 7.49 (t, 1H); 7.92 (dd, 1H); 8.12 (t, 1H); 9.24 (s, 1H).

chiral HPLC: Rt=5.14 min, (97% ee)

Instrument: Waters Alliance 2695; column: Chiralpak IA 3 μm 100×4.6 mm; mobile phase: hexane/2-propanol 70:30; flow rate 1 ml/min; temperature: 25° C.; injection: 5 μl (1 mg/ml ethanol/methanol, 1:1); DAD 996 scan: 280 nm.

### Example 2

### (3R)-1,3-Dimethyl-6-{[2-methyl-5-(methylsulphonyl)phenyl]amino}-4-(tetrahydro-2H-pyran-4-yl)-3,4-dihydropyrido[2,3-b]pyrazin-2(1H)-one

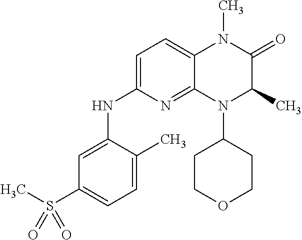

A mixture of 150 mg of intermediate 4, 233 mg of 2-methyl-5-(methylsulphonyl)aniline (CAS 1671-48-3), 6.6 mg of tris(dibenzylideneacetone)dipalladium(0) (CAS 51364-51-3), 235 mg of caesium carbonate and 12 mg of Xanthphos (CAS 161265-03-8) in 15 ml of dioxane was stirred under an argon atmosphere at 120° C. for 26 hours and then at RT for 10 hours. The mixture was added to water and extracted twice with ethyl acetate. The organic phase was washed with saturated aqueous sodium chloride solution, dried over sodium sulphate and the solvent was removed under reduced pressure. The residue was purified by RP-HPLC chromatography (column: X-Bridge C18 5 μm 100×30 mm, mobile phase: acetonitrile/water (0.1% by volume formic acid) gradient). This gave 130 mg of (3R)-1,3-dimethyl-6-{[2-methyl-5-(methylsulphonyl)phenyl]amino}-4-(tetrahydro-2H-pyran-4-yl)-3,4-dihydropyrido[2,3-b]pyrazin-2(1H)-one.

¹H-NMR: (400 MHz, 25° C., DMSO-d6): δ=1.06 (d, 3H); 1.43-1.52 (m, 1H); 1.54-1.88 (m, 3H); 2.34 (s, 3H); 3.13 (s, 3H); 3.21 (s, 3H); 3.25-3-35 (m, 2H, signal sometimes masked by water peak); 3.45-3.56 (m, 1H); 3.72-3.87 (m, 2H); 4.20 (q, 1H); 4.42 (tt, 1H); 6.44 (d, 1H); 7.30 (d, 1H); 7.34-7.43 (m, 2H); 8.04 (s, 1H); 8.33 (d, 1H).

### Example 3

### (3R)-6-{[2-Methoxy-5-(methylsulphonyl)phenyl]amino}-1,3-dimethyl-4-(tetrahydro-2H-pyran-4-yl)-3,4-dihydropyrido[2,3-b]pyrazin-2(1H)-one

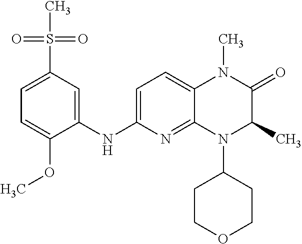 [SMILES: COc1ccc(S(C)(=O)=O)cc1Nc1ccc2c(n1)N(C1CCOCC1)[C@H](C)C(=O)N2C]

A mixture of 150 mg of intermediate 4, 145 mg of 2-methoxy-5-methylsulphonylaniline (CAS 20945-70-4), 6.6 mg of tris(dibenzylideneacetone)dipalladium(0) (CAS 51364-51-3), 235 mg of caesium carbonate and 12 mg of Xanthphos (CAS 161265-03-8) in 15 ml of dioxane was stirred under an argon atmosphere at 120° C. for 20 hours and then at RT for 10 hours. The mixture was added to water and extracted twice with ethyl acetate. The organic phase was washed with saturated aqueous sodium chloride solution, dried over sodium sulphate and the solvent was removed under reduced pressure. The residue was purified by chromatography on silica gel (dichloromethane/methanol gradient up to 3% methanol content). This gave 155 mg of (3R)-6-{[2-methoxy-5-(methylsulphonyl)phenyl]amino}-1,3-dimethyl-4-(tetrahydro-2H-pyran-4-yl)-3,4-dihydropyrido[2,3-b]pyrazin-2(1H)-one.

¹H-NMR: (400 MHz, 25° C., DMSO-d6): δ=1.06 (d, 3H); 1.48-1.55 (m, 1H); 1.68 (qd, 1H); 1.78 (qd, 1H); 1.84-1.91 (m, 1H); 3.13 (s, 1H); 3.21 (s, 3H); 3.48-3.62 (m, 2H); 3.77-3.86 (m, 2H); 4.21 (q, 1H); 4.56 (tt, 1H); 6.59 (d, 1H); 7.19 (d, 1H); 7.30 (d, 1H); 7.41 (dd, 1H); 8.17 (s, 1H); 8.67 (d, 1H).

### Example 4

### (3R)-6-[(1,1-Dioxido-2,3-dihydro-1-benzothiophen-6-yl)amino]-1,3-dimethyl-4-(tetrahydro-2H-pyran-4-yl)-3,4-dihydropyrido[2,3-b]pyrazin-2(1H)-one

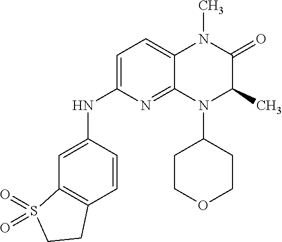 [SMILES: C[C@@H]1C(=O)N(C)c2ccc(Nc3ccc4c(c3)S(=O)(=O)CC4)nc2N1C1CCOCC1]

A mixture of 150 mg of intermediate 4, 132 mg of 2,3-dihydro-1-benzothiophen-6-amine 1,1-dioxide (CAS 20503-39-3), 6.6 mg of tris(dibenzylideneacetone)dipalladium(0) (CAS 51364-51-3), 235 mg of caesium carbonate and 12 mg of Xanthphos (CAS 161265-03-8) in 10 ml of dioxane was stirred under an argon atmosphere at 120° C. for 20 hours. The mixture was added to water and extracted twice with ethyl acetate. The organic phase was washed with saturated aqueous sodium chloride solution, dried over sodium sulphate and the solvent was removed under reduced pressure. The residue was purified by RP-HPLC chromatography (column: X-Bridge C18 5 μm 100×30 mm, mobile phase: acetonitrile/water (0.2% by volume ammonia (32%) gradient). This gave 50 mg of (3R)-6-[(1,1-dioxido-2,3-dihydro-1-benzothiophen-6-yl)amino]-1,3-dimethyl-4-(tetrahydro-2H-pyran-4-yl)-3,4-dihydropyrido[2,3-b]pyrazin-2(1H)-one.

¹H-NMR: (400 MHz, 25° C., DMSO-d6): δ=1.08 (d, 3H); 1.51-1.61 (m, 1H); 1.69-1.96 (m, 3H); 3.17-3.28 (m+s, 5H); 3.47-3.60 (m, 3H); 3.65 (dt, 1H), 3.85-3.97 (m, 2H); 4.25 (q, 1H); 4.52 (tt, 1H); 6.27 (d, 1H); 7.30 (d, 1H); 7.36 (d, 1H); 7.57 (dd, 1H); 8.27 (d, 1H); 9.25 (s, 1H).

### Example 5

### (3R)-4-Cycloheptyl-1,3-dimethyl-6-{[3-(methylsulphonyl)phenyl]amino}-3,4-dihydropyrido[2,3-b]pyrazin-2(1H)-one

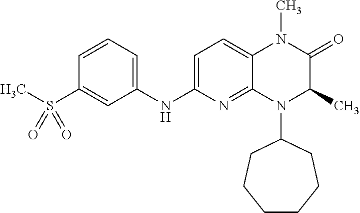 [SMILES: C[C@@H]1C(=O)N(C)c2ccc(Nc3cccc(S(C)(=O)=O)c3)nc2N1C1CCCCCC1]

A mixture of 150 mg of intermediate 10, 118 mg of 3-(methylsulphonyl)aniline hydrochloride (CAS 80213-28-1), 21.2 mg of tris(dibenzylideneacetone)dipalladium(0) (CAS 51364-51-3), 226 mg of caesium carbonate and 26.8 mg of Xanthphos (CAS 161265-03-8) in 4 ml of dioxane was stirred under an argon atmosphere at 120° C. for 8 hours. A further 21.2 mg of tris(dibenzylideneacetone)dipalladium(0) and 26.8 mg of Xanthphos were added and the mixture was stirred a further 8 hours under an argon atmosphere at 120° C. The mixture was added to water and extracted twice with ethyl acetate. The organic phase was washed with saturated aqueous sodium chloride solution, dried over sodium sulphate and the solvent was removed under reduced pressure. The residue was purified by RP-HPLC chromatography (column: X-Bridge C18 5 μm 100×30 mm, mobile phase: acetonitrile/water (0.1% by volume formic acid) gradient). This gave 110 mg of (3R)-4-cycloheptyl-1,3-dimethyl-6-{[3-(methylsulphonyl)phenyl]amino}-3,4-dihydropyrido[2,3-b]pyrazin2(1H)-one.

¹H-NMR: (400 MHz, 25° C., DMSO-d6): δ=1.08 (d, 3H); 1.44-1.76 (m, 11H); 1.77-1.90 (m, 1H); 1.99-2.10 (m, 1H); 3.16 (s, 3H); 3.20 (s, 3H); 4.24 (q, 1H); 4.37 (tt, 1H); 6.25 (d, 1H); 7.27 (d, 1H); 7.30-7.42 (m, 4H); 7.46 (t, 1H); 7.93 (t, 1H); 8.12 (dd, 1H).

### Example 6

### (3R)-6-{[3-(Cyclopropylsulphonyl)phenyl]amino}-1,3-dimethyl-4-(tetrahydro-2H-pyran-4-yl)-3,4-dihydropyrido[2,3-b]pyrazin-2(1H)-one

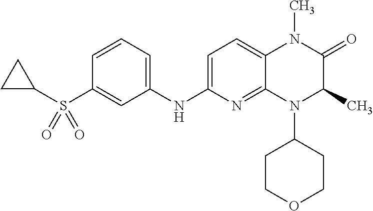 [SMILES: C[C@@H]1C(=O)N(C)c2ccc(Nc3cccc(S(=O)(=O)C4CC4)c3)nc2N1C1CCOCC1]

A mixture of 100 mg of intermediate 4, 95 mg of intermediate 22, 14.7 mg of tris(dibenzylideneacetone)dipalladium(0) (CAS 51364-51-3), 157 mg of caesium carbonate and 18.6 mg of Xanthphos (CAS 161265-03-8) in 2.7 ml of dioxane was stirred under an argon atmosphere at 120° C. for 8 hours. A further 14.7 mg of tris(dibenzylideneacetone)dipalladium(0) and 18.6 mg of Xanthphos were added and the mixture was stirred a further 8 hours under an argon atmosphere at 120° C. The mixture was added to water and extracted twice with ethyl acetate. The organic phase was washed with saturated aqueous sodium chloride solution, dried over sodium sulphate and the solvent was removed under reduced pressure. The residue was purified by RP-HPLC chromatography (column: X-Bridge C18 5 μm 100×30 mm, mobile phase: acetonitrile/water (0.1% by volume formic acid) gradient). This gave 100 mg of (3R)-6-{[3-(cyclopropylsulphonyl)phenyl]amino}-1,3-dimethyl-4-(tetrahydro-2H-pyran-4-yl)-3,4-dihydropyrido[2,3-b]pyrazin-2(1H)-one.

¹H-NMR: (400 MHz, 25° C., DMSO-d6): δ=0.99-1.13 (m, 7H); 1.57 (bd, 1H); 1.68-1.99 (m, 3H); 2.75-2.86 (m, 1H); 3.21 (s, 3H); 3.52 (bt, 2H); 3.85-3.99 (m, 2H); 4.24 (q, 1H); 4.48 (tt, 1H); 6.28 (d, 1H); 7.30 (d, 2H); 7.49 (t, 1H); 7.99 (d, 1H); 8.04 (t, 1H); 9.26 (s, 1H).

### Example 7

### (3R)-6-{[3-(Isopropylsulphonyl)phenyl]amino}-1,3-dimethyl-4-(tetrahydro-2H-pyran-4-yl)-3,4-dihydropyrido[2,3-b]pyrazin-2(1H)-one

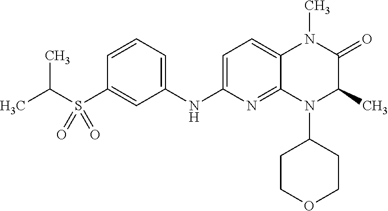 [SMILES: CC(C)S(=O)(=O)c1cccc(Nc2ccc3c(n2)N(C2CCOCC2)[C@H](C)C(=O)N3C)c1]

A mixture of 100 mg of intermediate 4, 96 mg of 3-(propan-2-ylsulphonyl)aniline (CAS 170856-37-8), 14.7 mg of tris(dibenzylideneacetone)dipalladium(0) (CAS 51364-51-3), 157 mg of caesium carbonate and 18.6 mg of Xanthphos (CAS 161265-03-8) in 2.7 ml of dioxane was stirred under an argon atmosphere at 120° C. for 8 hours. A further 14.7 mg of tris(dibenzylideneacetone)dipalladium(0) and 18.6 mg of Xanthphos were added and the mixture was stirred a further 8 hours under an argon atmosphere at 120° C. The mixture was added to water and extracted twice with ethyl acetate. The organic phase was washed with saturated aqueous sodium chloride solution, dried over sodium sulphate and the solvent was removed under reduced pressure. The residue was purified by RP-HPLC chromatography (column: X-Bridge C18 5 μm 100×30 mm, mobile phase: acetonitrile/water (0.2% by volume ammonia (32%) gradient). This gave 14 mg of (3R)-6-{[3-(isopropylsulphonyl)phenyl]amino}-1,3-dimethyl-4-(tetrahydro-2H-pyran-4-yl)-3,4-dihydropyrido[2,3-b]pyrazin-2(1H)-one.

¹H-NMR: (400 MHz, 25° C., DMSO-d6): δ=1.08 (d, 3H); 1.16 (d, 6H); 1.58 (bd, 1H); 1.77 (qd, 1H); 1.82-1.98 (m, 2H); 3.21 (s, 3H); 3.36 (sept, 1H, signal sometimes under water peak); 3.48-3.59 (m, 2H); 3.88-3.99 (m, 2H); 4.24 (q, 1H); 4.49 (tt, 1H); 6.29 (d, 1H); 7.26 (d, 1H); 7.30 (d, 1H); 7.50 (t, 1H); 7.99 (t, 1H); 8.04 (d, 1H); 9.24 (s, 1H).

### Example 8

### (3R)-6-{[5-(Cyclopropylsulphonyl)-2-methoxyphenyl]amino}-1,3-dimethyl-4-(tetrahydro-2H-pyran-4-yl)-3,4-dihydropyrido[2,3-b]pyrazin-2(1H)-one

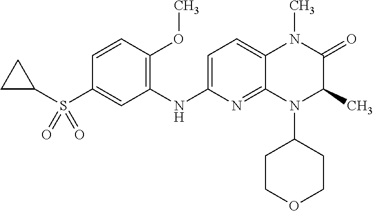 [SMILES: COc1ccc(S(=O)(=O)C2CC2)cc1Nc1ccc2c(n1)N(C1CCOCC1)[C@H](C)C(=O)N2C]

A mixture of 150 mg of intermediate 4, 164 mg of intermediate 26, 22.1 mg of tris(dibenzylideneacetone)dipalladium(0) (CAS 51364-51-3), 235 mg of caesium carbonate and 22.1 mg of Xanthphos (CAS 161265-03-8) in 12.9 ml of dioxane was stirred under an argon atmosphere at 120° C. for 20 hours. The mixture was added to water and extracted twice with ethyl acetate. The organic phase was washed with saturated aqueous sodium chloride solution, dried over sodium sulphate and the solvent was removed under reduced pressure. The residue was purified by chromatography on silica gel (dichloromethane/methanol gradient up to 3% methanol content). This gave 125 mg of (3R)-6-{[5-(cyclopropylsulphonyl)-2-methoxyphenyl]amino}-1,3-dimethyl-4-(tetrahydro-2H-pyran-4-yl)-3,4-dihydropyrido[2,3-b]pyrazin-2(1H)-one.

¹H-NMR: (400 MHz, 25° C., DMSO-d6): δ=0.95-1.10 (m, 7H); 1.52 (bd, 1H); 1.69 (qd, 1H); 1.77 (qd, 1H); 1.86 (bd, 1H); 2.69-2.78 (m, 1H); 3.21 (s, 3H); 3.50-3.60 (m, 2H); 3.77-3.86 (m, 2H); 3.96 (s, 3H); 4.21 (q, 1H); 6.58 (d, 1H); 7.19 (d, 1H); 7.30 (d, 1H); 7.37 (dd, 1H); 8.14 (s, 1H); 8.63 (d, 1H).

### Example 9

(3R)-6-{[5-(Isopropylsulphonyl)-2-methoxyphenyl]amino}-1,3-dimethyl-4-(tetrahydro-2H-pyran-4-yl)-3,4-dihydropyrido[2,3-b]pyrazin-2(1H)-one

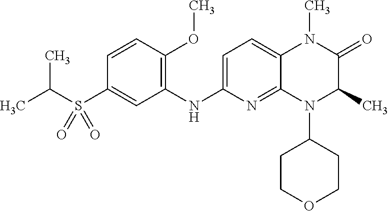 [SMILES: COc1ccc(S(=O)(=O)C(C)C)cc1Nc1ccc2c(n1)N(C1CCOCC1)[C@H](C)C(=O)N2C]

A mixture of 150 mg of intermediate 4, 165 mg of intermediate 30, 22.1 mg of tris(dibenzylideneacetone)dipalladium(0) (CAS 51364-51-3), 235 mg of caesium carbonate and 27.9 mg of Xanthphos (CAS 161265-03-8) in 12.9 ml of dioxane was stirred under an argon atmosphere at 120° C. for 20 hours. The mixture was added to water and extracted twice with ethyl acetate. The organic phase was washed with saturated aqueous sodium chloride solution, dried over sodium sulphate and the solvent was removed under reduced pressure. The residue was purified by chromatography on silica gel (dichloromethane/methanol gradient up to 3% methanol content). The resulting product was triturated with methyl tert-butyl ether and filtered off with suction. This gave 170 mg of (3R)-6-{[5-(isopropylsulphonyl)-2-methoxyphenyl]amino}-1,3-dimethyl-4-(tetrahydro-2H-pyran-4-yl)-3,4-dihydropyrido[2,3-b]pyrazin-2(1H)-one.

¹H-NMR: (400 MHz, 25° C., DMSO-d6): δ=1.06 (d, 3H); 1.13 (d, 6H); 1.51 (bd, 1H); 1.68 (qd, 1H); 1.76 (qd, 1H); 1.85 (bd, 1H); 3.21 (s, 3H); 3.31 (sept, 1H, signal sometimes under water peak); 3.52-3.62 (m, 2H); 3.77.3.87 (m, 2H); 3.97 (s, 3H); 4.21 (q, 1H); 4.55 (tt, 1H); 6.57 (d, 1H); 7.21 (d, 1H); 7.30 (d, 1H); 7.34 (dd, 1H); 8.14 (s, 1H); 8.57 (d, 1H).

### Example 10

### Ethyl [(3-{[(3R)-1,3-dimethyl-2-oxo-4-(tetrahydro-2H-pyran-4-yl)-1,2,3,4-tetrahydropyrido[2,3-b]pyrazin-6-yl]amino}phenyl)(methyl)oxido-λ⁶-sulphanylidene]carbamate

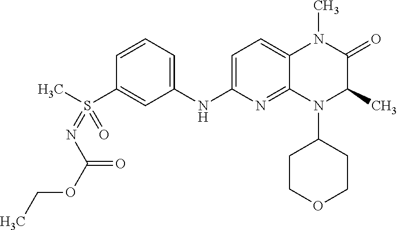 [SMILES: CCOC(=O)N=S(C)(=O)c1cccc(Nc2ccc3c(n2)N(C2CCOCC2)[C@H](C)C(=O)N3C)c1]

A mixture of 100 mg of intermediate 4, 123 mg of ethyl [(3-aminophenyl)(methyl)oxido-λ⁶-sulphanylidene]carbamate (preparation described in WO2007071455 and WO2008006560), 9.3 mg of tris(dibenzylideneacetone)dipalladium(0) (CAS 51364-51-3), 39 mg of sodium tert-butoxide and 13.3 mg of 2′-(dicyclohexylphosphanyl)-N,N-dimethylbiphenyl-2-amine (CAS 213697-53-1) in 4 ml of THF was stirred at 75° C. for 2 hours under an argon atmosphere. The mixture was evaporated to dryness and the residue was purified by RP-HPLC chromatography (column: X-Bridge C18 5 μm 100×30 mm, mobile phase: acetonitrile/water (0.2% by volume ammonia (32%) gradient). This gave 27 mg of ethyl [(3-{[(3R)-1,3-dimethyl-2-oxo-4-(tetrahydro-2H-pyran-4-yl)-1,2,3,4-tetrahydropyrido[2,3-b]pyrazin-6-yl]amino}phenyl)(methyl)oxido-λ⁶-sulphanylidene]carbamate as a mixture of diastereomers.

¹H-NMR: (400 MHz, 25° C., DMSO-d6): δ=1.05-1.14 (m, 6H); 1.58 (bd, 1H); 1.71-2.01 (m, 3H); 3.22 (s, 3H); 3.42 (2*s, 3H); 3.45-4.58 (m, 2H); 3.85-3.99 (m, 4H); 4.24 (q, 1H); 4.49 (tt, 1H); 6.30 (d, 1H); 7.30 (d, 1H); 7.34 bd, 1H); 7.51 (t, 1H); 8.02-8.11 (m, 2H); 9.30 (s, 1H).

### Example 11

### (3R)-1,3-Dimethyl-4-(1-methylpiperidin-4-yl)-6-{[3-(methylsulphonyl)phenyl]amino}-3,4-dihydropyrido[2,3-b]pyrazin-2(1H)-one

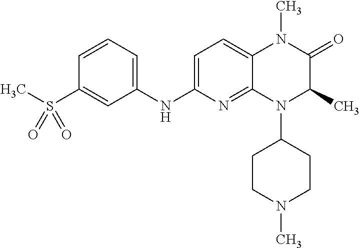

A mixture of 150 mg of intermediate 15, 118 mg of 3-(methylsulphonyl)aniline hydrochloride (CAS 80213-28-1), 21.1 mg of tris(dibenzylideneacetone)dipalladium(0) (CAS 51364-51-3), 226 mg of caesium carbonate and 26.7 mg of Xanthphos (CAS 161265-03-8) in 7 ml of dioxane was stirred under an argon atmosphere at 120° C. for 14 hours. The mixture was added to water and extracted twice with ethyl acetate. The organic phase was washed with saturated aqueous sodium chloride solution, dried over sodium sulphate and the solvent was removed under reduced pressure. The residue was purified by chromatography on silica gel (dichloromethane/methanol gradient up to 5% methanol content). This gave 11 mg of (3R)-1,3-dimethyl-4-(1-methylpiperidin-4-yl)-6-{[3-(methylsulphonyl)phenyl]amino}-3,4-dihydropyrido[2,3-b]pyrazin-2(1H)-one.

¹H-NMR: (400 MHz, 25° C., DMSO-d6): δ=1.09 (d, 3H); 1.57 (bd, 1H); 1.72 (qd, 1H); 1.86 (qd, 1H); 1.97 (bd, 1H); 2.04-2.14 (m, 2H); 2.78-2.89 (m, 2H); 3.17 (s, 3H); 3.21 (s, 3H); 4.16-4.25 (m, 2H); 6.27 (d, 1H); 7.29 (d, 1H); 7.34 (bd, 1H); 7.47 (t, 1H); 7.94 (t, 1H); 8.11 (bd, 1H); 9.23 (s, 1H).

### Example 12

### (3R)-6-{[5-(Cyclopropylsulphonyl)-2-methoxyphenyl]amino}-1,3-dimethyl-4-(1-methylpiperidin-4-yl)-3,4-dihydropyrido[2,3-b]pyrazin-2(1H)-one

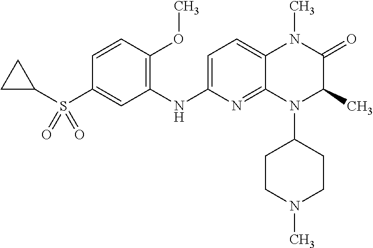 [SMILES: COc1ccc(S(=O)(=O)C2CC2)cc1Nc1ccc2c(n1)N(C1CCN(C)CC1)[C@H](C)C(=O)N2C]

A mixture of 100 mg of intermediate 15, 105 mg of intermediate 26, 14.1 mg of tris(dibenzylideneacetone)dipalladium(0) (CAS 51364-51-3), 150 mg of caesium carbonate and 17.8 mg of Xanthphos (CAS 161265-03-8) in 8.2 ml of dioxane was stirred under an argon atmosphere at 120° C. for 20 hours. A further 14.1 mg of tris(dibenzylideneacetone)dipalladium(0) (CAS 51364-51-3), 150 mg of caesium carbonate, 12.1 g of (2′-aminobiphenyl-2-yl)(chloro)palladium-dicyclohexyl[2′,4′,6′-tri(propan-2-yl)biphenyl-2-yl]phosphane (1:1) (CAS 1310584-14-5, commercially available) and 17.8 mg of Xanthphos were added and the mixture was stirred for a further 8 hours at 120° C. under an argon atmosphere. The mixture was added to water and extracted twice with ethyl acetate. The organic phase was washed with saturated aqueous sodium chloride solution, dried over sodium sulphate and the solvent was removed under reduced pressure. The residue was firstly pre-purified by chromatography on silica gel (dichloromethane/methanol gradient up to 10% methanol content). The crude product was purified by RP-HPLC chromatography (column: X-Bridge C18 5 μm 100×30 mm, mobile phase: acetonitrile/water (0.1% by volume formic acid) gradient). This gave 13 mg of (3R)-6-{[5-(cyclopropylsulphonyl)-2-methoxyphenyl]amino}-1,3-dimethyl-4-(1-methylpiperidin-4-yl)-3,4-dihydropyrido[2,3-b]pyrazin-2(1H)-one.

¹H-NMR: (400 MHz, 25° C., DMSO-d6): δ=0.95-1.09 (m, 7H); 1.55 (bd, 1H); 1.69 (qd, 1H); 1.79 (qd, 1H); 1.90 (bd, 1H); 2.24-2.36 (m, 2H); 2.70-2.84 (m, 3H); 3.21 (s, 3H); 3.96 (s, 3H); 4.17 (q, 1H); 4.31 (tt, 1H); 6.57 (d, 1H); 7.19 (d, 1H); 7.29 (d, 1H); 7.36 (dd, 1H); 8.13 (s, 1H); 8.58 (d, 1H).

### Example 13

### (3R)-6-{[5-(Isopropylsulphonyl)-2-methoxyphenyl]amino}-1,3-dimethyl-4-(1-methylpiperidin-4-yl)-3,4-dihydropyrido[2,3-b]pyrazin-2(1H)-one

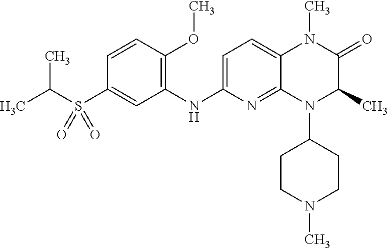 [SMILES: COc1ccc(S(=O)(=O)C(C)C)cc1Nc1ccc2c(n1)N(C1CCN(C)CC1)[C@H](C)C(=O)N2C]

A mixture of 100 mg of intermediate 15, 106 mg of intermediate 30, 14.1 mg of tris(dibenzylideneacetone)dipalladium(0) (CAS 51364-51-3), 150 mg of caesium carbonate and 17.8 mg of Xanthphos (CAS 161265-03-8) in 8.2 ml of dioxane was stirred under an argon atmosphere at 120° C. for 20 hours. The mixture was added to water and extracted twice with ethyl acetate. The organic phase was washed with saturated aqueous sodium chloride solution, dried over sodium sulphate and the solvent was removed under reduced pressure. The residue was purified by chromatography on silica gel (dichloromethane/methanol gradient up to 10% methanol content). This gave 10 mg of (3R)-6-{[5-(isopropylsulphonyl)-2-methoxyphenyl]amino}-1,3-dimethyl-4-(1-methylpiperidin-4-yl)-3,4-dihydropyrido[2,3-b]pyrazin-2(1H)-one.

¹H-NMR: (400 MHz, 25° C., DMSO-d6): δ=1.05 (d, 3H); 1.14 (d, 6H); 1.51 (bd, 1H); 1.65 (qd, 1H); 1.75 (qd, 1H); 1.87 (bd, 1H); 2.14-2.27 (m, 2H); 2.68-2.78 (m, 2H); 3.21 (s, 1H); 3.30 (sept, 1H signal sometimes under water peak; 3.96 (s, 3H); 4.16 (q, 1H); 4.28 (tt, 1H); 6.55 (d, 1H); 7.20 (d, 1H); 7.29 (d, 1H); 7.34 (dd, 1H); 8.09 (s, 1H); 8.49 (d, 1H).

### Example 14

### (3R)-4-Isopropyl-6-{[2-methoxy-5-(methylsulphonyl)phenyl]amino}-1,3-dimethyl-3,4-dihydropyrido[2,3-b]pyrazin-2(1H)-one

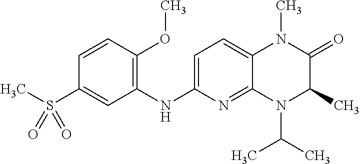

A mixture of 150 mg of intermediate 7, 170 mg of 2-methoxy-5-(methylsulphonyl)aniline (CAS 20945-70-4), 25.7 mg of tris(dibenzylideneacetone)dipalladium(0) (CAS 51364-51-3), 274 mg of caesium carbonate and 32.5 mg of Xanthphos (CAS 161265-03-8) in 15 ml of dioxane was stirred under an argon atmosphere at 120° C. for 20 hours. The mixture was added to water and extracted twice with ethyl acetate. The organic phase was washed with saturated aqueous sodium chloride solution, dried over sodium sulphate and the solvent was removed under reduced pressure. The residue was purified by RP-HPLC chromatography (column: X-Bridge C18 5 μm 100×30 mm, mobile phase: acetonitrile/water (0.1% by volume formic acid) gradient). This gave 145 mg of (3R)-4-isopropyl-6-{[2-methoxy-5-(methylsulphonyl)phenyl]amino}-1,3-dimethyl-3,4-dihydropyrido[2,3-b]pyrazin-2(1H)-one.

¹H-NMR: (400 MHz, 25° C., DMSO-d6): δ=1.09 (d, 3H); 1.21 (d, 3H); 1.29 (d, 3H); 3.09 (s, 3H); 3.21 (s, 3H); 3.97 (s, 3H); 4.24 (q, 1H); 4.78 (sept, 1H); 6.58 (d, 1H); 7.18 (d, 1H); 7.27 (d, 1H); 7.38 (dd, 1H); 8.18 (s, 1H); 8.99 (d, 1H).

### Example 15

### (3R)-6-{[5-(Cyclopropylsulphonyl)-2-methoxyphenyl]amino}-4-isopropyl-1,3-dimethyl-3,4-dihydropyrido[2,3-b]pyrazin-2(1H)-one

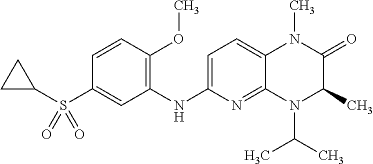 [SMILES: COc1ccc(S(=O)(=O)C2CC2)cc1Nc1ccc2c(n1)N(C(C)C)[C@H](C)C(=O)N2C]

A mixture of 100 mg of intermediate 7, 128 mg of intermediate 26, 17.1 mg of tris(dibenzylideneacetone)dipalladium(0) (CAS 51364-51-3), 183 mg of caesium carbonate and 21.7 mg of Xanthphos (CAS 161265-03-8) in 10 ml of dioxane was stirred under an argon atmosphere at 120° C. for 20 hours. The mixture was added to water and extracted twice with ethyl acetate. The organic phase was washed with saturated aqueous sodium chloride solution, dried over sodium sulphate and the solvent was removed under reduced pressure. The residue was purified by RP-HPLC chromatography (column: X-Bridge C18 5 μm 100×30 mm, mobile phase: acetonitrile/water (0.1% by volume formic acid) gradient). This gave 70 mg of (3R)-6-{[5-(cyclopropylsulphonyl)-2-methoxyphenyl]amino}-4-isopropyl-1,3-dimethyl-3,4-dihydropyrido[2,3-b]pyrazin-2(1H)-one

¹H-NMR: (400 MHz, 25° C., DMSO-d6): δ=0.96-1.06 (m, 4H); 1.09 (d, 3H); 1.20 (d, 3H); 1.28 (d, 3H); 2.63-2.71 (m, 1H); 3.21 (s, 3H); 3.97 (s, 3H); 4.24 (q, 1H); 4.80 (sept, 1H); 6.58 (d, 1H); 7.18 (d, 1H); 7.27 (d, 1H); 7.33 (dd, 1H); 8.16 (s, 1H); 8.95 (d, 1H).

### Example 16

### (3R)-4-Isopropyl-6-{[5-(isopropylsulphonyl)-2-methoxyphenyl]amino}-1,3-dimethyl-3,4-dihydropyrido[2,3-b]pyrazin-2(1H)-one

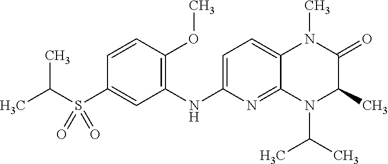 [SMILES: COc1ccc(S(=O)(=O)C(C)C)cc1Nc1ccc2c(n1)N(C(C)C)[C@H](C)C(=O)N2C]

A mixture of 100 mg of intermediate 7, 129 mg of intermediate 30, 17.1 mg of tris(dibenzylideneacetone)dipalladium(0) (CAS 51364-51-3), 183 mg of caesium carbonate and 21.7 mg of Xanthphos (CAS 161265-03-8) in 10 ml of dioxane was stirred under an argon atmosphere at 120° C. for 20 hours. The mixture was added to water and extracted twice with ethyl acetate. The organic phase was washed with saturated aqueous sodium chloride solution, dried over sodium sulphate and the solvent was removed under reduced pressure. The residue was purified by RP-HPLC chromatography (column: X-Bridge C18 5 μm 100×30 mm, mobile phase: acetonitrile/water (0.1% by volume formic acid) gradient). This gave 111 mg of (3R)-4-isopropyl-6-{[5-(isopropylsulphonyl)-2-methoxyphenyl]amino}-1,3-dimethyl-3,4-dihydropyrido[2,3-b]pyrazin-2(1H)-one.

¹H-NMR: (400 MHz, 25° C., DMSO-d6): δ=1.09 (d, 3H); 1.12-1.18 (m, 6H); 1.20 (d, 3H); 1.29 (d, 3H); 3.18-3.28 (m, 4H); 3.98 (s, 3H); 4.24 (q, 1H); 4.81 (sept, 1H); 6.59 (d, 1H); 7.19 (d, 1H); 7.27 (d, 1H); 7.30 (dd, 1H); 8.16 (s, 1H); 8.95 (d, 1H).

### Example 17

### 1,3-Dimethyl-6-{[3-(methylsulphonyl)phenyl]amino}-4-phenyl-3,4-dihydropyrido[2,3-b]pyrazin-2(1H)-one

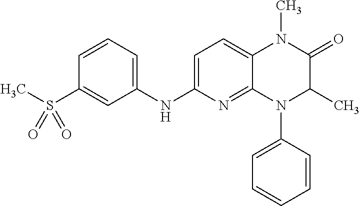

A mixture of 150 mg of intermediate 19, 127 mg of 3-(methylsulphonyl)aniline hydrochloride (CAS 80213-28-1), 22.7 mg of tris(dibenzylideneacetone)dipalladium(0) (CAS 51364-51-3), 242 mg of caesium carbonate and 28.7 mg of Xanthphos (CAS 161265-03-8) in 4.2 ml of dioxane was stirred under an argon atmosphere at 120° C. for 17 hours. A further 22.7 mg of tris(dibenzylideneacetone)dipalladium(0) and 28.7 mg of Xanthphos were added and the mixture was stirred for a further 8 hours under an argon atmosphere at 120° C. The mixture was added to water and extracted twice with ethyl acetate. The organic phase was washed with saturated aqueous sodium chloride solution, dried over sodium sulphate and the solvent was removed under reduced pressure. The residue was purified by RP-HPLC chromatography (column: X-Bridge C18 5 μm 100×30 mm, mobile phase: acetonitrile/water (0.1% by volume formic acid) gradient). This gave 111 mg of 1,3-dimethyl-6-{[3-(methylsulphonyl)phenyl]amino}-4-phenyl-3,4-dihydropyrido[2,3-b]pyrazin-2(1H)-one

¹H-NMR: (400 MHz, 25° C., DMSO-d6): δ=1.30 (d, 3H); 3.04 (s, 3H); 3.30 (s, 3H); 4.56 (q, 1H); 6.42 (d, 1H); 7.07 (t, 1H); 7.19-7.27 (m, 2H); 7.33-7.38 (m, 2H); 7.40-7.48 (m, 3H); 7.55 (t, 1H); 7.86 (ddd, 1H); 9.29 (s, 1H).

### Example 18

### 3′-{[(3R)-1,3-Dimethyl-2-oxo-4-(tetrahydro-2H-pyran-4-yl)-1,2,3,4-tetrahydropyrido[2,3-b]pyrazin-6-yl]amino}biphenyl-4-carbonitrile

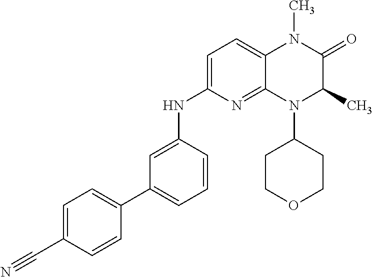 [SMILES: C[C@@H]1C(=O)N(C)c2ccc(Nc3cccc(-c4ccc(C#N)cc4)c3)nc2N1C1CCOCC1]

A mixture of 150 mg of intermediate 4, 187 mg of 3′-aminobiphenyl-4-carbonitrile (CAS 149505-72-6), 21 mg of palladium(II) acetate (CAS 3375-31-3), 785 mg of caesium carbonate and 60 mg of (+)-BINAP in 6.4 ml of toluene was stirred at 120° C. under an argon atmosphere for 10 hours. The mixture was added to water and extracted twice with ethyl acetate. The organic phase was washed with saturated aqueous sodium chloride solution, dried over sodium sulphate and the solvent was removed under reduced pressure. The residue was purified by RP-HPLC chromatography (column: X-Bridge C18 5 μm 100×30 mm, mobile phase: acetonitrile/water (0.1% by volume formic acid) gradient). This gave 115 mg of 3′-{[(3R)-1,3-dimethyl-2-oxo-4-(tetrahydro-2H-pyran-4-yl)-1,2,3,4-tetrahydropyrido[2,3-b]pyrazin-6-yl]amino}biphenyl-4-carbonitrile.

¹H-NMR: (400 MHz, 25° C., DMSO-d6): δ=1.07 (d, 3H); 1.50-1.56 (m, 1H); 1.66 (qd, 1H); 1.78-1.88 (m, 2H); 2.89 (dt, 1H); 3.12 (dt, 1H); 3.20 (s, 3H); 3.66-3.72 (m, 1H); 3.79-3.85 (m, 1H); 4.20 (q, 1H); 4.31 (tt, 1H); 6.27 (d, 1H); 7.13-7.17 (m, 1H); 7.27 (d, 1H); 7.36 (t, 1H); 7.64-7.68 (m, 1H); 7.81 (d, 2H); 7.88 (t, 1H); 7.92 (d, 2H); 8.96 (s, 1H).

### Example 19

### (3R)-6-{[3-(Isopropylsulphonyl)phenyl]amino}-1,3-dimethyl-4-(1-methylpiperidin-4-yl)-3,4-dihydropyrido[2,3-b]pyrazin-2(1H)-one

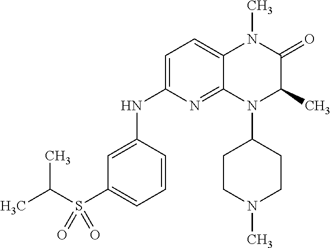 [SMILES: CC(C)S(=O)(=O)c1cccc(Nc2ccc3c(n2)N(C2CCN(C)CC2)[C@H](C)C(=O)N3C)c1]

A mixture of 150 mg of intermediate 15, 129 mg of 3-(propan-2-ylsulphonyl)aniline (CAS 170856-37-8), 21.1 mg of tris(dibenzylideneacetone)dipalladium(0) (CAS 51364-51-3), 226 mg of caesium carbonate and 26.7 mg of Xanthphos (CAS 161265-03-8) in 3.9 ml of dioxane was stirred under an argon atmosphere at 120° C. for 4 hours. A further 21.1 mg of tris(dibenzylideneacetone)dipalladium(0) and 26.7 mg of Xanthphos were added and the mixture was stirred for a further 4 hours under an argon atmosphere at 120° C. The mixture was added to water and extracted twice with ethyl acetate. The organic phase was washed with saturated aqueous sodium chloride solution, dried over sodium sulphate and the solvent was removed under reduced pressure. The residue was purified by RP-HPLC chromatography (column: X-Bridge C18 5 μm 100×30 mm, mobile phase: acetonitrile/water (0.1% by volume formic acid) gradient). This gave 68 mg of (3R)-6-{[3-(isopropylsulphonyl)phenyl]amino}-1,3-dimethyl-4-(1-methylpiperidin-4-yl)-3,4-dihydropyrido[2,3-b]pyrazin-2(1H)-one.

¹H-NMR: (400 MHz, 25° C., DMSO-d6): δ=1.07 (d, 3H); 1.17 (d, 6H); 1.59 (bd, 1H); 1.76 (qd, 1H); 1.89 (qd, 1H); 1.98 (bd, 1H); 2.14-2.29 (m+s, 5H); 2.85-2.96 (m, 2H); 3.21 (s, 3H); 3.31-3.42 (m, 1H); 4.17-4.29 (m, 2H); 6.28 (d, 1H); 7.26 (d, 1H); 7.29 (d, 1H); 7.48 (t, 1H); 7.88 (t, 1H); 8.16-8.20 (m, 1H); 9.28 (s, 1H).

Optical rotation [α]_{D}²⁰=−214.3°+/−2.22° (c=7.0 mg/ml, methanol).

### Example 20

### (3R)-6-{[5-(Isopropylsulphonyl)-2-(trifluoromethoxy)phenyl]amino}-1,3-dimethyl-4-(1-methylpiperidin-4-yl)-3,4-dihydropyrido[2,3-b]pyrazin-2(1H)-one

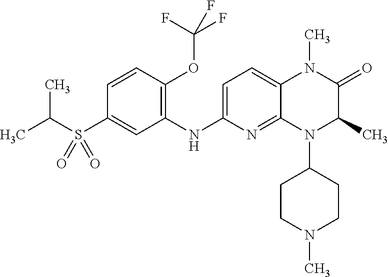 [SMILES: CC(C)S(=O)(=O)c1ccc(OC(F)(F)F)c(Nc2ccc3c(n2)N(C2CCN(C)CC2)[C@H](C)C(=O)N3C)c1]

A mixture of 150 mg of intermediate 15, 170 mg of intermediate 34, 21.1 mg of tris(dibenzylideneacetone)dipalladium(0) (CAS 51364-51-3), 226 mg of caesium carbonate, 18.1 mg of (2′-aminobiphenyl-2-yl)(chloro)palladium-dicyclohexyl[2′,4′,6′-tri(propan-2-yl)biphenyl-2-yl]phosphane (1:1) (CAS 1310584-14-5, commercially available) and 26.7 mg of Xanthphos (CAS 161265-03-8) in 3.9 ml of dioxane were stirred for 4 hours at 120° C. under an argon atmosphere. The mixture was added to water and extracted twice with ethyl acetate. The organic phase was washed with saturated aqueous sodium chloride solution, dried over sodium sulphate and the solvent was removed under reduced pressure. The residue was purified by chromatography on silica gel (dichloromethane/methanol gradient up to 20% methanol content). This gave 190 mg of (3R)-6-{[5-(isopropylsulphonyl)-2-(trifluoromethoxy)phenyl]amino}-1,3-dimethyl-4-(1-methylpiperidin-4-yl)-3,4-dihydropyrido[2,3-b]pyrazin-2(1H)-one.

¹H-NMR: (400 MHz, 25° C., DMSO-d6): δ=1.06 (d, 3H); 1.12-1.17 (m, 6H); 1.56 (bd, 1H); 1.67 (qd, 1H); 1.76-1.95 (m, 2H); 2.20-2.31 (m, 4H); 2.79-2.93 (m, 2H); 3.22 (s, 3H); 3.46 (sp, 1H); 4.15-4.28 (m, 2H); 6.58 (d, 1H); 7.34 (d, 1H); 7.41 (dd, 1H); 7.57-7.62 (m, 1H); 8.56 (d, 1H); 8.77 (s, 1H).

Optical rotation [α]_{D}²⁰=−242.9°+/−1.34° (c=7.0 mg/ml, methanol).

### Example 21

(3R)-6-({2-Methoxy-5-[(trifluoromethyl)sulphonyl]phenyl}amino)-1,3-dimethyl-4-(1-methylpiperidin-4-yl)-3,4-dihydropyrido[2,3-b]pyrazin-2(1H)-one

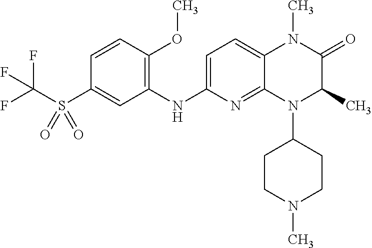 [SMILES: COc1ccc(S(=O)(=O)C(F)(F)F)cc1Nc1ccc2c(n1)N(C1CCN(C)CC1)[C@H](C)C(=O)N2C]

A mixture of 150 mg of intermediate 15, 186 mg of 2-methoxy-5-[(trifluoromethyl)sulphonyl]aniline (CAS 780-90-5), 238 mg of caesium carbonate and 38 mg of (2′-aminobiphenyl-2-yl)(chloro)palladium-dicyclohexyl[2′,4′,6′-tri(propan-2-yl)biphenyl-2-yl]phosphane (1:1) (CAS 1310584-14-5, commercially available) in 7.5 ml of dioxane were stirred for 3 hours at 120° C. under an argon atmosphere. A further 38 mg of (2′-aminobiphenyl-2-yl)(chloro)palladium-dicyclohexyl[2′,4′,6′-tri(propan-2-yl)biphenyl-2-yl]phosphane (1:1) were added and the mixture was stirred at 120° C. for a further 5 hours. The mixture was added to water and extracted twice with ethyl acetate. The organic phase was washed with saturated aqueous sodium chloride solution, dried over sodium sulphate and the solvent was removed under reduced pressure. The residue was purified by RP-HPLC chromatography (column: X-Bridge C18 5 μm 100×30 mm, mobile phase: acetonitrile/water (0.1% by volume formic acid) gradient). This gave 65 mg of (3R)-6-({2-methoxy-5-[(trifluoromethyl)sulphonyl]phenyl}amino)-1,3-dimethyl-4-(1-methylpiperidin-4-yl)-3,4-dihydropyrido[2,3-b]pyrazin-2(1H)-one.

¹H-NMR: (400 MHz, 25° C., DMSO-d6): δ=1.05 (d, 3H); 1.50 (bd, 1H); 1.62-1.80 (m, 2H); 1.85 (bd, 1H); 2.05-2.18 (m+s, 5H); 2.70-2.79 (m, 2H); 3.21 (s, 3H); 4.04 (s, 3H); 4.18 (q, 1H); 4.26 (tt, 1H); 6.64 (d, 1H); 7.33 (d, 1H); 7.37 (d, 1H); 7.56-7.63 (m, 1H); 8.38 (s, 1H); 8.60 (d, 1H).

Optical rotation [α]_{D}²⁰=−155.3°+/−0.57° (c=8.0 mg/ml, methanol).

### Example 22

### (3R)-6-{[2-Methoxy-5-(tetrahydro-2H-pyran-4-ylsulphonyl)phenyl]amino}-1,3-dimethyl-4-(1-methylpiperidin-4-yl)-3,4-dihydropyrido[2,3-b]pyrazin-2(1H)-one

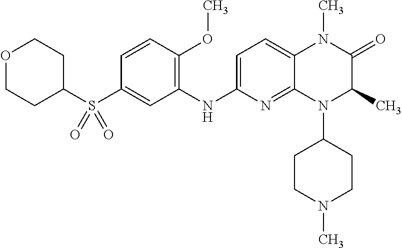 [SMILES: COc1ccc(S(=O)(=O)C2CCOCC2)cc1Nc1ccc2c(n1)N(C1CCN(C)CC1)[C@H](C)C(=O)N2C]

A mixture of 150 mg of intermediate 15, 198 mg of intermediate 38, 237 mg of caesium carbonate and 38 mg of (2′-aminobiphenyl-2-yl)(chloro)palladium-dicyclohexyl[2′,4′,6′-tri(propan-2-yl)biphenyl-2-yl]phosphane (1:1) (CAS 1310584-14-5, commercially available) in 7.5 ml of dioxane were stirred for 9 hours at 120° C. under an argon atmosphere. The mixture was added to water and extracted twice with ethyl acetate. The organic phase was washed with saturated aqueous sodium chloride solution, dried over sodium sulphate and the solvent was removed under reduced pressure. The residue was purified by RP-HPLC chromatography (column: X-Bridge C18 5 μm 100×30 mm, mobile phase: acetonitrile/water (0.1% by volume formic acid) gradient). This gave 95 mg of (3R)-6-{[2-methoxy-5-(tetrahydro-2H-pyran-4-ylsulphonyl)phenyl]amino}-1,3-dimethyl-4-(1-methylpiperidin-4-yl)-3,4-dihydropyrido[2,3-b]pyrazin-2(1H)-one.

¹H-NMR: (400 MHz, 25° C., DMSO-d6): δ=1.05 (d, 3H); 1.47-1.60 (m, 3H); 1.61-1.83 (m, 4H); 1.89 (bd, 1H); 2.20 (s, 3H); 2.22-2.31 (m, 2H); 2.71-2.82 (m, 2H); 3.19-3.29 (m+s, 5H); 3.33-3.45 (m, 2H); 3.85-3.90 (m, 2H); 3.96 (s, 3H); 4.16 (q, 1H); 4.31 (tt, 1H); 6.56 (d, 1H); 7.21 (d, 1H); 7.29 (d, 1H); 7.33 (dd, 1H); 8.14 (s, 1H); 8.51 (d, 1H).

Optical rotation [α]_{D}²⁰=−222°+/−0.2° (c=7.0 mg/ml, methanol).

### Example 23

### (3R)-6-{[5-(Allylsulphonyl)-2-methoxyphenyl]amino}-1,3-dimethyl-4-(1-methylpiperidin-4-yl)-3,4-dihydropyrido[2,3-b]pyrazin-2(1H)-one

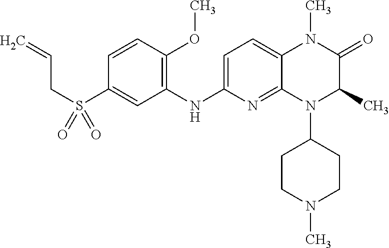 [SMILES: C=CCS(=O)(=O)c1ccc(OC)c(Nc2ccc3c(n2)N(C2CCN(C)CC2)[C@H](C)C(=O)N3C)c1]

In the preparation of example 12, 50 mg of (3R)-6-{[5-(allylsulphonyl)-2-methoxyphenyl]amino}-1,3-dimethyl-4-(1-methylpiperidin-4-yl)-3,4-dihydropyrido[2,3-b]pyrazin-2(1H)-one were obtained as a by-product.

¹H-NMR: (400 MHz, 25° C., DMSO-d6): δ=1.05 (d, 3H); 1.50 (bd, 1H); 1.63 (qd, 1H); 1.76 (qd, 1H); 1.87 (bd, 1H); 2.10-2.22 (m+s, 5H); 2.65-2.75 (m, 2H); 3.21 (s, 3H); 3.94-4.02 (m+s, 1H); 4.17 (q, 1H); 4.26 (tt, 1H); 5.20 (dd, 1H); 5.28 (dd, 1H); 5.61-5.73 (m, 1H); 6.54 (d, 1H); 7.178 (d, 1H); 7.28 (d, 1H); 7.34 (dd, 1H); 8.10 (s, 1H); 8.48 (d, 1H).

### Example 24

### tert-Butyl 4-[(3R)-6-{[5-(isopropylsulphonyl)-2-methoxyphenyl]amino}-1,3-dimethyl-2-oxo-2,3-dihydropyrido[2,3-b]pyrazin-4(1H)-yl]piperidine-1-carboxylate

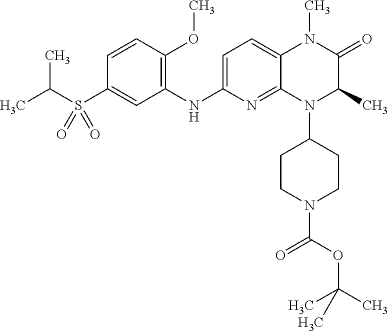 [SMILES: COc1ccc(S(=O)(=O)C(C)C)cc1Nc1ccc2c(n1)N(C1CCN(C(=O)OC(C)(C)C)CC1)[C@H](C)C(=O)N2C]

A mixture of 150 mg of tert-butyl 4-[(3R)-6-chloro-1,3-dimethyl-2-oxo-2,3-dihydropyrido[2,3-b]pyrazin-4(1H)-yl]piperidine-1-carboxylate (CAS 1615234-64-4, preparation described in WO2014095774, Intermediate 56), 131 mg of intermediate 30, 17 mg of tris(dibenzylideneacetone)dipalladium(0) (CAS 51364-51-3), 173 mg of caesium carbonate and 22 mg of Xanthphos (CAS 161265-03-8) in 10 ml of dioxane was stirred under an argon atmosphere at 120° C. for 2 hours. 15 mg of (2′-aminobiphenyl-2-yl)(chloro)palladium-dicyclohexyl[2′,4′,6′-tri(propan-2-yl)biphenyl-2-yl]phosphane (1:1) (CAS 1310584-14-5, commercially available) were added and the mixture was stirred at 120° C. for a further 4 hours. The mixture was diluted with dichloromethane and applied to Isolute® by evaporating the solvent. This residue was purified by chromatography on silica gel (dichloromethane/methanol gradient up to 3% methanol content). This gave 130 mg of tert-butyl 4-[(3R)-6-{[5-isopropylsulphonyl)-2-methoxyphenyl]amino}-1,3-dimethyl-2-oxo-2,3-dihydropyrido[2,3-b]pyrazin-4(1H)-yl]piperidine-1-carboxylate.

¹H-NMR: (400 MHz, 25° C., DMSO-d6, some signals are under the water signal): δ=1.04 (d, 3H); 1.10-1.15 (m, 6H); 1.40 (s, 9H); 1.44-1.66 (m, 3H); 1.89 (bd, 1H); 3.21 (s, 3H); 3.89-4.00 (m+s, 4H); 4.18 (q, 1H); 4.50 (tt, 1H); 6.58 (d, 1H); 7.21 (d, 1H); 7.30 (d, 1H); 7.33 (dd, 1H); 8.13 (s, 1H); 8.58 (d, 1H).

### Example 25

### tert-Butyl 4-[(3R)-6-{[2-methoxy-5-(methylsulphonyl)phenyl]amino}-1,3-dimethyl-2-oxo-2,3-dihydropyrido[2,3-b]pyrazin-4(1H)-yl]piperidine-1-carboxylate

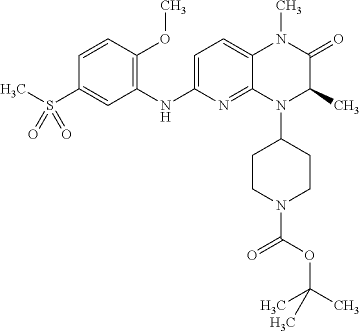 [SMILES: COc1ccc(S(C)(=O)=O)cc1Nc1ccc2c(n1)N(C1CCN(C(=O)OC(C)(C)C)CC1)[C@H](C)C(=O)N2C]

A mixture of 150 mg of tert-butyl 4-[(3R)-6-chloro-1,3-dimethyl-2-oxo-2,3-dihydropyrido[2,3-b]pyrazin-4(1H)-yl]piperidine-1-carboxylate (CAS 1615234-64-4, preparation described in WO2014095774, Intermediate 56), 114 mg of 2-methoxy-5-(methylsulphonyl)aniline (CAS 20945-70-4), 17 mg of tris(dibenzylideneacetone)dipalladium(0) (CAS 51364-51-3), 173 mg of caesium carbonate and 22 mg of Xanthphos (CAS 161265-03-8) in 10 ml of dioxane was stirred under an argon atmosphere at 120° C. for 2 hours. 15 mg of (2′-aminobiphenyl-2-yl)(chloro)palladium-dicyclohexyl[2′,4′,6′-tri(propan-2-yl)biphenyl-2-yl]phosphane (1:1) (CAS 1310584-14-5, commercially available) were added and the mixture was stirred at 120° C. for a further 4 hours. The mixture was diluted with dichloromethane and applied to Isolute® by evaporating the solvent. This residue was purified by chromatography on silica gel (dichloromethane/methanol gradient up to 3% methanol content). This gave 130 mg of tert-butyl 4-[(3R)-6-{[2-methoxy-5-(methylsulphonyl)phenyl]amino}-1,3-dimethyl-2-oxo-2,3-dihydropyrido[2,3-b]pyrazin-4(1H)-yl]piperidine-1-carboxylate.

¹H-NMR: (400 MHz, 25° C., DMSO-d6): δ=1.04 (d, 3H); 1.40 (s, 9H); 1.43-1.64 (m, 3H); 1.91 (bd, 1H); 2.82-3.06 (m, 2H); 3.12 (s, 3H); 3.21 (s, 3H); 3.88-4.03 (m+s, 5H); 4.18 (q, 1H); 4.51 (tt, 1H); 6.59 (d, 1H); 7.20 (d, 1H); 7.30 (d, 1H); 7.41 (dd, 1H); 8.16 (s, 1H); 8.68 (d, 1H).

### Example 26

### (3R)-6-{[5-(Isopropylsulphonyl)-2-methoxyphenyl]amino}-1,3-dimethyl-4-(piperidin-4-yl)-3,4-dihydropyrido[2,3-b]pyrazin-2(1H)-one

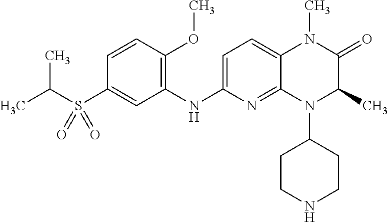 [SMILES: COc1ccc(S(=O)(=O)C(C)C)cc1Nc1ccc2c(n1)N(C1CCNCC1)[C@H](C)C(=O)N2C]

A solution of 130 mg of Example 24 in 10 ml of dichloromethane and 0.22 ml of trifluoroacetic acid was stirred at RT for 14 hours. With addition of toluene, the solvent was removed under reduced pressure and the residue was purified by RP-HPLC (column: X-Bridge C18 5 μm 100×30 mm, mobile phase: acetonitrile/water (0.2% by volume ammonia (35%) gradient). This gave 80 mg of (3R)-6-{[5-(isopropylsulphonyl)-2-methoxyphenyl]amino}-1,3-dimethyl-4-(piperidin-4-yl)-3,4-dihydropyrido[2,3-b]pyrazin-2(1H)-one.

¹H-NMR (400 MHz, DMSO-d6, some signals are obscured by the water signal): δ=1.05 (d, 3H); 1.13 (d, 6H); 1.41-1.63 (m, 3H); 1.83 (bd, 1H); 2.62-2.72 (m, 2H); 2.85-2.95 (m, 2H); 3.21 (s, 3H); 3.96 (s, 3H); 4.18 (q, 1H); 4.41 (tt, 1H); 6.54 (d, 1H); 7.20 (d, 1H); 7.28 (d, 1H); 7.33 (dd, 1H); 8.09 (s, 1H); 8.55 (d, 1H).

### Example 27

### (3R)-6-{[2-Methoxy-5-(methylsulphonyl)phenyl]amino}-1,3-dimethyl-4-(piperidin-4-yl)-3,4-dihydropyrido[2,3-b]pyrazin-2(1H)-one

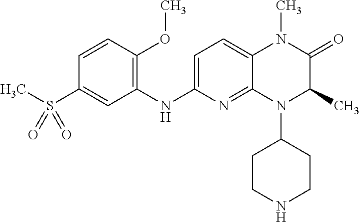

A solution of 170 mg of Example 25 in 10 ml of dichloromethane and 0.3 ml of trifluoroacetic acid was stirred at RT for 14 hours. With addition of toluene, the solvent was removed under reduced pressure and the residue was purified by RP-HPLC (column: X-Bridge C18 5 μm 100×30 mm, mobile phase: acetonitrile/water (0.2% by volume ammonia (35%) gradient). This gave 90 mg of (3R)-6-{[2-methoxy-5-(methylsulphonyl)phenyl]amino}-1,3-dimethyl-4-(piperidin-4-yl)-3,4-dihydropyrido[2,3-b]pyrazin-2(1H)-one.

¹H-NMR (400 MHz, DMSO-d6): δ=1.06 (d, 3H); 1.42-1.65 (m, 3H); 1.86 (bd, 1H); 2.58-2.71 (m, 2H); 2.85-2.94 (m, 2H); 3.11 (s, 3H); 3.21 (s, 3H); 3.96 (s, 3H); 4.18 (q, 1H); 4.42 (tt, 1H); 6.56 (d, 1H); 7.19 (d, 1H); 7.28 (d, 1H); 7.41 (dd, 1H); 8.12 (s, 1H); 8.63 (d, 1H).

### Example 28

### (3R)-6-{[5-(Isopropylsulphonyl)-2-methoxyphenyl]amino}-1,3-dimethyl-4-[1-(2,2,2-trifluoroethyl)piperidin-4-yl]-3,4-dihydropyrido[2,3-b]pyrazin-2(1H)-one

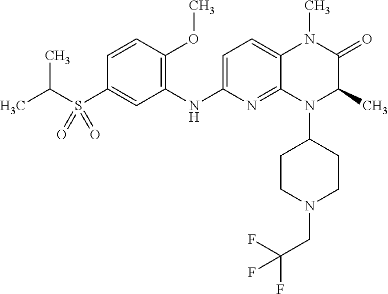 [SMILES: COc1ccc(S(=O)(=O)C(C)C)cc1Nc1ccc2c(n1)N(C1CCN(CC(F)(F)F)CC1)[C@H](C)C(=O)N2C]

A mixture of 150 mg of intermediate 39, 119 mg of intermediate 30, 259 mg of caesium carbonate and 63 mg of (2′-aminobiphenyl-2-yl)(chloro)palladium-dicyclohexyl[2′,4′,6′-tri(propan-2-yl)biphenyl-2-yl]phosphane (1:1) (CAS 1310584-14-5, commercially available) in 3.2 ml of dioxane was heated to 110° C. in a microwave oven for 1 hour under an argon atmosphere. The mixture was filtered and the solvent was removed under reduced pressure. The residue was purified by RP-HPLC chromatography (column: X-Bridge C18 5 μm 100×30 mm, mobile phase: acetonitrile/water (0.1% by volume formic acid) gradient). This gave 57 mg of (3R)-6-{[5-(isopropylsulphonyl)-2-methoxyphenyl]amino}-1,3-dimethyl-4-[1-(2,2,2-trifluoroethyl)piperidin-4-yl]-3,4-dihydropyrido[2,3-b]pyrazin-2(1H)-one.

¹H-NMR: (400 MHz, 25° C., DMSO-d6): δ=1.05 (d, 3H); 1.11-1.17 (m, 6H); 1.50 (bd, 1H); 1.66 (qd, 1H); 1.71-1.89 (m, 2H); 2.58-2.70 (m, 2H); 2.81-2.92 (m, 2H); 3.14 (dq, 2H); 3.21 (s, 3H); 3.28 (sp, 1H); 3.96 (s, 3H); 4.20 (q, 1H); 4.31 (tt, 1H); 6.55 (d, 1H); 7.20 (d, 1H); 7.29 (d, 1H); 7.34 (dd, 1H); 8.11 (s, 1H); 8.50 (d, 1H).

### Example 29

### (3R)-4-[1-(2,2-Difluoroethyl)piperidin-4-yl]-6-{[5-(isopropylsulphonyl)-2-methoxyphenyl]amino}-1,3-dimethyl-3,4-dihydropyrido[2,3-b]pyrazin-2(1H)-one

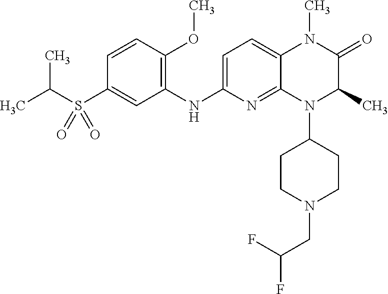 [SMILES: COc1ccc(S(=O)(=O)C(C)C)cc1Nc1ccc2c(n1)N(C1CCN(CC(F)F)CC1)[C@H](C)C(=O)N2C]

A mixture of 134 mg of intermediate 40, 111 mg of intermediate 30, 244 mg of caesium carbonate and 59 mg of (2′-aminobiphenyl-2-yl)(chloro)palladium-dicyclohexyl[2′,4′,6′-tri(propan-2-yl)biphenyl-2-yl]phosphane (1:1) (CAS 1310584-14-5, commercially available) in 3 ml of dioxane was heated to 110° C. in a microwave oven for 1 hour under an argon atmosphere. The mixture was filtered and the solvent was removed under reduced pressure. The residue was pre-purified by RP-HPLC chromatography (column: X-Bridge C18 5 μm 100×30 mm, mobile phase: acetonitrile/water (0.1% by volume formic acid) gradient). The resulting crude product was purified further by chromatography on silica gel (dichloromethane/methanol gradient up to 1% methanol content). This gave 57 mg of (3R)-4-[1-(2,2-difluoroethyl)piperidin-4-yl]-6-{[5-(isopropylsulphonyl)-2-methoxyphenyl]amino}-1,3-dimethyl-3,4-dihydropyrido[2,3-b]pyrazin-2(1H)-one.

¹H-NMR: (400 MHz, 25° C., DMSO-d6, some signals masked by DMSO signal): δ=1.05 (d, 3H); 1.10-1.18 (m, 6H); 1.51 (bd, 1H); 1.63 (qd, 1H); 1.75 (qd, 1H); 1.86 (bd, 1H); 2.71 (dt, 2H); 2.80-2.90 (m, 2H); 3.21 (s, 3H); 3.28 (sp, 1H); 3.96 (s, 3H); 4.18 (q, 1H); 4.30 (tt, 1H); 6.10 (tt, 1H); 6.55 (d, 1H); 7.20 (d, 1H); 7.29 (d, 1H); 7.34 (dd, 1H); 8.11 (s, 1H); 8.49 (d, 1H).

Optical rotation [α]_{D}²⁰=−201.9°+/−0.31° (c=6.0 mg/ml, methanol).

### Example 30

### (3R)-6-{[3-(Isopropylsulphonyl)phenyl]amino}-1,3-dimethyl-4-[1-(3,3,3-trifluoropropyl)piperidin-4-yl]-3,4-dihydropyrido[2,3-b]pyrazin-2(1H)-one

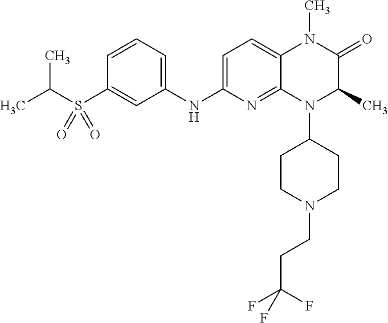 [SMILES: CC(C)S(=O)(=O)c1cccc(Nc2ccc3c(n2)N(C2CCN(CCC(F)(F)F)CC2)[C@H](C)C(=O)N3C)c1]

A mixture of 180 mg of intermediate 41, 137 mg of 3-(propan-2-ylsulphonyl)aniline (CAS 170856-37-8), 21 mg of tris(dibenzylideneacetone)dipalladium(0) (CAS 51364-51-3), 210 mg of caesium carbonate and 27 mg of Xanthphos (CAS 161265-03-8) in 10 ml of dioxane was stirred under an argon atmosphere at 120° C. for 2 hours. 18 mg of (2′-aminobiphenyl-2-yl)(chloro)palladium-dicyclohexyl[2′,4′,6′-tri(propan-2-yl)biphenyl-2-yl]phosphane (1:1) (CAS 1310584-14-5, commercially available) were added and the mixture was stirred at 120° C. for a further 4 hours. The mixture was diluted with dichloromethane and applied to Isolute® by evaporating the solvent. This residue was purified by chromatography on silica gel (dichloromethane/methanol gradient up to 10% methanol content). This gave 130 mg of (3R)-6-{[3-(isopropylsulphonyl)phenyl]amino}-1,3-dimethyl-4-[1-(3,3,3-trifluoropropyl)piperidin-4-yl]-3,4-dihydropyrido[2,3-b]pyrazin-2(1H)-one.

¹H-NMR: (400 MHz, 25° C., DMSO-d6, some signals masked by water and DMSO): δ=1.07 (d, 3H); 1.11-1.20 (m, 6H); 1.60 (bd, 1H); 1.68 (qd, 1H); 1.82 (qd, 1H); 1.99 (bd, 1H); 2.14-2.25 (m, 2H); 2.90-3.02 (m, 2H); 3.20 (s, 3H); 4.16-4.28 (m, 2H); 6.27 (d, 1H); 7.26 (dd, 1H); 7.29 (d, 1H); 7.50 (t, 1H); 7.69 (t, 1H); 8.14 (dd, 1H); 9.27 (s, 1H).

chiral HPLC: Rt=7.12 min, (92% ee)

Instrument: Agilent HPLC 1260; column: Chiralpak ID 3 μm 100×4.6 mm; mobile phase: hexane (+0.1 vol % diethylamine)/2-propanol gradient 5-50% 2-propanol content; flow rate 1 ml/min; temperature: 25° C.; DAD 996 scan: 280 nm.

### Example 31

### (3R)-6-{[5-(Isopropylsulphonyl)-2-methoxyphenyl]amino}-1,3-dimethyl-4-[1-(3,3,3-trifluoropropyl)piperidin-4-yl]-3,4-dihydropyrido[2,3-b]pyrazin-2(1H)-one

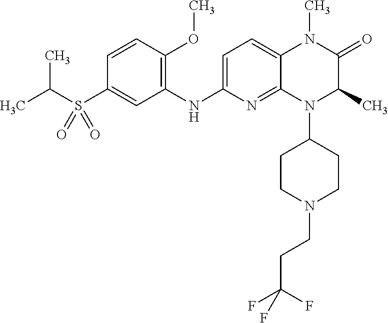 [SMILES: COc1ccc(S(=O)(=O)C(C)C)cc1Nc1ccc2c(n1)N(C1CCN(CCC(F)(F)F)CC1)[C@H](C)C(=O)N2C]

A mixture of 150 mg of intermediate 41, 132 mg of intermediate 30, 18 mg of tris(dibenzylideneacetone)dipalladium(0) (CAS 51364-51-3), 175 mg of caesium carbonate and 22 mg of Xanthphos (CAS 161265-03-8) in 8 ml of dioxane was stirred under an argon atmosphere at 120° C. for 2 hours. 15 mg of (2′-aminobiphenyl-2-yl)(chloro)palladium-dicyclohexyl[2′,4′,6′-tri(propan-2-yl)biphenyl-2-yl]phosphane (1:1) (CAS 1310584-14-5, commercially available) were added and the mixture was stirred at 120° C. for a further 4 hours. The mixture was diluted with dichloromethane and applied to Isolute® by evaporating the solvent. This residue was purified by chromatography on silica gel (dichloromethane/methanol gradient up to 10% methanol content). This gave 100 mg of (3R)-6-{[5-(isopropylsulphonyl)-2-methoxyphenyl]amino}-1,3-dimethyl-4-[1-(3,3,3-trifluoropropyl)piperidin-4-yl]-3,4-dihydropyrido[2,3-b]pyrazin-2(1H)-one.

¹H-NMR: (400 MHz, 25° C., DMSO-d6, some signals under DMSO): δ=1.04 (d, 3H); 1.09-1.18 (m, 6H); 1.49-1.66 (m, 2H); 1.72 (qd, 1H); 1.88 (bd, 1H); 2.22-2.35 (m, 2H); 2.75-2.87 (m, 2H); 3.20 (s, 3H); 3.28 (sp, 1H); 3.96 (s, 3H); 4.16 (q, 1H); 4.30 (tt, 1H); 6.55 (d, 1H); 7.20 (d, 1H); 7.29 (d, 1H); 7.34 (dd, 1H); 8.12 (s, 1H); 8.50 (d, 1H).

chiral HPLC: Rt=3.59 min (>95% ee)

Instrument: Agilent HPLC 1260; column: Chiralpak IB 3 μm 100×4.6 mm; Eluent: water (+0.4 vol % formic acid)/acetonitrile gradient 20-90% acetonitrile content; flow rate 1.4 ml/min; temperature: 25° C.; MWD: 254 nm.

### Example 32

### (3R)-6-{[5-(Ethylsulphonyl)-2-(4-fluorophenoxy)phenyl]amino}-1,3-dimethyl-4-(1-methylpiperidin-4-yl)-3,4-dihydropyrido[2,3-b]pyrazin-2(1H)-one

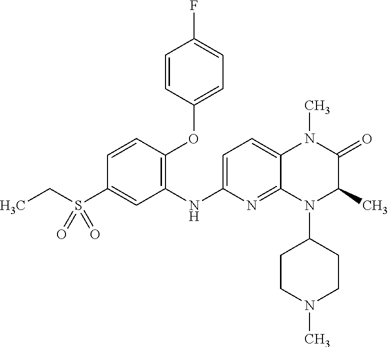 [SMILES: CCS(=O)(=O)c1ccc(Oc2ccc(F)cc2)c(Nc2ccc3c(n2)N(C2CCN(C)CC2)[C@H](C)C(=O)N3C)c1]

A mixture of 150 mg of intermediate 15, 191 mg of intermediate 43, 21 mg of tris(dibenzylideneacetone)dipalladium(0) (CAS 51364-51-3), 226 mg of caesium carbonate and 26.7 mg of Xanthphos (CAS 161265-03-8) in 7.4 ml of dioxane was stirred under an argon atmosphere at 120° C. for 5 hours. A further 21 mg of tris(dibenzylideneacetone)dipalladium(0) and 26.7 mg of Xanthphos were added and the mixture was stirred for a further 7 hours at 120° C. The mixture was added to water and extracted twice with ethyl acetate. The organic phase was washed with saturated aqueous sodium chloride solution, dried over sodium sulphate and the solvent was removed under reduced pressure. The residue was purified by chromatography on silica gel (dichloromethane/methanol gradient up to 1% methanol content). This gave 84 mg of (3R)-6-{[5-(ethylsulphonyl)-2-(4-fluorophenoxy)phenyl]amino}-1,3-dimethyl-4-(1-methylpiperidin-4-yl)-3,4-dihydropyrido[2,3-b]pyrazin-2(1H)-one.

¹H-NMR: (400 MHz, 25° C., DMSO-d6): δ=1.07 (d, 3H); 1.12 (t, 3H); 1.58 (bd, 1H); 1.69 (qd, 1H); 1.82 (qd, 1H); 1.94 (bd, 1H); 2.16-2.30 (m, 4H); 2.75-2.88 (m, 2H); 3.19-3.26 (m+s, 5H); 4.19 (q, 1H); 4.32 (tt, 1H); 6.59 (d, 1H); 6.88 (d, 1H); 7.19-7.25 (m, 2H); 7.27-7.34 (m, 4H); 8.56 (s, 1H); 8.66 (d, 1H).

chiral HPLC: Rt=3.89 min, (95% ee)

Instrument: Agilent HPLC 1260; column: Chiralpak IF 3 μm 100×4.6 mm; mobile phase: water (+0.4 vol % formic acid)/acetonitrile gradient 20-90% acetonitrile content; flow rate 1.4 ml/min; temperature: 25° C.; MWD: 254 nm.

### Example 33

### (3R)-6-{[5-(Ethylsulphonyl)-2-phenoxyphenyl]amino}-1,3-dimethyl-4-(1-methylpiperidin-4-yl)-3,4-dihydropyrido[2,3-b]pyrazin-2(1H)-one

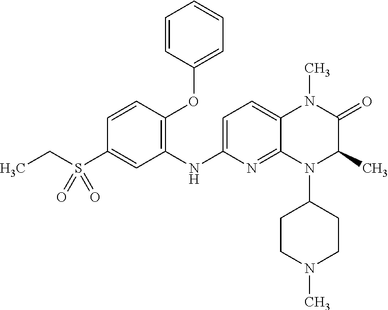 [SMILES: CCS(=O)(=O)c1ccc(Oc2ccccc2)c(Nc2ccc3c(n2)N(C2CCN(C)CC2)[C@H](C)C(=O)N3C)c1]

A mixture of 150 mg of intermediate 15, 179 mg of intermediate 45, 21 mg of tris(dibenzylideneacetone)dipalladium(0) (CAS 51364-51-3), 226 mg of caesium carbonate and 26.7 mg of Xanthphos (CAS 161265-03-8) in 7.4 ml of dioxane was stirred under an argon atmosphere at 120° C. for 7 hours. The mixture was added to water and extracted twice with ethyl acetate. The organic phase was washed with saturated aqueous sodium chloride solution, dried over sodium sulphate and the solvent was removed under reduced pressure. The residue was pre-purified by RP-HPLC chromatography (column: X-Bridge C18 5 μm 100×30 mm, mobile phase: acetonitrile/water (0.2% by volume ammonia (35%) gradient). This gave 62 mg of (3R)-6-{[5-(ethylsulphonyl)-2-phenoxyphenyl]amino}-1,3-dimethyl-4-(1-methylpiperidin-4-yl)-3,4-dihydropyrido[2,3-b]pyrazin-2(1H)-one.

¹H-NMR: (400 MHz, 25° C., DMSO-d6): δ=1.07 (d, 3H); 1.13 (t, 3H); 1.51 (bd, 1H); 1.65 (qd, 1H); 0.77 (qd, 1H); 1.90 (bd, 1H); 2.06-2.22 (m, 5H); 2.65-2.75 (m, 2H); 3.18-3.25 (m, 5H); 4.19 (q, 1H); 4.28 (tt, 1H); 6.57 (d, 1H); 6.90 (d, 1H); 7.13-7.18 (m, 2H); 7.25 (tt, 1H); 7.28-7.33 (m, 2H); 7.43-7.50 (m, 2H); 8.54 (s, 1H); 8.64 (d, 1H).

### Example 34

### (3R)-6-{[2-Methoxy-5-(tetrahydro-2H-pyran-4-ylsulphonyl)phenyl]amino}-1,3-dimethyl-4-(tetrahydro-2H-pyran-4-yl)-3,4-dihydropyrido[2,3-b]pyrazin-2(1H)-one

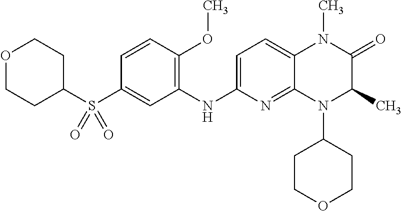 [SMILES: COc1ccc(S(=O)(=O)C2CCOCC2)cc1Nc1ccc2c(n1)N(C1CCOCC1)[C@H](C)C(=O)N2C]

A mixture of 150 mg of intermediate 4, 206 mg of intermediate 38, 248 mg of caesium carbonate and 40 mg of (2′-aminobiphenyl-2-yl)(chloro)palladium-dicyclohexyl[2′,4′,6′-tri(propan-2-yl)biphenyl-2-yl]phosphane (1:1) (CAS 1310584-14-5, commercially available) in 7.8 ml of dioxane was stirred for 5 hours at 120° C. under an argon atmosphere. The mixture was added to water and extracted twice with ethyl acetate. The organic phase was washed with saturated aqueous sodium chloride solution, dried over sodium sulphate and the solvent was removed under reduced pressure. The residue was purified by RP-HPLC chromatography (column: X-Bridge C18 5 μm 100×30 mm, mobile phase: acetonitrile/water (0.1% by volume formic acid) gradient). This gave 112 mg of (3R)-6-{[2-methoxy-5-(tetrahydro-2H-pyran-4-ylsulphonyl)phenyl]amino}-1,3-dimethyl-4-(tetrahydro-2H-pyran-4-yl)-3,4-dihydropyrido[2,3-b]pyrazin-2(1H)-one.

¹H-NMR: (400 MHz, 25° C., DMSO-d6): δ=1.06 (d, 3H); 1.44-1.58 (m, 3H); 1.62-1.89 (m, 5H); 3.21 (s, 3H); 3.24 (dt, 2H); 3.57 (dq, 2H); 3.77-3.92 (m, 4H); 3.96 (s, 3H); 4.21 (q, 1H); 4.55 (tt, 1H); 6.57 (d, 1H); 7.21 (d, 1H); 7.390 (d, 1H); 7.33 (dd, 1H); 8.14 (s, 1H); 8.57 (d, 1H).

Optical rotation [α]_{D}²⁰=−173.5°+/−0.73° (c=6.0 mg/ml, methanol).

The following examples in table 1 were prepared analogously to example 34.

TABLE 1

| Ex- | | | | |
|---|---|---|---|---|
| ample | | | | |
| No. | Structure | Name | Reactants | Analysis |
| 35 | 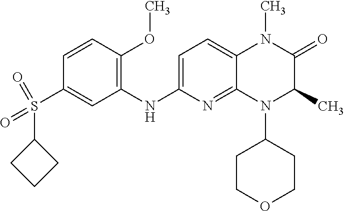 [SMILES: COc1ccc(S(=O)(=O)C2CCC2)cc1Nc1ccc2c(n1)N(C1CCOCC1)[C@H](C)C(=O)N2C] | (3R)-6-{[5- (Cyclobutylsulphonyl)-2- methoxyphenyl]amino}-1,3- dimethyl-4-(tetrahydro-2H- pyran-4-yl)-3,4- dihydropyrido[2,3- b]pyrazin-2(1H)-one | Intermediate 4, Intermediate 49 | ¹H-NMR: (400 MHz, 25° C., DMSO-d6): δ = 1.06 (d, 3H); 1.51 (bd, 1H); 1.63-1.94 (m, 5H); 2.01-2.14 (m, 2H); 2.23-2.35 (m, 2H); 3.21 (s, 3H); 3.53-3.62 (m, 2H); 3.80-3.87 (m, 2H); 3.92-4.03 (m + s, 4H); 4.21 (q, 1H); 4.57 (tt, 1H); 6.58 (d, 1H); 7.18 (d, 1H); 7.28- 7.34 (m, 2H); 8.16 (s, 1H); 8.62 (d, 1H). |
| 36 | 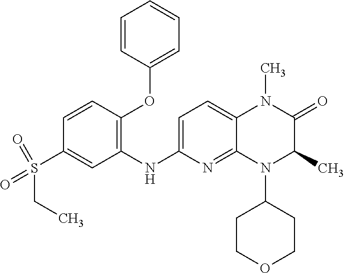 [SMILES: CCS(=O)(=O)c1ccc(Oc2ccccc2)c(Nc2ccc3c(n2)N(C2CCOCC2)[C@H](C)C(=O)N3C)c1] | (3R)-6-{[5- (Ethylsulphonyl)-2- phenoxyphenyl]amino}-1,3- dimethyl-4-(tetrahydro-2H- pyran-4-yl)-3,4- dihydropyrido[2,3- b]pyrazin-2(1H)-one | Intermediate 4, Intermediate 45 | ¹H-NMR: (400 MHz, 25° C., DMSO-d6): δ = 1.07 (d, 3H); 1.11 (t, 3H); 1.53 (bd, 1H); 1.70 (qd, 1H); 1.80 (qd, 1H); 1.89 (bd, 1H); 3.18-3.28 (m, 5H); 3.53 (dt, 1H); 3.59 (dt, 1H); 3.78-3.88 (m, 2H); 4.23 (q, 1H); 4.57 (tt, 1H); 6.61 (d, 1H); 6.89 (d, 1H); 7.14-7.19 (m, 2H); 7.25 (tt, 1H); 7.29- 7.34 (m, 2H); 7.44- 7.51 (m, 2H); 8.59 (s, 1H); 8.74 (d, 1H). |
| 37 | 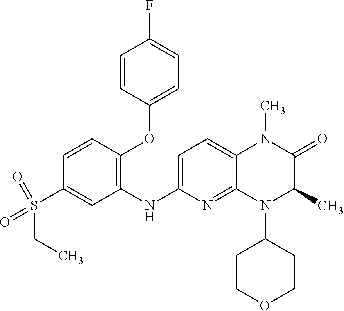 [SMILES: CCS(=O)(=O)c1ccc(Oc2ccc(F)cc2)c(Nc2ccc3c(n2)N(C2CCOCC2)[C@H](C)C(=O)N3C)c1] | (3R)-6-{[5- (Ethylsulphonyl)-2-(4- fluorophenoxy)phenyl] amino}-1,3-dimethyl-4- (tetrahydro-2H-pyran-4-yl)- 3,4-dihydropyrido[2,3- b]pyrazin-2(1H)-one | Intermediate 4, Intermediate 43 | ¹H-NMR: (400 MHz, 25° C., DMSO-d6): δ = 1.06-1.15 (m, 6H); 1.53 (bd, 1H); 1.70 (qd, 1H); 1.80 (qd, 1H); 1.88 (bd, 1H); 3.17-3.26 (m, 5H); 3.52 (dt, 1H); 3.59 (dt, 1H); 3.78-3.87 (m, 2H); 4.23 (q, 1H); 4.57 (tt, 1H); 6.61 (d, 1H); 6.87 (d, 1H); 7.19-7.26 (m, 2H); 7.27-7.35 (m, 4H); 8.60 (s, 1H); 8.73 (d, 1H). |
| 38 | 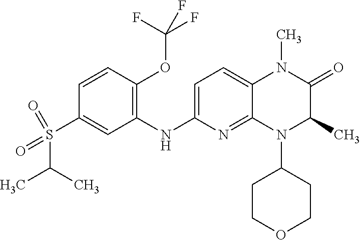 [SMILES: CC(C)S(=O)(=O)c1ccc(OC(F)(F)F)c(Nc2ccc3c(n2)N(C2CCOCC2)[C@H](C)C(=O)N3C)c1] | (3R)-6-{[5- (Isopropylsulphonyl)-2- (trifluoromethoxy)phenyl] amino}-1,3-dimethyl-4- (tetrahydro-2H-pyran-4-yl)- 3,4-dihydropyrido[2,3- b]pyrazin-2(1H)-one | Intermediate 4, Intermediate 34 | ¹H-NMR: (400 MHz, 25° C., DMSO-d6): δ = 1.06 (d, 3H); 1.14 (dd, 6H); 1.50 (bd, 1H); 1.65 (qd, 1H); 1.73-1.87 (m, 2H); 3.22 (s, 3H); 3.37- 3.58 (m, 3H); 3.75- 3.86 (m, 2H); 4.23 (q, 1H); 4.45 (tt, 1H); 6.59 (d, 1H); 7.35 (d, 1H); 7.41 (dd, 1H); 7.57-7.62 (m, 1H); 8.62 (d, 1H); 8.77 (s, 1H). |
| 39 | 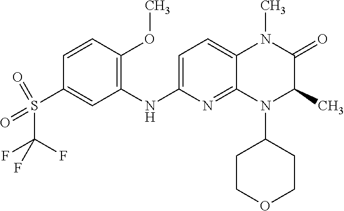 [SMILES: COc1ccc(S(=O)(=O)C(F)(F)F)cc1Nc1ccc2c(n1)N(C1CCOCC1)[C@H](C)C(=O)N2C] | (3R)-6-({2-Methoxy-5- [(trifluoromethyl)sulphonyl] phenyl}amino)-1,3- dimethyl-4-(tetrahydro-2H- pyran-4-yl)-3,4- dihydropyrido[2,3- b]pyrazin-2(1H)-one | Intermediate 4, 2-methoxy-5- [(trifluoro- methyl) sulphonyl] aniline (CAS 780-90-5) | ¹H-NMR: (400 MHz, 25° C., DMSO-d6): δ = 1.06 (d, 3H); 1.51 (bd, 1H); 1.68-1.88 (m, 3H); 3.22 (s, 3H); 3.45 (dt, 2H); 3.81-3.89 (m, 2H); 4.05 (s, 3H); 4.23 (q, 1H); 4.52 (tt, 1H); 6.67 (d, 1H); 7.16 (dt, 1H); 7.32- 7.40 (m, 3H); 7.47 (td, 1H); 7.61 (dd, 1H); 8.10 (d, 1H); 8.41 (s, 1H). |
| 40 | 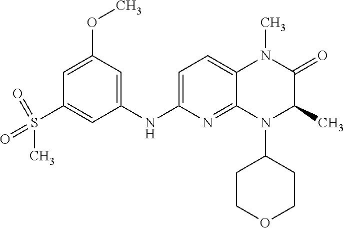 [SMILES: COc1cc(Nc2ccc3c(n2)N(C2CCOCC2)[C@H](C)C(=O)N3C)cc(S(C)(=O)=O)c1] | (3R)-6-{[3-Methoxy-5- (methylsulphonyl)phenyl] amino}-1,3-dimethyl-4- (tetrahydro-2H-pyran-4-yl)- 3,4-dihydropyrido[2,3- b]pyrazin-2(1H)-one | Intermediate 4, 2-methoxy-5- (methyl- sulphonyl) aniline (CAS 20945-70-4) | ¹H-NMR: (400 MHz, 25° C., DMSO-d6): δ = 1.08 (d, 3H); 1.56 (bd, 1H); 1.74 (qd, 1H); 1.85 (qd, 1H); 1.94 (bd, 1H); 3.20 (s, 3H); 3.21 (s, 3H); 3.47-3.59 (m, 2H); 3.83 (s, 3H); 3.88-3.96 (m, 2H); 4.24 (q, 1H); 4.52 (tt, 1H); 6.27 (d, 1H); 6.90 (t, 1H); 7.30 (d, 1H); 7.61 (t, 1H); 7.66 (t, 1H); 9.25 (s, 1H). |
| 41 | 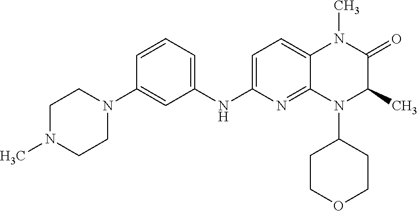 [SMILES: C[C@@H]1C(=O)N(C)c2ccc(Nc3cccc(N4CCN(C)CC4)c3)nc2N1C1CCOCC1] | (3R)-1,3-Dimethyl-6-{[3-(4- methylpiperazin-1- yl)phenyl]amino}-4- (tetrahydro-2H-pyran-4-yl)- 3,4-dihydropyrido[2,3- b]pyrazin-2(1H)-one | Intermediate 4, 3-(4-methyl- piperazin- 1-yl)aniline (CAS 148546-99-0) | ¹H-NMR: (400 MHz, 25° C., DMSO-d6): δ = 1.07 (d, 3H); 1.60 (bd, 1H); 1.75 (qd, 1H); 1.83-1.99 (m, 2H); 2.22 (s, 3H); 2.41-2.46 (m, 4H); 3.04-3.13 (m, 4H); 3.19 (s, 3H); 3.37- 3.48 (m, 2H); 3.96 (dt, 2H); 4.21 (q, 1H); 4.40 (tt, 1H); 6.22 (d, 1H); 6.46 (dd, 1H); 6.83 (t, 1H); 7.06 (t, 1H); 7.23 (d, 1H); 7.32 (dd, 1H); 8.59 (s, 1H). |
| 42 | 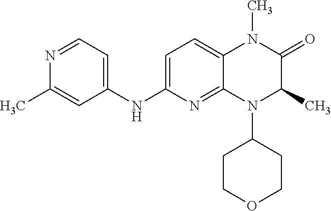 [SMILES: Cc1cc(Nc2ccc3c(n2)N(C2CCOCC2)[C@H](C)C(=O)N3C)ccn1] | (3R)-1,3-Dimethyl-6-[(2- methylpyridin-4-yl)amino]- 4-(tetrahydro-2H-pyran-4- yl)-3,4-dihydropyrido[2,3- b]pyrazin-2(1H)-one | Intermediate 4, 4-amino-2- methylpyridine (CAS 18437- 58-6) | ¹H-NMR: (400 MHz, 25° C., DMSO-d6): δ = 1.10 (d, 3H); 1.61 (bd, 1H); 1.82 (qd, 1H); 1.89-2.02 (m, 2H); 2.37 (s, 3H); 3.21 (s, 3H); 3.41- 3.50 (m, 2H); 3.95- 4.06 (m, 2H); 4.27 (q, 1H); 4.43 (tt, 1H); 6.32 (d, 1H); 7.31 (d, 1H); 7.34 (dd, 1H); 7.41 (d, 1H); 8.11 (d, 1H); 9.21 (s, 1H). |
| 43 | 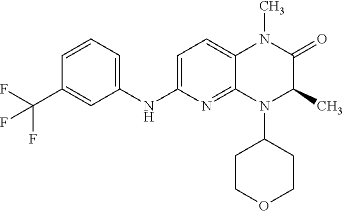 [SMILES: C[C@@H]1C(=O)N(C)c2ccc(Nc3cccc(C(F)(F)F)c3)nc2N1C1CCOCC1] | (3R)-1,3-Dimethyl-4- (tetrahydro-2H-pyran-4-yl)- 6-{[3-(trifluoromethyl) phenyl]amino}-3,4- dihydropyrido[2,3- b]pyrazin-2(1H)-one | Intermediate 4, 3-(trifluoro- methyl) aniline (CAS 98-16-8) | ¹H-NMR: (400 MHz, 25° C., DMSO-d6): δ = 1.08 (d, 3H); 1.57 (bd, 1H); 1.79 (qd, 1H); 1.84-1.96 (m, 2H); 3.21 (s, 3H); 3.35-3.45 (m, 2H); 3.90-4.01 (m, 2H); 4.24 (q, 1H); 4.45 (tt, 1H); 6.28 (d, 1H); 7.13 (d, 1H); 7.31 (d, 1H); 7.45 (t, 1H); 7.80 (d, 1H); 8.00 (s, 1H); 9.18 (s, 1H). |

### Example 44

### (3R)-1,3-Dimethyl-6-{[3-(S-methylsulphonimidoyl) phenyl]amino}-4-(tetrahydro-2H-pyran-4-yl)-3,4-dihydropyrido[2,3-b]pyrazin-2(1H)-one

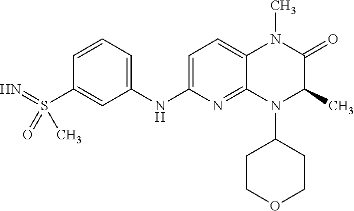 [SMILES: C[C@@H]1C(=O)N(C)c2ccc(Nc3cccc(S(C)(=N)=O)c3)nc2N1C1CCOCC1]

0.06 ml of sodium methoxide solution (30% in methanol) was added to a solution of 50 mg of ethyl [(3-{[(3R)-1,3-dimethyl-2-oxo-4-(tetrahydro-2H-pyran-4-yl)-1,2,3,4-tetrahydropyrido[2,3-b]pyrazin-6-yl]amino J}phenyl)(methyl)oxido-λ⁶-sulphanylidene]carbamate (example 10) in 3 ml of methanol and the mixture stirred at 60° C. for 4 hours. A further 0.06 ml of sodium methoxide solution (30% in methanol) was added and the mixture was stirred at 60° C. for a further 4 hours. The mixture was concentrated under reduced pressure, diluted with dichloromethane, applied to Isolute® and chromatographed on silica gel (dichloromethane/methanol gradient to 1% methanol content). This gave 33 mg of (3R)-1,3-dimethyl-6-{[3-(S-methylsulphonimidoyl)phenyl]amino}-4-(tetrahydro-2H-pyran-4-yl)-3,4-dihydropyrido[2,3-b]pyrazin-2(1H)-one.

¹H-NMR: (400 MHz, 25° C., DMSO-d6): δ=1.02-1.13 (m, 3H); 1.58 (bd, 1H); 1.70-2.00 (m, 3H); 3.03 (s, 3H); 3.21 (s, 3H); 3.48-3.60 (m, 2H); 3.87-4.00 (m, 2H); 4.09 (s, 1H); 4.23 (q, 1H); 4.44-4.55 (m, 1H); 6.29 (d, 1H); 7.29 (d, 1H); 7.35 (d, 1H); 7.44 (t, 1H); 7.95-8.06 (m, 2H); 9.19 (s, 1H).

### Biological Efficacy of the Compounds According to the Invention

### Protein-Protein Interaction Assay: BRD4/Acetylated Peptide H4 Binding Assay

### 1. Assay Description for BRD4 Bromo Domain 1 [BRD4(1)]

To assess the BRD4(1) binding strength of the substances described in this application, the ability thereof to inhibit the interaction between BRD4(1) and acetylated histone H4 in a dose-dependent manner was quantified.

For this purpose, a time-resolved fluorescence resonance energy transfer (TR-FRET) assay was used, which measures the binding between N-terminally His6-tagged BRD4(1) (amino acids 67-152) and a synthetic acetylated histone H4 (Ac-H4) peptide with sequence GRGK(Ac)GGK(Ac)GLGK(Ac)GGAK(Ac)RHGSGSK-biotin. The recombinant BRD4(1) protein produced in-house according to Filippakopoulos et al., Cell, 2012, 149:214-231 was expressed in E. coli and purified by means of (Ni-NTA) affinity and (Sephadex G-75) size exclusion chromatography. The Ac-H4 peptide can be purchased, for example, from Biosyntan (Berlin, Germany).

In the assay, typically 11 different concentrations of each substance (0.1 nM, 0.33 nM, 1.1 nM, 3.8 nM, 13 nM, 44 nM, 0.15 μM, 0.51 μM, 1.7 μM, 5.9 μM and 20 μM) were analysed as duplicates on the same microtitre plate. For this purpose, 100-fold concentrated solutions in DMSO were prepared by serial dilutions (1:3.4) of a 2 mM stock solution into a clear, 384-well microtitre plate (Greiner Bio-One, Frickenhausen, Germany). From this, 50 nl were transferred into a black test plate (Greiner Bio-One, Frickenhausen, Germany). The test was started by the addition of 2 μl of a 2.5-fold concentrated BRD4(1) solution (final concentration typically 10 nM in the 5 μl of reaction volume) in aqueous assay buffer [50 mM HEPES pH 7.5, 50 mM sodium chloride (NaCl), 0.25 mM CHAPS and 0.05% serum albumin (BSA)] to the substances in the test plate. This was followed by a 10-minute incubation step at 22° C. for the pre-equilibration of putative complexes between BRD4(1) and the substances. Subsequently, 3 μl of a solution of 1.67 times the concentration (in assay buffer) consisting of Ac-H4 peptide (83.5 nM) and TR-FRET detection reagents [16.7 nM anti-6His-XL665 and 3.34 nM streptavidin cryptate (both from Cisbio Bioassays, Codolet, France), and 668 mM potassium fluoride (KF)] were added.

The mixture was then incubated in the dark at 22° C. for one hour and then at 4° C. for at least 3 hours and for no longer than overnight. The formation of BRD4(1)/Ac-H4 complexes was determined by the measurement of the resonance energy transfer from the streptavidin-Eu cryptate to the anti-6His-XL665 antibody present in the reaction. For this purpose, the fluorescence emission was measured at 620 nm and 665 nm after excitation at 330-350 nm in a TR-FRET measuring instrument, for example a Rubystar or Pherastar (both from BMG Lab Technologies, Offenburg, Germany) or a Viewlux (Perkin-Elmer). The ratio of the emissions at 665 nm and at 622 nm was taken as an indicator of the amount of BRD4(1)/Ac-H4 complexes formed.

The data (ratios) obtained were normalized, with 0% inhibition corresponding to the mean from the measurements for a set of controls (typically 32 data points) in which all the reagents were present. In these, in place of test substances, 50 nl of DMSO (100%) were used. Inhibition of 100% corresponded to the mean from the measurements for a set of controls (typically 32 data points) in which all the reagents except BRD4(1) were present. The IC₅₀ was determined by regression analysis based on a 4-parameter equation (minimum, maximum, IC₅₀, Hill; Y=Max+(Min−Max)/(1+(X/IC₅₀)Hill).

### 2. Assay Description for BRD4 Bromo Domain 2 [BRD4(2)]

To assess the BRD4(2) binding strength of the substances described in this application, the ability thereof to inhibit the interaction between BRD4(2) and acetylated histone H4 in a dose-dependent manner was quantified.

For this purpose, a time-resolved fluorescence resonance energy transfer (TR-FRET) assay was used, which measures the binding between N-terminally His6-tagged BRD4(2) (amino acids 357-445) and a synthetic acetylated histone H4 (Ac-H4) peptide with sequence SGRGK(Ac)GGK(Ac)GLGK(Ac)GGAK(Ac)RHRKVLRDNGSGSK-biotin. The recombinant BRD4(2) protein produced in-house according to Filippakopoulos et al., Cell, 2012, 149:214-231 was expressed in E. coli and purified by means of (Ni-NTA) affinity and (Sephadex G-75) size exclusion chromatography. The Ac-H4 peptide can be purchased, for example, from Biosyntan (Berlin, Germany).

In the assay, typically 11 different concentrations of each substance (0.1 nM, 0.33 nM, 1.1 nM, 3.8 nM, 13 nM, 44 nM, 0.15 μM, 0.51 μM, 1.7 μM, 5.9 μM and 20 μM) were analysed as duplicates on the same microtitre plate. For this purpose, 100-fold concentrated solutions in DMSO were prepared by serial dilutions (1:3.4) of a 2 mM stock solution into a clear, 384-well microtitre plate (Greiner Bio-One, Frickenhausen, Germany). From this, 50 nl were transferred into a black test plate (Greiner Bio-One, Frickenhausen, Germany). The test was started by the addition of 2 μl of a 2.5-fold concentrated BRD4(2) solution (final concentration typically 100 nM in the 5 μl of reaction volume) in aqueous assay buffer [50 mM HEPES pH 7.5, 50 mM sodium chloride (NaCl); 50 mM potassium fluoride (KF); 0.25 mM CHAPS and 0.05% serum albumin (BSA)] to the substances in the test plate. This was followed by a 10-minute incubation step at 22° C. for the pre-equilibration of putative complexes between BRD4(2) and the substances. Subsequently, 3 μl of a 1.67-fold concentrated solution (in assay buffer) consisting of Ac-H4 peptide (83.5 nM) and TR-FRET detection reagents [83.5 nM anti-6His-XL665 (Cisbio Bioassays, Codolet, France) and 12.52 nM streptavidin-Eu), (Perkin Elmer, # W1024)] in assay buffer were added.

The mixture was then incubated in the dark at 22° C. for one hour and then at 4° C. for at least 3 hours and for no longer than overnight. The formation of BRD4(2)/Ac-H4 complexes was determined by the measurement of the resonance energy transfer from the streptavidin-Eu chelate to the anti-6His-XL665 antibody present in the reaction. For this purpose, the fluorescence emission was measured at 620 nm and 665 nm after excitation at 330-350 nm in a TR-FRET measuring instrument, for example a Rubystar or Pherastar (both from BMG Lab Technologies, Offenburg, Germany) or a Viewlux (Perkin-Elmer). The ratio of the emissions at 665 nm and at 622 nm was taken as an indicator of the amount of BRD4(2)/Ac-H4 complexes formed.

The data (ratios) obtained were normalized, with 0% inhibition corresponding to the mean from the measurements for a set of controls (typically 32 data points) in which all the reagents were present. In these, in place of test substances, 50 nl of DMSO (100%) were used. Inhibition of 100% corresponded to the mean from the measurements for a set of controls (typically 32 data points) in which all the reagents except BRD4(2) were present. The IC₅₀ was determined by regression analysis based on a 4-parameter equation (minimum, maximum, IC₅₀, Hill; Y=Max+(Min−Max)/(1+(X/IC₅₀)Hill)).

### 3. Cell Assay

### Cell Proliferation Assay

### Cell Proliferation Assay

In accordance with the invention, the ability of the substances to inhibit cell proliferation was determined. Cell viability was determined by means of the alamarBlue® reagent (Invitrogen) in a Victor X3 Multilabel Reader (Perkin Elmer). The excitation wavelength was 530 nm and the emission wavelength 590 nM.

The MOLM-13 cells (DSMZ, ACC 554) were seeded at a concentration of 4000 cells/well in 100 μl of growth medium (RPMI1640, 10% FCS) on 96-well microtitre plates (method 1).

Alternatively, the MOLM-13 cells (DSMZ, ACC 554) were seeded at a concentration of 500 cells/well in 5 μl of growth medium (RPMI1640, 10% FCS) on 1536-well microtitre plates (method 2).

The MOLP-8 cells (DSMZ, ACC 569) were seeded at a concentration of 4000 cells/well in 100 μl of growth medium (RPMI1640, 20% FCS) on 96-well microtitre plates.

The B16F10 cells (ATCC, CRL-6475) were seeded at a concentration of 300-500 cells/well in 100 μl of growth medium (DMEM with phenol red, 10% FCS) on 96-well microtitre plates.

The CHL-1 cells (ATCC, CRL-9446) were seeded at a concentration of 1000 cells/well in 100 μl of growth medium (DMEM with glutamine, 10% FCS) on 96-well microtitre plates.

After overnight incubation at 37° C., the fluorescence values (CI values) were determined. Then the plates were treated with various substance dilutions (1E-5 M, 3E-6 M, 1E-6 M, 3E-7 M, 1E-7 M, 3E-8 M, 1E-8 M) and incubated at 37° C. over 96 hours (MOLM-13, B16F10, CHL-1 cells) or 120 hours (MOLP-8 cells). Subsequently, the fluorescence values were determined (CO values). For the data analysis, the CI values were subtracted from the CO values and the results were compared between cells which had been treated with various dilutions of the substance or only with buffer solution. The IC50 values (substance concentration needed for 50% inhibition of cell proliferation) were calculated therefrom.

In method 2, after 3-day incubation with the inhibitor, the ATP concentration was determined as readout for the cell number using the CellTiterGlo kit (Promega). The measurement was carried out using a luminometer.

The substances were tested in the cell lines in Table 2, which represent the indications specified by way of example:

TABLE 2

| Cell line | Source | Indication |
|---|---|---|
| MOLM-13 | DSMZ | acute myeloid leukaemia |
| MOLP-8 | DSMZ | multiple myeloma |
| B16F10 | ATCC | melanoma (BRAF wild-type) |
| CHL-1 | ATCC | melanoma (BRAF wild-type) |

### 4. Results

### 4.1 Binding Assay

Table 3 shows the results from the BRD4(1) binding assay.

TABLE 3

| | IC₅₀ [BRD4(1)] |
|---|---|
| Example | (nmol/l) |
| 1 | 75 |
| 2 | 83 |
| 3 | 79 |
| 4 | 93 |
| 5 | 152 |
| 6 | 24 |
| 7 | 28 |
| 8 | 23 |
| 9 | 25 |
| 10 | 3630 |
| 11 | 94 |
| 12 | 19 |
| 13 | 16 |
| 14 | 173 |
| 15 | 73 |
| 16 | 88 |
| 17 | 303 |
| 18 | 1100 |
| 19 | 15 |
| 20 | 18 |
| 21 | 23 |
| 22 | 23 |
| 23 | 27 |
| 24 | 86 |
| 25 | 156 |
| 26 | 19 |
| 27 | 54 |
| 28 | 24 |
| 29 | 24 |
| 30 | 20 |
| 31 | 23 |
| 32 | 945 |
| 33 | 15 |
| 34 | 22 |
| 35 | 32 |
| 36 | 34 |
| 37 | 35 |
| 38 | 45 |
| 39 | 46 |
| 40 | 63 |
| 41 | 133 |
| 42 | 228 |
| 43 | 321 |
| 44 | 3640 |

Table 4 shows the results from the BRD4(2) binding assay.

TABLE 4

| | IC₅₀ [BRD4(2)] |
|---|---|
| Example | (nmol/l) |
| 1 | 121 |
| 2 | 173 |
| 3 | 58 |
| 4 | 64 |
| 5 | 96 |
| 6 | 40 |
| 7 | 44 |
| 8 | 49 |
| 9 | 53 |
| 10 | 6120 |
| 11 | 75 |
| 12 | 67 |
| 13 | 62 |
| 14 | 76 |
| 15 | 49 |
| 16 | 33 |
| 17 | 212 |
| 18 | 294 |
| 19 | 47 |
| 20 | 65 |
| 21 | 58 |
| 22 | 54 |
| 23 | 64 |
| 24 | 78 |
| 25 | 135 |
| 26 | 52 |
| 27 | 83 |
| 28 | 63 |
| 29 | 67 |
| 30 | 114 |
| 31 | 68 |
| 32 | 29 |
| 33 | 51 |
| 34 | 44 |
| 35 | 51 |
| 36 | 49 |
| 37 | 39 |
| 38 | 52 |
| 39 | 56 |
| 40 | 94 |
| 41 | 630 |
| 42 | 180 |
| 43 | 234 |
| 44 | 7340 |

### 4.2 Cell Proliferation Assay

Table 5 shows the results from the cell proliferation assays.

TABLE 5
The ability of the compounds according to the invention to
inhibit the proliferation of various cell lines was determined.

| | IC₅₀ | IC₅₀ | | | |
|---|---|---|---|---|---|
| | (MOLM-13) | (MOLM-13) | IC₅₀ | IC₅₀ | IC₅₀ |
| Exam- | (nmol/l) | (nmol/l) | (MOLP-8) | (B16F10) | (CHL-1) |
| ple | Method 1 | Method 2 | (nmol/l) | (nmol/l) | (nmol/l) |
| 1 | 191 | | 204 | 285 | 180 |
| 2 | 238 | | 236 | 319 | 189 |
| 3 | 175 | | 103 | 199 | 127 |
| 4 | 289 | | 221 | 375 | 196 |
| 5 | 651 | | 560 | 611 | 371 |
| 6 | 89 | | 67 | 87 | 46 |
| 7 | 111 | | 75 | 104 | 51 |
| 8 | 24 | | 23 | 37 | 18 |
| 9 | 42 | | 38 | 34 | 19 |
| 10 | 368 | | 281 | 304 | 196 |
| 11 | 188 | | 167 | 445 | 109 |
| 12 | 20 | | 22 | 81 | 13 |
| 13 | 15 | | 15 | 59 | 11 |
| 14 | 205 | | 174 | 248 | 118 |
| 15 | 149 | | 118 | 132 | 52 |
| 16 | 184 | | 135 | 180 | 76 |
| 17 | 1410 | | 1060 | 1100 | 800 |
| 18 | | 1650 | | | |
| 19 | 13 | | | 45 | 17 |
| 20 | 19 | | | 49 | 20 |
| 21 | 16 | | | 52 | 16 |
| 22 | 99 | | | 140 | 21 |
| 23 | | 51 | | | |
| 24 | | 147 | | | |
| 25 | | 576 | | | |
| 26 | 10 | | | 147 | 22 |
| 27 | 69 | | | | 311 |
| 28 | 26 | | | 21 | 25 |
| 29 | | | | 40 | |
| 30 | | 73 | | | |
| 31 | 14 | | | | 12 |
| 32 | | 40 | | | |
| 33 | | 26 | | | |
| 34 | 32 | | | 32 | 30 |
| 35 | 12 | | | 16 | 10 |
| 36 | | 160 | | | |
| 37 | | 124 | | | |
| 38 | 32 | | | 16 | 24 |
| 39 | 137 | | | 84 | |
| 40 | 121 | | | 269 | |
| 41 | | 708 | | | |
| 42 | 686 | | 436 | 1080 | 445 |
| 43 | 1220 | | 911 | 1080 | 707 |
| 44 | | >10000 | | | |

1: A compound of the formula (I) 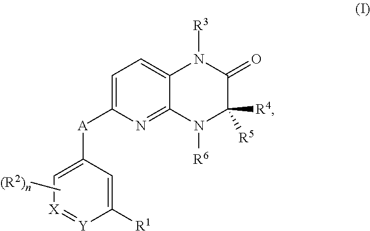
in which
A is —NH—, —N(C₁-C₃-alkyl)- or —O—,
X is —N—, —CH— or —CR²—,
Y is —N—, —CH— or —CR²—,
n is 0, 1 or 2,
R¹ is halogen, C₁-C₄-alkyl-, halo-C₁-C₄-alkyl-, cyano, —S(═O)₂R⁷, —S(═O)(═NR⁸)R⁹, —C(═O)R⁷ or —NR¹⁰R¹¹,
or
is phenyl-, which is unsubstituted or is mono-, di- or trisubstituted identically or differently by halogen, cyano, C₁-C₄-alkyl-, C₂-C₄-alkenyl-, C₂-C₄-alkynyl-, halo-C₁-C₄-alkyl-, C₁-C₄-alkoxy-,
halo-C₁-C₄-alkoxy-, C₁-C₄-alkylthio-, halo-C₁-C₄-alkylthio-,
—NR¹⁰R¹¹, —C(═O)OR¹², —C(═O)NR¹⁰R¹¹, —C(═O)R¹², —S(═O)₂R¹², —S(═O)₂NR¹⁰R¹¹,
or
is oxazolin-2-yl which is unsubstituted or is mono- or disubstituted identically or differently by C₁-C₃-alkyl,
R² is hydrogen, hydroxyl, halogen, cyano, nitro, C₁-C₃-alkyl-, C₂-C₄-alkenyl-, C₂-C₄-alkynyl-, halo-C₁-C₄-alkyl-, C₁-C₄-alkoxy-, halo-C₁-C₄-alkoxy-, C₁-C₄-alkylthio-, halo-C1-C₄-alkylthio-, phenyl- or phenoxy-,
where phenyl- and the phenyl-present in phenoxy- are unsubstituted or are mono-, di- or trisubstituted identically or differently by halogen, cyano, C₁-C₃-alkyl- or C1-C₃-alkoxy-, and
if n is 2, R² may be identical or different,
or
R¹ and R² together are a group *—S(═O)₂—CH₂—CH₂—** or *—S(═O)₂—CH₂—CH₂—CH₂—**, where “*” signifies the point of attachment of R¹ to the phenyl ring or 6-membered heteroaryl ring to which R¹ is bonded, and where “**” signifies a carbon atom of this ring adjacent to this point of attachment,
R³ is methyl- or ethyl-,
R⁴ is hydrogen or C₁-C₃-alkyl-,
R⁵ is hydrogen or C₁-C₃-alkyl-,
or
R⁴ and R⁵ together are C₂-C₅-alkylene,
R⁶ is C₁-C₆-alkyl- which is unsubstituted or monosubstituted by C₁-C₃-alkoxy-, phenyl-, C₃-C₈-cycloalkyl-, or 4- to 8-membered heterocycloalkyl-,
where phenyl- for its part is unsubstituted or is mono-, di- or trisubstituted identically or differently by halogen, cyano, C₁-C₄-alkyl-, C₂-C₄-alkenyl-, C₂-C₄-alkynyl-, C₁-C₄-alkoxy-, halo-C₁-C₄-alkyl- or halo-C₁-C₄-alkoxy-, and
where C₃-C₈-cycloalkyl- and 4- to 8-membered heterocycloalkyl- for their part are unsubstituted or are mono- or disubstituted identically or differently by C₁-C₃-alkyl-,
or
is C₃-C₈-cycloalkyl- or 4- to 8-membered heterocycloalkyl-, which are unsubstituted or are mono- or disubstituted identically or differently by C₁-C₃-alkyl-, halo-C₁-C₃-alkyl- or C₁-C₄-alkoxycarbonyl-,
or
is phenyl or 5- to 6-membered heteroaryl- which are unsubstituted or are mono- or disubstituted identically or differently by halogen, C₁-C₃-alkyl- or 4- to 8-membered heterocycloalkyl-,
where the 4- to 8-membered heterocycloalkyl- for its part is unsubstituted or mono- or disubstituted identically or differently by C₁-C₃-alkyl- or C₁-C₄-alkoxycarbonyl-,
R⁷ is C₁-C₆-alkyl- which is unsubstituted or is monosubstituted by cyano, C₁-C₃-alkoxy-, C₁-C₃-alkylamino-, phenyl-, C₃-C₈-cycloalkyl- or 4- to 8-membered heterocycloalkyl-,
where phenyl- for its part is unsubstituted or is mono-, di- or trisubstituted identically or differently by halogen, cyano, C₁-C₄-alkyl-, C₂-C₄-alkenyl-, C₂-C₄-alkynyl-, C₁-C₄-alkoxy-, halo-C₁-C₄-alkyl- or halo-C₁-C₄-alkoxy-, and
where C₃-C₈-cycloalkyl- and 4- to 8-membered heterocycloalkyl- for their part are unsubstituted or are mono- or disubstituted identically or differently by C₁-C₃-alkyl-,
or
is halo-C₁-C₄-alkyl-,
or
is C₂-C₄-alkenyl- or C₂-C₄-alkynyl-,
or
is C₃-C₈-cycloalkyl- or 4- to 8-membered heterocycloalkyl- which are unsubstituted or are mono- or disubstituted identically or differently by C₁-C₃-alkyl- or C₁-C₄-alkoxycarbonyl-, with the proviso that the 4- to 8-membered heterocycloalkyl- is not bonded via a nitrogen atom to the carbonyl or sulphonyl group in R¹,
R⁸ is hydrogen, cyano, C₁-C₆-alkyl-, C₃-C₈-cycloalkyl- or —C(═O)OR¹²,
R⁹ is C₁-C₆-alkyl or C₃-C₈-cycloalkyl,
R¹⁰ and R¹¹ are each independently hydrogen or are unsubstituted C₁-C₃-alkyl- or C₁-C₃-alkyl-mono- or disubstituted identically or differently by hydroxyl, oxo, C₁-C₃-alkoxy-, or are fluoro-C₁-C₃-alkyl- or 4- to 8-membered heterocycloalkyl-,
where the 4- to 8-membered heterocycloalkyl- for its part is unsubstituted or mono- or disubstituted identically or differently by C₁-C₃-alkyl-,
or
R¹⁰ and R¹¹ together with the nitrogen atom to which they are bonded are
4- to 8-membered heterocycloalkyl-, which is unsubstituted or is mono- or disubstitued identically or differently by hydroxyl, fluorine, oxo, cyano, C₁-C₃-alkyl-, fluoro-C₁-C₃-alkyl-, C₃-C₆-cycloalkyl-, cyclopropylmethyl-, C₁-C₃-alkylcarbonyl- or C₁-C₄-alkoxycarbonyl-, and
R¹² is C₁-C₆-alkyl- or phenyl-C₁-C₃-alkyl-,
and diastereomers, racemates, polymorphs and physiologically acceptable salts thereof.

2: The compound of the formula (I) according to claim 1, in which
A is —NH— or —N(methyl)-,
X is —N— or —CH—,
Y is —N— or —CH—,
n is 0, 1 or 2,
R¹ is C₁-C₃-alkyl-, fluoro-C₁-C₃-alkyl-, —S(═O)₂R⁷, —S(═O)(═NR⁸)R⁹ or
—NR¹⁰R¹¹,
or
is phenyl- which is unsubstituted or is mono-, di- or trisubstituted identically or differently by halogen, cyano, C₁-C₃-alkyl-, trifluoromethyl-, C₁-C₃-alkoxy-, trifluoromethoxy- or —NR¹⁰R¹¹,
or
is oxazolin-2-yl- which is unsubstituted or is mono- or disubstituted identically or differently by C₁-C₃-alkyl-,
R² is hydrogen, hydroxyl, fluorine, chlorine, cyano, methyl-, ethyl-, methoxy-, ethoxy-, trifluoromethoxy- or phenoxy-, where the phenyl-present in phenoxy- is unsubstituted or is mono- or disubstituted identically or differently by fluorine, chlorine, bromine, cyano, methyl- or methoxy-, and, if n is 2, R² may be identical or different,
or
R¹ and R² together are a group *—S(═O)₂—CH₂—CH₂—** or *—S(═O)₂—CH₂—CH₂—CH₂—**, where “*” signifies the point of attachment of R¹ to the phenyl ring or 6-membered heteroaryl ring to which R¹ is bonded, and where “**” signifies a carbon atom of this ring adjacent to this point of attachment,
R³ is methyl- or ethyl-,
R⁴ is hydrogen, methyl- or ethyl-,
R⁵ is hydrogen, methyl- or ethyl-,
R⁶ is C₂-C₅-alkyl- which is unsubstituted,
or
is methyl- or ethyl-monosubstituted by
C₁-C₃-alkoxy-, phenyl- or 4- to 8-membered heterocycloalkyl-,
where phenyl- for its part is unsubstituted or is mono-, di- or trisubstituted identically or differently by fluorine, chlorine, bromine, cyano, C₁-C₃-alkyl- or C₁-C₃-alkoxy-, and
where the 4- to 8-membered heterocycloalkyl- for its part is unsubstituted or is mono- or disubstituted by methyl-,
or
is C₃-C₈-cycloalkyl- or 4- to 8-membered heterocycloalkyl-, which are unsubstituted or are mono- or disubstituted identically or differently by C₁-C₃-alkyl-, fluoro-C₁-C₃-alkyl- or C₁-C₄-alkoxycarbonyl-,
or
is phenyl or 5- to 6-membered heteroaryl- which are unsubstituted or are mono- or disubstituted identically or differently by fluorine, chlorine, methyl- or 6-membered heterocycloalkyl-,
in which the 6-membered heterocycloalkyl- for its part is unsubstituted or is monosubstituted by methyl- or tert-butoxycarbonyl-,
R⁷ is C₁-C₆-alkyl- which is unsubstituted or is monosubstituted by cyano, C₁-C₃-alkoxy-, C₁-C₃-alkylamino-, phenyl- or 4- to 8-membered heterocycloalkyl-,
where phenyl- for its part is unsubstituted or is mono-, di- or trisubstituted identically or differently by fluorine, chlorine, bromine, cyano, C₁-C₃-alkyl-, C₁-C₃-alkoxy-, and
where the 4- to 8-membered heterocycloalkyl- for its part is unsubstituted or mono- or disubstituted identically or differently by C₁-C₃-alkyl-,
or
is fluoro-C₁-C₃-alkyl-,
or
is C₃-C₄-alkenyl- or C₃-C₄-alkynyl-,
or
is C₃-C₈-cycloalkyl- or 4- to 8-membered heterocycloalkyl- which are unsubstituted or are mono- or disubstituted identically or differently by C₁-C₃-alkyl- or C₁-C₄-alkoxycarbonyl-, with the proviso that the 4- to 8-membered heterocycloalkyl- is not bonded via a nitrogen atom to the sulphonyl group in R¹,
R⁸ is hydrogen, cyano, C₁-C₄-alkyl-, C₃-C₆-cycloalkyl- or —C(═O)OR¹²,
R⁹ is (C₁-C₄)-alkyl-,
R¹⁰ and R¹¹ are each independently hydrogen or are unsubstituted C₁-C₃-alkyl- or C₁-C₃-alkyl-monosubstituted by hydroxyl or oxo or are 5- to 6-membered heterocycloalkyl-,
where the 5- to 6-membered heterocycloalkyl- for its part is unsubstituted or mono- or disubstituted identically or differently by C₁-C₃-alkyl-,
or
R¹⁰ and R¹¹ together with the nitrogen atom to which they are bonded are
4- to 7-membered heterocycloalkyl-, which is unsubstituted or is mono- or disubstitued identically or differently by hydroxyl, fluorine, oxo, C₁-C₃-alkyl-, fluoro-C₁-C₃-alkyl-, cyclopropyl-, cyclopropylmethyl-, acetyl- or tert-butoxycarbonyl-, and
R¹² is C₁-C₄-alkyl- or benzyl-,
and diastereomers, racemates, polymorphs and physiologically acceptable salts thereof.

3: The compound of the formula (I) according to claim 1 in which
A is —NH—,
X is —CH—,
Y is —N— or —CH—,
n is 0 or 1,
R¹ is C₁-C₂-alkyl-, fluoro-C₁-C₂-alkyl-, —S(═O)₂R⁷, —S(═O)(═NR⁸)R⁹ or —NR¹⁰R¹¹,
or
is phenyl-, which is unsubstituted or is mono- or disubstituted identically or differently by fluorine, chlorine, bromine, cyano, methyl-, trifluoromethyl- or methoxy-,
or
is oxazolin-2-yl- which is unsubstituted or is mono- or disubstituted by methyl-,
R² is hydrogen, fluorine, chlorine, methyl-, methoxy-, trifluoromethoxy- or phenoxy-, where the phenyl-present in phenoxy- is unsubstituted or is monosubstituted by fluorine or chlorine,
or
R¹ and R² together are a group *—S(═O)₂—CH₂—CH₂—**, where “*” signifies the point of attachment of R¹ to the phenyl ring or pyridine ring to which R¹ is bonded, and where “**” signifies a carbon atom of this ring adjacent to this point of attachment,
R³ is methyl-,
R⁴ is methyl- or ethyl-,
R⁵ is hydrogen,
R⁶ is (C₃-C₅)-alkyl-,
or
is methyl-monosubstituted by phenyl- or 4- to 6-membered heterocycloalkyl-,
where phenyl- for its part is unsubstituted or is mono- or disubstituted identically or differently by fluorine, chlorine, cyano, methyl- or methoxy-, and
where the 4- to 6-membered heterocycloalkyl- for its part is unsubstituted or is monosubstituted by methyl-,
or
is C₃-C₈-cycloalkyl- or 4- to 6-membered heterocycloalkyl-, which are unsubstituted or are mono- or disubstituted identically or differently by C₁-C₃-alkyl-, fluoro-C₁-C₃-alkyl- or C₁-C₄-alkoxycarbonyl-,
or
is phenyl-, which is unsubstituted or is mono- or disubstituted identically or differently by fluorine, chlorine or methyl-,
R⁷ is C₁-C₄-alkyl- which is unsubstituted or is monosubstituted by cyano, phenyl- or 5- to 6-membered heterocycloalkyl-,
where phenyl- for its part is unsubstituted or is mono- or disubstituted identically or differently by fluorine, chlorine, cyano, methyl-, methoxy-, and
where the 5- to 6-membered heterocycloalkyl- for its part is unsubstituted or is monosubstituted by C₁-C₃-alkyl-,
or
is fluoro-C₁-C₂-alkyl-,
or
is C₃-C₄-alkenyl-,
or
is C₃-C₆-cycloalkyl- or 5- to 6-membered heterocycloalkyl-, with the proviso that the 5- to 6-membered heterocycloalkyl- is not bonded via a nitrogen atom to the sulphonyl group in R¹,
R⁸ is hydrogen, cyano, C₁-C₃-alkyl- or C₁-C₃-alkoxycarbonyl-,
R⁹ is C₁-C₃-alkyl-, and
R¹⁰ and R¹¹ are each independently hydrogen or C₁-C₃-alkyl-,
or
R¹⁰ and R¹¹ together with the nitrogen atom to which they are bonded are
4- to 7-membered heterocycloalkyl-, which is unsubstituted or is monosubstituted by oxo, C₁-C₃-alkyl-, cyclopropyl-, cyclopropylmethyl-, acetyl- or tert-butoxycarbonyl-,
and diastereomers, racemates, polymorphs and physiologically acceptable salts thereof.

4: The compound of the formula (I) according to claim 1 in which
A is —NH—,
X is —CH—,
Y is —N— or —CH—,
n is 0 or 1,
R¹ is methyl-, trifluoromethyl-, —S(═O)₂—R⁷ or —NR¹⁰R¹¹,
or
is phenyl-, which is unsubstituted or is monosubstituted by fluorine, chlorine, cyano, methyl-, methoxy-,
R² is hydrogen, methyl-, methoxy-, trifluoromethoxy-, phenoxy- or para-fluorophenoxy-,
or
R¹ and R² together are a group *—S(═O)₂—CH₂—CH₂—**, where “*” signifies the point of attachment of R¹ to the phenyl ring or pyridine ring to which R¹ is bonded, and where “**” signifies a carbon atom of this ring adjacent to this point of attachment,
R³ is methyl-,
R⁴ is methyl-,
R⁵ is hydrogen,
R⁶ is isopropyl-,
or
is cycloheptyl-,
or
is tetrahydropyranyl- or piperidinyl-, which are unsubstituted or are monosubstituted by methyl-, 2,2-difluoroethyl-, 2,2,2-trifluoroethyl-, 3,3,3-trifluoropropyl- or tert-butoxycarbonyl-,
or
is phenyl-, which is unsubstituted or is mono- or disubstituted identically or differently by fluorine, chlorine or methyl-,
R⁷ is C₁-C₃-alkyl-, trifluoromethyl-, allyl-, C₃-C₄-cycloalkyl- or is tetrahydropyranyl-, and
R¹⁰ and R¹¹ together with the nitrogen atom to which they are bonded are 5- to 6-membered heterocycloalkyl- which is unsubstituted or is monosubstituted by C₁-C₃-alkyl-,
and diastereomers, racemates, polymorphs and physiologically acceptable salts thereof.

5: The compound of the formula (I) according to claim 1 in which
A is —NH—,
X is —CH—,
Y is —N— or —CH—,
n is 0 or 1,
R¹ is methyl-, trifluoromethyl-, —S(═O)₂—R⁷, —NR¹⁰R¹¹ or is para-cyanophenyl-,
R² is hydrogen, methyl-, methoxy-, trifluoromethoxy-, phenoxy- or para-fluorophenoxy-,
or
R¹ and R² together with the phenyl ring to which they are bonded are 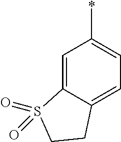 [SMILES: *c1ccc2c(c1)S(=O)(=O)CC2]
in which “*” signifies the point of attachment to the rest of the molecule,
R³ is methyl-,
R⁴ is methyl-,
R⁵ is hydrogen,
R⁶ is isopropyl-,
or
is cycloheptyl-,
or is tetrahydropyran-4-yl- or piperidin-4-yl-, where piperidin-4-yl- is unsubstituted or is monosubstituted on the nitrogen by methyl-, 2,2-difluoroethyl-, 2,2,2-trifluoroethyl-, 3,3,3-trifluoropropyl- or tert-butoxycarbonyl-,
or
is phenyl-,
R⁷ is methyl-, ethyl-, isopropyl-, trifluoromethyl-, allyl-, cyclopropyl-, cyclobutyl- or is tetrahydropyran-4-yl-, and
R¹⁰ and R¹¹ together with the nitrogen atom to which they are bonded are N-methylpiperazinyl-,
and diastereomers, racemates, polymorphs and physiologically acceptable salts thereof.

6: The compound of the formula (I) according to claim 1, selected from the group consisting of:
(3R)-1,3-Dimethyl-6-{[3-(methylsulphonyl)phenyl]amino}-4-(tetrahydro-2H-pyran-4-yl)-3,4-dihydropyrido[2,3-b]pyrazin-2(1H)-one;
(3R)-1,3-Dimethyl-6-{[2-methyl-5-(methylsulphonyl)phenyl]amino}-4-(tetrahydro-2H-pyran-4-yl)-3,4-dihydropyrido[2,3-b]pyrazin-2(1H)-one;
(3R)-6-{[2-Methoxy-5-(methylsulphonyl)phenyl]amino}-1,3-dimethyl-4-(tetrahydro-2H-pyran-4-yl)-3,4-dihydropyrido[2,3-b]pyrazin-2(1H)-one;
(3R)-6-[(1,1-Dioxido-2,3-dihydro-1-benzothiophen-6-yl)amino]-1,3-dimethyl-4-(tetrahydro-2H-pyran-4-yl)-3,4-dihydropyrido[2,3-b]pyrazin-2(1H)-one;
(3R)-4-Cycloheptyl-1,3-dimethyl-6-{[3-(methylsulphonyl)phenyl]amino}-3,4-dihydropyrido[2,3-b]pyrazin-2(1H)-one;
(3R)-6-{[3-(Cyclopropylsulphonyl)phenyl]amino}-1,3-dimethyl-4-(tetrahydro-2H-pyran-4-yl)-3,4-dihydropyrido[2,3-b]pyrazin-2(1H)-one;
(3R)-6-{[3-(Isopropylsulphonyl)phenyl]amino}-1,3-dimethyl-4-(tetrahydro-2H-pyran-4-yl)-3,4-dihydropyrido[2,3-b]pyrazin-2(1H)-one;
(3R)-6-{[5-(Cyclopropylsulphonyl)-2-methoxyphenyl]amino}-1,3-dimethyl-4-(tetrahydro-2H-pyran-4-yl)-3,4-dihydropyrido[2,3-b]pyrazin-2(1H)-one;
(3R)-6-{[5-(Isopropylsulphonyl)-2-methoxyphenyl]amino}-1,3-dimethyl-4-(tetrahydro-2H-pyran-4-yl)-3,4-dihydropyrido[2,3-b]pyrazin-2(1H)-one;
Ethyl [(3-{[(3R)-1,3-dimethyl-2-oxo-4-(tetrahydro-2H-pyran-4-yl)-1,2,3,4-tetrahydropyrido[2,3-b]pyrazin-6-yl]amino}phenyl)(methyl)oxido-λ⁶-sulphanylidene]carbamate;
(3R)-1,3-Dimethyl-4-(1-methylpiperidin-4-yl)-6-{[3-(methylsulphonyl)phenyl]amino}-3,4-dihydropyrido[2,3-b]pyrazin-2(1H)-one;
(3R)-6-{[5-(Cyclopropylsulphonyl)-2-methoxyphenyl]amino}-1,3-dimethyl-4-(1-methylpiperidin-4-yl)-3,4-dihydropyrido[2,3-b]pyrazin-2(1H)-one;
(3R)-6-{[5-(Isopropylsulphonyl)-2-methoxyphenyl]amino}-1,3-dimethyl-4-(1-methylpiperidin-4-yl)-3,4-dihydropyrido[2,3-b]pyrazin-2(1H)-one;
(3R)-4-Isopropyl-6-{[2-methoxy-5-(methylsulphonyl)phenyl]amino}-1,3-dimethyl-3,4-dihydropyrido[2,3-b]pyrazin-2(1H)-one;
(3R)-6-{[5-(Cyclopropylsulphonyl)-2-methoxyphenyl]amino}-4-isopropyl-1,3-dimethyl-3,4-dihydropyrido[2,3-b]pyrazin-2(1H)-one;
(3R)-4-Isopropyl-6-{[5-(isopropylsulphonyl)-2-methoxyphenyl]amino}-1,3-dimethyl-3,4-dihydropyrido[2,3-b]pyrazin-2(1H)-one;
1,3-Dimethyl-6-{[3-(methylsulphonyl)phenyl]amino}-4-phenyl-3,4-dihydropyrido[2,3-b]pyrazin-2(1H)-one;
3′-{[(3R)-1,3-Dimethyl-2-oxo-4-(tetrahydro-2H-pyran-4-yl)-1,2,3,4-tetrahydropyrido[2,3-b]pyrazin-6-yl]amino}biphenyl-4-carbonitrile;
(3R)-6-{[3-(Isopropylsulphonyl)phenyl]amino}-1,3-dimethyl-4-(1-methylpiperidin-4-yl)-3,4-dihydropyrido[2,3-b]pyrazin-2(1H)-one;
(3R)-6-{[5-(Isopropylsulphonyl)-2-(trifluoromethoxy)phenyl]amino}-1,3-dimethyl-4-(1-methylpiperidin-4-yl)-3,4-dihydropyrido[2,3-b]pyrazin-2(1H)-one;
(3R)-6-({2-Methoxy-5-[(trifluoromethyl)sulphonyl]phenyl}amino)-1,3-dimethyl-4-(1-methylpiperidin-4-yl)-3,4-dihydropyrido[2,3-b]pyrazin-2(1H)-one;
(3R)-6-{[2-Methoxy-5-(tetrahydro-2H-pyran-4-ylsulphonyl)phenyl]amino}-1,3-dimethyl-4-(1-methylpiperidin-4-yl)-3,4-dihydropyrido[2,3-b]pyrazin-2(1H)-one;
(3R)-6-{[5-(Allylsulphonyl)-2-methoxyphenyl]amino}-1,3-dimethyl-4-(1-methylpiperidin-4-yl)-3,4-dihydropyrido[2,3-b]pyrazin-2(1H)-one;
tert-Butyl 4-[(3R)-6-{[5-(isopropylsulphonyl)-2-methoxyphenyl]amino}-1,3-dimethyl-2-oxo-2,3-dihydropyrido[2,3-b]pyrazin-4(1H)-yl]piperidine-1-carboxylate;
tert-Butyl 4-[(3R)-6-{[2-methoxy-5-(methylsulphonyl)phenyl]amino}-1,3-dimethyl-2-oxo-2,3-dihydropyrido[2,3-b]pyrazin-4(1H)-yl]piperidine-1-carboxylate;
(3R)-6-{[5-(Isopropylsulphonyl)-2-methoxyphenyl]amino}-1,3-dimethyl-4-(piperidin-4-yl)-3,4-dihydropyrido[2,3-b]pyrazin-2(1H)-one;
(3R)-6-{[2-Methoxy-5-(methylsulphonyl)phenyl]amino}-1,3-dimethyl-4-(piperidin-4-yl)-3,4-dihydropyrido[2,3-b]pyrazin-2(1H)-one;
(3R)-6-{[5-(Isopropylsulphonyl)-2-methoxyphenyl]amino}-1,3-dimethyl-4-[1-(2,2,2-trifluoroethyl)piperidin-4-yl]-3,4-dihydropyrido[2,3-b]pyrazin-2(1H)-one;
(3R)-4-[1-(2,2-Difluoroethyl)piperidin-4-yl]-6-{[5-(isopropylsulphonyl)-2-methoxyphenyl]amino}-1,3-dimethyl-3,4-dihydropyrido[2,3-b]pyrazin-2(1H)-one;
(3R)-6-{[3-(Isopropylsulphonyl)phenyl]amino}-1,3-dimethyl-4-[1-(3,3,3-trifluoropropyl)piperidin-4-yl]-3,4-dihydropyrido[2,3-b]pyrazin-2(1H)-one;
(3R)-6-{[5-(Isopropylsulphonyl)-2-methoxyphenyl]amino}-1,3-dimethyl-4-[1-(3,3,3-trifluoropropyl)piperidin-4-yl]-3,4-dihydropyrido[2,3-b]pyrazin-2(1H)-one;
(3R)-6-{[5-(Ethylsulphonyl)-2-(4-fluorophenoxy)phenyl]amino}-1,3-dimethyl-4-(1-methylpiperidin-4-yl)-3,4-dihydropyrido[2,3-b]pyrazin-2(1H)-one;
(3R)-6-{[5-(Ethylsulphonyl)-2-phenoxyphenyl]amino}-1,3-dimethyl-4-(1-methylpiperidin-4-yl)-3,4-dihydropyrido[2,3-b]pyrazin-2(1H)-one;
(3R)-6-{[2-Methoxy-5-(tetrahydro-2H-pyran-4-ylsulphonyl)phenyl]amino}-1,3-dimethyl-4-(tetrahydro-2H-pyran-4-yl)-3,4-dihydropyrido[2,3-b]pyrazin-2(1H)-one;
(3R)-6-{[5-(Cyclobutylsulphonyl)-2-methoxyphenyl]amino}-1,3-dimethyl-4-(tetrahydro-2H-pyran-4-yl)-3,4-dihydropyrido[2,3-b]pyrazin-2(1H)-one;
(3R)-6-{[5-(Ethylsulphonyl)-2-phenoxyphenyl]amino}-1,3-dimethyl-4-(tetrahydro-2H-pyran-4-yl)-3,4-dihydropyrido[2,3-b]pyrazin-2(1H)-one;
(3R)-6-{[5-(Ethylsulphonyl)-2-(4-fluorophenoxy)phenyl]amino}-1,3-dimethyl-4-(tetrahydro-2H-pyran-4-yl)-3,4-dihydropyrido[2,3-b]pyrazin-2(1H)-one;
(3R)-6-{[5-(Isopropylsulphonyl)-2-(trifluoromethoxy)phenyl]amino}-1,3-dimethyl-4-(tetrahydro-2H-pyran-4-yl)-3,4-dihydropyrido[2,3-b]pyrazin-2(1H)-one;
(3R)-6-({2-Methoxy-5-[(trifluoromethyl)sulphonyl]phenyl}amino)-1,3-dimethyl-4-(tetrahydro-2H-pyran-4-yl)-3,4-dihydropyrido[2,3-b]pyrazin-2(1H)-one;
(3R)-6-{[3-Methoxy-5-(methylsulphonyl)phenyl]amino}-1,3-dimethyl-4-(tetrahydro-2H-pyran-4-yl)-3,4-dihydropyrido[2,3-b]pyrazin-2(1H)-one;
(3R)-1,3-Dimethyl-6-{[3-(4-methylpiperazin-1-yl)phenyl]amino}-4-(tetrahydro-2H-pyran-4-yl)-3,4-dihydropyrido[2,3-b]pyrazin-2(1H)-one;
(3R)-1,3-Dimethyl-6-[(2-methylpyridin-4-yl)amino]-4-(tetrahydro-2H-pyran-4-yl)-3,4-dihydropyrido[2,3-b]pyrazin-2(1H)-one;
(3R)-1,3-Dimethyl-4-(tetrahydro-2H-pyran-4-yl)-6-{[3-(trifluoromethyl)phenyl]amino}-3,4-dihydropyrido[2,3-b]pyrazin-2(1H)-one; and
(3R)-1,3-Dimethyl-6-{[3-(S-methylsulphonimidoyl) phenyl]amino}-4-(tetrahydro-2H-pyran-4-yl)-3,4-dihydropyrido[2,3-b]pyrazin-2(1H)-one,
and diastereomers, racemates, polymorphs and physiologically acceptable salts thereof.

7-9. (canceled)

10: A method for prophylaxis and/or therapy of neoplastic disorders, comprising administering to a patient a compound according to claim 1.

11: A method for prophylaxis and/or therapy of hyperproliferative disorders, comprising administering to a patient a compound according to claim 1.

12: A method for prophylaxis and/or therapy of viral infections, neurodegenerative disorders, inflammation disorders, or atherosclerotic disorders, or for male fertility control, comprising administering to a patient a compound according to claim 1.

13. (canceled)

14: A composition comprising a compound according to claim 1, in combination with one or more further pharmacologically active substances.

15: A method for prophylaxis and/or therapy of hyperproliferative disorders, comprising administering to a patient a composition according to claim 14.

16: Compounds in combination according to claim 14 A method for prophylaxis and/or therapy of neoplastic disorders, comprising administering to a patient a composition according to claim 14.

17: A method for prophylaxis and/or therapy of viral infections, neurodegenerative disorders, inflammatory disorders, or atherosclerotic disorders, or for male fertility control, comprising administering to a patient a composition according to claim 14.


SEQUENCE LISTING (2 sequences)

SEQ ID NO: 1 - protein, 22 aa, Artificial Sequence
  Acetylated histone H4 (Ac-H4) peptide, acetylated lysine at positions 4, 7, 11 and 15
GRGKGGKGLGKGGAKRHGSGSK

SEQ ID NO: 2 - protein, 30 aa, Artificial Sequence
  Acetylated histone H4 (Ac-H4) peptide, acetylated lysine at positions 5, 8, 12 and 16
SGRGKGGKGLGKGGAKRHRKVLRDNGSGSK